(19) United States
(12) Patent Application Publication
(10) Pub. No.: US 2010/0298302 A1
(43) Pub. Date: Nov. 25, 2010

(54) NOVEL PROTEIN KINASE MODULATORS

(76) Inventors: Fabrice PIERRE, La Jolla, CA (US); Mustapha Haddach, San Diego, CA (US); Peter C. Chua, Maple Ridge (CA)

Correspondence Address: COOLEY LLP;ATTN: Patent Group, Suite 1100, 777 - 6th Street, NW, WASHINGTON, DC, 20001, US

(21) Appl. No.: 12/784,055

(22) Filed: May 20, 2010

Related U.S. Application Data
(60) Provisional application No. 61/180,099, filed on May 20, 2009.
(60) Provisional application No. 61/218,347, filed on Jun. 18, 2009.
(60) Provisional application No. 61/287,646, filed on Dec. 17, 2009.

(51) Int. Cl. A61K 31/551; C07D 487/04; A61K 31/4985; C07D 243/08; C07D 413/14; A61K 31/5377; A61P 9/00; A61P 29/00; A61P 31/00; A61P 35/00; A61P 37/00

(52) U.S. Cl. 514/218; 544/344; 514/249; 540/575; 544/115; 514/233.2

(57) ABSTRACT

The invention provides compounds that inhibit selected kinases (Pim, Flt and/or CK2 kinases) and compositions containing such compounds. These compounds and compositions are useful for treating proliferative disorders such as cancer, as well as other kinase-associated conditions including inflammation, pain, and certain infections and immunological disorders.

### RELATED APPLICATIONS

This application claims benefit of priority to U.S. Provisional Application Ser. No. 61/180,099 filed 20 May 2009; U.S. Provisional Application Ser. No. 61/218,347 filed 18 Jun. 2009; and U.S. Provisional Application Ser. No. 61/287,646 filed 17 Dec. 2009. The contents of these documents are incorporated herein by reference in their entirety.

### FIELD OF THE INVENTION

The invention relates in part to molecules having certain biological activities that include, but are not limited to, inhibiting cell proliferation, modulating serine-threonine protein kinase activity and modulating tyrosine kinase activity. Molecules of the invention can modulate casein kinase (CK) activity (e.g., CK2 activity) and/or Pim kinase activity (e.g., PIM-1 activity), and/or Fms-like tyrosine kinase (Flt) activity (e.g., Flt-3 activity). These compounds are useful in treatment of various physiological disorders, due to their activity as kinase inhibitors. The invention also relates in part to methods for using such molecules, and compositions containing them.

### BACKGROUND

The PIM protein kinases, which include the closely related PIM-1, -2, and -3, have been implicated in diverse biological processes such as cell survival, proliferation, and differentiation. PIM-1 is involved in a number of signaling pathways that are highly relevant to tumorigenesis [reviewed in Bachmann & Moroy, Internat. J. Biochem. Cell Biol., 37, 726-730 (2005)]. Many of these are involved in cell cycle progression and apoptosis. It has been shown that PIM-1 acts as an anti-apoptotic factor via inactivation of the pro-apoptotic factor BAD (Bcl2 associated death promoter, an apoptosis initiator). This finding suggested a direct role of PIM-1 in preventing cell death, since the inactivation of BAD can enhance Bcl-2 activity and can thereby promote cell survival [Aho et al., FEBS Letters, 571, 43-49 (2004)]. PIM-1 has also been recognized as a positive regulator of cell cycle progression. PIM-1 binds and phosphorylates Cdc25A, which leads to an increase in its phosphatase activity and promotion of Gl/S transition [reviewed in Losman et al., JBC, 278, 4800-4805 (1999)]. In addition, the cyclin kinase inhibitor p21^{Waf} which inhibits Gl/S progression, was found to be inactivated by PIM-1 [Wang et al., Biochim. Biophys. Acta. 1593, 45-55 (2002)]. Furthermore, by means of phosphorylation, PIM-1 inactivates C-TAK1 and activates Cdc25C which results in acceleration of G2/M transition [Bachman et al., JBC, 279, 48319-48 (2004)].

PIM-1 appears to be an essential player in hematopoietic proliferation. Kinase active PIM-1 is required for the gp130-mediated STAT3 proliferation signal [Hirano et al., Oncogene 19, 2548-2556, (2000)]. PIM-1 is overexpressed or even mutated in a number of tumors and different types of tumor cell lines and leads to genomic instability. Fedorov, et al., concluded that a Phase III compound in development for treating leukemia, LY333′531, is a selective PIM-1 inhibitor. O. Fedorov, et al., PNAS 104(51), 20523-28 (December 2007). Evidence has been published to show that PIM-1 is involved in human tumors including prostate cancer, oral cancer, and Burkitt lymphoma (Gaidano & Dalla Faver, 1993). All these findings point to an important role of PIM-1 in the initiation and progression of human cancers, including various tumors and hematopoietic cancers, thus small molecule inhibitors of PIM-1 activity are a promising therapeutic strategy.

Additionally, PIM-2 and PIM-3 have overlapping functions with PIM-1 and inhibition of more than one isoform may provide additional therapeutic benefits. However, it is sometimes preferable for inhibitors of PIM to have little or no in vivo impact through their inhibition of various other kinases, since such effects are likely to cause side effects or unpredictable results. See, e.g., O. Fedorov, et al., PNAS 104(51), 20523-28 (December 2007), discussing the effects that non-specific kinase inhibitors can produce. Accordingly, in some embodiments, the invention provides compounds that are selective inhibitors of at least one of PIM-1, PIM-2, and PIM-3, or some combination of these, while having substantially less activity on certain other human kinases, as described further herein, although the compounds of Formula I are typically active on CK2 as well as one or more Pim proteins.

The implication of a role for PIM-3 in cancer was first suggested by transcriptional profiling experiments showing that PIM3 gene transcription was upregulated in EWS/ETS-induced malignant transformation of NIH 3T3 cells. These results were extended to show that PIM-3 is selectively expressed in human and mouse hepatocellular and pancreatic carcinomas but not in normal liver or pancreatic tissues. In addition, PIM-3 mRNA and protein are constitutively expressed in multiple human pancreatic and hepatocellular cancer cell lines.

The link between PIM-3 overexpression and a functional role in promoting tumorigenesis came from RNAi studies in human pancreatic and hepatocellular cancer cell lines overexpressing PIM-3. In these studies the ablation of endogenous PIM-3 protein promoted apoptosis of these cells. The molecular mechanism by which PIM-3 suppresses apoptosis is in part carried out through the modulation of phosphorylation of the pro-apoptotic protein BAD Similar to both PIM-1 & 2 which phosphorylate BAD protein, the knockdown of PIM-3 protein by siRNA results in a decrease in BAD phosphorylation at Serll2. Thus, similar to PIM-1 and 2, PIM-3 acts a suppressor of apoptosis in cancers of endodermal origin, e.g., pancreatic and liver cancers. Moreover, as conventional therapies in pancreatic cancer have a poor clinical outcome, PIM-3 could represent a new important molecular target towards successful control of this incurable disease.

At the 2008 AACR Annual Meeting, SuperGen announced that it has identified a lead PIM kinase inhibitor, SGI-1776, which causes tumor regression in acute myelogenous leukemia (AML) xenograft models (Abstract No. 4974). In an oral presentation entitled, “A potent small molecule PIM kinase inhibitor with activity in cell lines from hematological and solid malignancies,” Dr. Steven Warner detailed how scientists used SuperGen's CLIMB™ technology to build a model that allowed for the creation of small molecule PIM kinase inhibitors. SGI-1776 was identified as a potent and selective inhibitor of the PIM kinases, inducing apoptosis and cell cycle arrest, thereby causing a reduction in phospho-BAD levels and enhancement of mTOR inhibition in vitro. Most notably, SGI-1776 induced significant tumor regression in MV-4-11 (AML) and MOLM-13 (AML) xenograft models. This demonstrates that inhibitors of PIM kinases can be used to treat leukemias.

Fedorov, et al., in PNAS vol. 104(51), 20523-28, showed that a selective inhibitor of PIM-1 kinase (Ly5333′531) suppressed cell growth and induced cell death in leukemic cells from AML patients. PIM-3 has been shown to be expressed in pancreatic cancer cells, while it is not expressed in normal pancreas cells, demonstrating that it should be a good target for pancreatic cancer. Li, et al., Cancer Res. 66(13), 6741-47 (2006) Inhibitors of PIM kinases that are useful for treating certain types of cancers are described in PCT/US2008/012829.

Protein kinase CK2 (formerly called Casein kinase II, referred to herein as “CK2”) is a ubiquitous and highly conserved protein serine/threonine kinase. The holoenzyme is typically found in tetrameric complexes consisting of two catalytic (alpha and/or alpha′) subunits and two regulatory (beta) subunits. CK2 has a number of physiological targets and participates in a complex series of cellular functions including the maintenance of cell viability. The level of CK2 in normal cells is tightly regulated, and it has long been considered to play a role in cell growth and proliferation Inhibitors of CK2 that described as are useful for treating certain types of cancers are described in PCT/US2007/077464, PCT/US2008/074820, PCT/U52009/35609.

Both the prevalence and the importance of CK2 suggest it is an ancient enzyme on the evolutionary scale, as does an evolutionary analysis of its sequence; its longevity may explain why it has become important in so many biochemical processes, and why CK2 from hosts have even been co-opted by infectious pathogens (e.g., viruses, protozoa) as an integral part of their survival and life cycle biochemical systems. These same characteristics explain why inhibitors of CK2 are believed to be useful in a variety of medical treatments as discussed herein. Because it is central to many biological processes, as summarized by Guerra & Issinger, Curr. Med. Chem., 2008, 15:1870-1886, inhibitors of CK2, including the compounds described herein, should be useful in the treatment of a variety of diseases and disorders.

Cancerous cells show an elevation of CK2, and recent evidence suggests that CK2 exerts potent suppression of apoptosis in cells by protecting regulatory proteins from caspase-mediated degradation. The anti-apoptotic function of CK2 may contribute to its ability to participate in transformation and tumorigenesis. In particular, CK2 has been shown to be associated with acute and chronic myelogenous leukemia, lymphoma and multiple myeloma. In addition, enhanced CK2 activity has been observed in solid tumors of the colon, rectum and breast, squamous cell carcinomas of the lung and of the head and neck (SCCHN), adenocarcinomas of the lung, colon, rectum, kidney, breast, and prostate Inhibition of CK2 by a small molecule is reported to induce apoptosis of pancreatic cancer cells, and hepatocellular carcinoma cells (HegG2, Hep3, HeLa cancer cell lines); and CK2 inhibitors dramatically sensitized RMS (Rhabdomyosarcoma) tumors toward apoptosis induced by TRAIL. Thus an inhibitor of CK2 alone, or in combination with TRAIL or a ligand for the TRAIL receptor, would be useful to treat RMS, the most common soft-tissue sarcoma in children. In addition, elevated CK2 has been found to be highly correlated with aggressiveness of neoplasias, and treatment with a CK2 inhibitor of the invention should thus reduce tendency of benign lesions to advance into malignant ones, or for malignant ones to metastasize.

Unlike other kinases and signaling pathways, where mutations are often associated with structural changes that cause loss of regulatory control, increased CK2 activity level appears to be generally caused by upregulation or overexpression of the active protein rather than by changes that affect activation levels. Guerra and Issinger postulate this may be due to regulation by aggregation, since activity levels do not correlate well with mRNA levels. Excessive activity of CK2 has been shown in many cancers, including SCCHN tumors, lung tumors, breast tumors, and others. Id.

Elevated CK2 activity in colorectal carcinomas was shown to correlate with increased malignancy. Aberrant expression and activity of CK2 have been reported to promote increase nuclear levels of NF-kappaB in breast cancer cells. CK2 activity is markedly increased in patients with AML and CML during blast crisis, indicating that an inhibitor of CK2 should be particularly effective in these conditions. Multiple myeloma cell survival has been shown to rely on high activity of CK2, and inhibitors of CK2 were cytotoxic to MM cells. Similarly, a CK2 inhibitor inhibited growth of murine p190 lymphoma cells. Its interaction with Bcr/Abl has been reported to play an important role in proliferation of Bcr/Abl expressing cells, indicating inhibitors of CK2 may be useful in treatment of Bcr/Abl-positive leukemias Inhibitors of CK2 have been shown to inhibit progression of skin papillomas, prostate and breast cancer xenografts in mice, and to prolong survival of transgenic mice that express prostate-promoters. Id.

The role of CK2 in various non-cancer disease processes has been recently reviewed. See Guerra & Issinger, Curr. Med. Chem., 2008, 15:1870-1886. Increasing evidence indicates that CK2 is involved in critical diseases of the central nervous system, including, for example, Alzheimer's disease, Parkinson's disease, and rare neurodegenerative disorders such as Guam-Parkinson dementia, chromosome 18 deletion syndrome, progressive supranuclear palsy, Kuf's disease, or Pick's disease. It is suggested that selective CK2-mediated phosphorylation of tau proteins may be involved in progressive neurodegeneration of Alzheimer's. In addition, recent studies suggest that CK2 plays a role in memory impairment and brain ischemic, the latter effect apparently being mediated by CK2's regulatory effect on the PI3K survival pathways.

CK2 has also been shown to be involved in the modulation of inflammatory disorders, for example, acute or chronic inflammatory pain, glomerulonephritis, and autoimmune diseases, including, e.g., multiple sclerosis (MS), systemic lupus erythematosus, rheumatoid arthritis, and juvenile arthritis. It positively regulates the function of the serotonin 5-HT3 receptor channel, activates heme oxygenase type 2, and enhances the activity of neuronal nitric oxide synthase. A selective CK2 inhibitor was reported to strongly reduce pain response of mice when administered to spinal cord tissue prior to pain testing. It phosphorylates secretory type IIA phospholipase A2 from synovial fluid of RA patients, and modulates secretion of DEK (a nuclear DNA-binding protein), which is a proinflammatory molecule found in synovial fluid of patients with juvenile arthritis. Thus inhibition of CK2 is expected to control progression of inflammatory pathologies such as those described here, and the inhibitors disclosed herein have been shown to effectively treat pain in animal models.

Protein kinase CK2 has also been shown to play a role in disorders of the vascular system, such as, e.g., atherosclerosis, laminar shear stress, and hypoxia. CK2 has also been shown to play a role in disorders of skeletal muscle and bone tissue, such as cardiomyocyte hypertrophy, impaired insulin signaling and bone tissue mineralization. In one study, inhibitors of CK2 were effective at slowing angiogenesis induced by growth factor in cultured cells. Moreover, in a retinopathy model, a CK2 inhibitor combined with octreotide (a somatostatin analog) reduced neovascular tufts; thus the CK2 inhibitors described herein would be effective in combination with a somatostatin analog to treat retinopathy.

CK2 has also been shown to phosphorylate GSK, troponin and myosin light chain; thus it is important in skeletal muscle and bone tissue physiology, and is linked to diseases affecting muscle tissue.

Evidence suggests that CK2 is also involved in the development and life cycle regulation of protozoal parasites, such as, for example, Theileria parva, Trypanosoma cruzi, Leishmania donovani, Herpetomonas muscarum muscarum, Plasmodium falciparum, Trypanosoma brucei, Toxoplasma gondii and Schistosoma mansoni. Numerous studies have confirmed the role of CK2 in regulation of cellular motility of protozoan parasites, essential to invasion of host cells. Activation of CK2 or excessive activity of CK2 has been shown to occur in hosts infected with Leishmania donovani, Herpetomonas muscarum muscarum, Plasmodium falciparum, Trypanosoma brucei, Toxoplasma gondii and Schistosoma mansoni. Indeed, inhibition of CK2 has been shown to block infection by T. cruzi.

CK2 has also been shown to interact with and/or phosphorylate viral proteins associated with human immunodeficiency virus type 1 (HIV-1), human papilloma virus, and herpes simplex virus, in addition to other virus types (e.g. human cytomegalovirus, hepatitis C and B viruses, Borna disease virus, adenovirus, coxsackievirus, coronavirus, influenza, and varicella zoster virus). CK2 phosphorylates and activates HIV-1 reverse transcriptase and proteases in vitro and in vivo, and promotes pathogenicity of simian-human immunodeficiency virus (SHIV), a model for HIV Inhibitors of CK2 are thus able to reduce reduce pathogenic effects of a model of HIV infection. CK2 also phosphorylates numerous proteins in herpes simplex virus and numerous other viruses, and some evidence suggests viruses have adopted CK2 as a phosphorylating enzyme for their essential life cycle proteins. Inhibition of CK2 is thus expected to deter infection and progression of viral infections, which rely upon the host's CK2 for their own life cycles.

CK2 is unusual in the diversity of biological processes that it affects, and it differs from most kinases in other ways as well: it is constitutively active, it can use ATP or GTP, and it is elevated in most tumors and rapidly proliferating tissues. It also has unusual structural features that may distinguish it from most kinases, too, enabling its inhibitors to be highly specific for CK2 while many kinase inhibitors affect multiple kinases, increasing the likelihood of off-target effects, or variability between individual subjects. For all of these reasons, CK2 is a particularly interesting target for drug development, and the invention provides highly effective inhibitors of CK2 that are useful in treating a variety of different diseases and disorders mediated by or associated with excessive, aberrant or undesired levels of CK2 activity.

Because these protein kinases have important functions in biochemical pathways associated with cancer, immunological responses, and inflammation, and are also important in pathogenicity of certain microorganisms, inhibitors of their activity have many medicinal applications. The present invention provides novel compounds that inhibit CK2 or PIM or both, as well as compositions and methods of use utilizing these compounds. These compounds possess therapeutic utilities that are believed to derive from their activity as inhibitors of one or more of these protein kinases.

### DISCLOSURE OF THE INVENTION

The present invention in part provides chemical compounds having certain biological activities that include, but are not limited to, inhibiting cell proliferation, inhibiting angiogenesis, and modulating protein kinase activity. These molecules can modulate Pim kinase activity, and/or casein kinase 2 (CK2) activity, and in some cases may also modulate Fms-like tyrosine kinase 3 (Flt) activity, and thus affect biological functions that include but are not limited to, inhibiting gamma phosphate transfer from ATP to a protein or peptide substrate, inhibiting angiogenesis, inhibiting cell proliferation and inducing cell apoptosis, for example. The present invention also in part provides methods for preparing novel chemical compounds, and analogs thereof, and methods of using the foregoing. Also provided are compositions comprising the above-described molecules in combination with other agents, and methods for using such molecules in combination with other agents.

In one aspect, the invention provides compounds that inhibit at least one kinase selected from Pim-1, Pim-2, Pim-3, CK2, and Flt.

The compounds of the invention include compounds of Formula I:

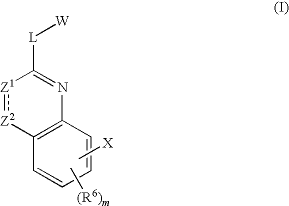

wherein:
Z¹ and Z² are independently selected from N, NR¹, C═V, and CR², provided Z¹ and Z² are not both NR¹;
   where R¹ and R² are independently selected from H, optionally substituted amino, optionally substituted alkoxy, optionally substituted alkyl, optionally substituted alkenyl, optionally substituted aryl, optionally substituted heteroaryl, optionally substituted heteroalkyl, optionally substituted heteroalkenyl, optionally substituted cycloalkyl, and optionally substituted heterocyclo alkyl;

C═V is C═CR¹R⁵ or C═NR¹;
and two R¹ groups, or R¹ and R² if both are present, can be taken together to form a 5-6 membered optionally substituted heterocyclic ring;

L is a linker selected from a bond, NR³, O, S, CR³R⁴, CR³R⁴—NR³, CR³R⁴—O—, and CR³R⁴—S;
where each R³, R⁴, R⁵, and R⁶ is independently H, or an optionally substituted member selected from the group consisting of C1-C8 alkyl, C2-C8 heteroalkyl, C2-C8 alkenyl, C2-C8 heteroalkenyl, C2-C8 alkynyl, C2-C8 heteroalkynyl, C1-C8 acyl, C2-C8 heteroacyl, C6-C10 aryl, C5-C12 heteroaryl, C7-C12 arylalkyl, and C6-C12 heteroarylalkyl group,
   or halo, OR, NR₂, NROR, NRNR₂, SR, SOR, SO₂R, SO₂NR₂, NRSO₂R, NRCONR₂, NRCSNR₂, NRC(═NR)NR₂, NRCOOR, NRCOR, CN, COOR, CONR₂, OOCR, COR, or NO₂,
   wherein each R is independently H or C1-C8 alkyl, C2-C8 heteroalkyl, C2-C8 alkenyl, C2-C8 heteroalkenyl, C2-C8 alkynyl, C2-C8 heteroalkynyl, C1-C8 acyl, C2-C8 heteroacyl, C6-C10 aryl, C5-C10 heteroaryl, C7-C12 arylalkyl, or C6-C12 heteroarylalkyl,
   and wherein two R on the same atom or on adjacent atoms can be linked to form a 3-8 membered ring, optionally containing one or more N, O or S;
      and each R group, and each ring formed by linking two R groups together, is optionally substituted with one or more substituents selected from halo, ═O, ═N—CN, ═N—OR′, ═NR′, OR′, NR′₂, SR′, SO₂R′, SO₂NR′₂, NR′SO₂R′, NR′CONR′₂, NR′CSNR′₂, NR′C(═NR′)NR′₂, NR′COOR′, NR′COR′, CN, COOR′, CONR′₂, OOCR′, COR′, and NO₂,
      wherein each R′ is independently H, C1-C6 alkyl, C2-C6 heteroalkyl, C1-C6 acyl, C2-C6 heteroacyl, C6-C10 aryl, C5-C10 heteroaryl, C7-12 arylalkyl, or C6-12 heteroarylalkyl, each of which is optionally substituted with one or more groups selected from halo, C1-C4 alkyl, C1-C4 heteroalkyl, C1-C6 acyl, C1-C6 heteroacyl, hydroxy, amino, and ═O;
         and wherein two R′ on the same atom or on adjacent atoms can be linked to form a 3-7 membered ring optionally containing up to three heteroatoms selected from N, O and S;
   and R³ and R⁴, when on the same atom or on adjacent connected atoms, can optionally be linked together to form a 3-8 membered cycloalkyl or heterocycloalkyl, which is optionally substituted;
W is alkyl, heteroalkyl, aryl, heteroaryl, cycloalkyl, or heterocyclyl, each of which can be optionally substituted;
X is a polar substituent;
and m is 0-2;

and the pharmaceutically acceptable salts of these compounds.

In another aspect, the compounds of the invention are compounds of Formula II:

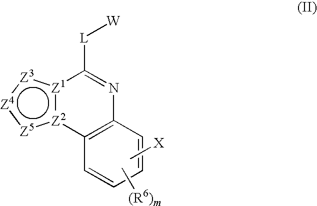

wherein Z¹ and Z² are each C, or one of Z¹ and Z² is N, the other of Z¹ and Z² is C;

Z³, Z⁴ and Z⁵ are independently selected from N, NR⁵, CR⁵ and O, provided not more than one of Z³-Z⁵ is O, and the ring containing Z³-Z⁵ is aromatic;

L is a linker selected from a bond, NR³, O, S, CR³R⁴, CR³R⁴—NR³, CR³R⁴—O—, and CR³R⁴—S;
where each R³, R⁴, R⁵, and R⁶ is independently H, or an optionally substituted member selected from the group consisting of C1-C8 alkyl, C2-C8 heteroalkyl, C2-C8 alkenyl, C2-C8 heteroalkenyl, C2-C8 alkynyl, C2-C8 heteroalkynyl, C1-C8 acyl, C2-C8 heteroacyl, C6-C10 aryl, C5-C12 heteroaryl, C7-C12 arylalkyl, and C6-C12 heteroarylalkyl group,
   or halo, OR, NR₂, NROR, NRNR₂, SR, SOR, SO₂R, SO₂NR₂, NRSO₂R, NRCONR₂, NRCSNR₂, NRC(═NR)NR₂, NRCOOR, NRCOR, CN, COOR, CONR₂, OOCR, COR, or NO₂,
   wherein each R is independently H or C1-C8 alkyl, C2-C8 heteroalkyl, C2-C8 alkenyl, C2-C8 heteroalkenyl, C2-C8 alkynyl, C2-C8 heteroalkynyl, C1-C8 acyl, C2-C8 heteroacyl, C6-C10 aryl, C5-C10 heteroaryl, C7-C12 arylalkyl, or C6-C12 heteroarylalkyl,
   and wherein two R on the same atom or on adjacent atoms can be linked to form a 3-8 membered ring, optionally containing one or more N, O or S;
      and each R group, and each ring formed by linking two R groups together, is optionally substituted with one or more substituents selected from halo, ═O, ═N—CN, ═N—OR′, ═NR′, OR′, NR′₂, SR′, SO₂R′, SO₂NR′₂, NR′SO₂R′, NR′CONR′₂, NR′CSNR′₂, NR′C(═NR′)NR′₂, NR′COOR′, NR′COR′, CN, COOR′, CONR′₂, OOCR′, COR′, and NO₂,
      wherein each R′ is independently H, C1-C6 alkyl, C2-C6 heteroalkyl, C1-C6 acyl, C2-C6 heteroacyl, C6-C10 aryl, C5-C10 heteroaryl, C7-12 arylalkyl, or C6-12 heteroarylalkyl, each of which is optionally substituted with one or more groups selected from halo, C1-C4 alkyl, C1-C4 heteroalkyl, C1-C6 acyl, C1-C6 heteroacyl, hydroxy, amino, and ═O;
         and wherein two R′ on the same atom or on adjacent atoms can be linked to form a 3-7 membered ring optionally containing up to three heteroatoms selected from N, O and S;
   and R³ and R⁴, when on the same atom or on adjacent connected atoms, can optionally be linked together to form a 3-8 membered cycloalkyl or heterocycloalkyl, which is optionally substituted;
W is alkyl, heteroalkyl, aryl, heteroaryl, cycloalkyl, or heterocyclyl, each of which can be substituted;
X is a polar substituent;
and m is 0-2;

or a pharmaceutically acceptable salt thereof.

In other aspects, the invention provides compositions comprising these compounds, and methods of using these compounds to treat various medical conditions, such as cancer, immunological disorders, pathogenic infections, inflammation, pain, angiogenesis-related disorders, and the like, as further described herein.

Also provided herein is a pharmaceutical composition comprising a compound of Formulae I or II described herein and at least one pharmaceutically acceptable carrier or excipient, or two or more pharmaceutically acceptable carriers and/or excipients. It is understood that the compounds of Formula I can include compounds of Formula Ia, and compounds of Formula II include compounds of Formula IIa. Pharmaceutical compositions of these compounds can be utilized in treatments described herein.

The compounds of the formulae disclosed herein may bind to and interact with kinases, and in one aspect the invention provides such a compound complexed with a kinase protein.

In certain embodiments, the protein is a CK2 protein, such as a CK2 protein comprising the amino acid sequence of SEQ ID NO: 1, 2 or 3 or a substantially identical variant thereof, for example. ‘Substantially identical’ means the sequence shares at least 90% homology to the specified sequence (SEQ ID NO: 1, 2 or 3), and preferably shares at least 90% sequence identity with the specified sequence.

| (NP_001886; casein kinase II alpha 1 subunit isoform a [Homo sapiens]) | |
|---|---|
| SEQ ID NO: 1 | |
| | msgpvpsrar vytdvnthrp reywdyeshv vewgnqddyq lvrklgrgky sevfeainit |
| | nnekvvvkil kpvkkkkikr eikilenlrg gpniitladi vkdpvsrtpa lvfehvnntd |
| 121 | fkqlyqtltd ydirfymyei lkaldychsm gimhrdvkph nvmidhehrk lrlidwglae |
| 181 | fyhpgqeynv rvasryfkgp ellvdyqmyd ysldmwslgc mlasmifrke pffhghdnyd |
| 241 | qlvriakvlg tedlydyidk ynieldprfn dilgrhsrkr werfvhsenq hlvspealdf |
| 301 | ldkllrydhq srltareame hpyfytvvkd qarmgsssmp ggstpvssan mmsgissvpt |
| 361 | psplgplags pviaaanplg mpvpaaagaq q |
| (NP_808227; casein kinase II alpha 1 subunit isoform a [Homo sapiens]) | |
| SEQ ID NO: 2 | |
| | msgpvpsrar vytdvnthrp reywdyeshv vewgnqddyq lvrklgrgky sevfeainit |
| | nnekvvvkil kpvkkkkikr eikilenlrg gpniitladi vkdpvsrtpa lvfehvnntd |
| 121 | fkqlyqtltd ydirfymyei lkaldychsm gimhrdvkph nvmidhehrk lrlidwglae |
| 181 | fyhpgqeynv rvasryfkgp ellvdyqmyd ysldmwslgc mlasmifrke pffhghdnyd |
| 241 | qlvriakvlg tedlydyidk ynieldprfn dilgrhsrkr werfvhsenq hlvspealdf |
| 301 | ldkllrydhq srltareame hpyfytvvkd qarmgsssmp ggstpvssan mmsgissvpt |
| 361 | psplgplags pviaaanplg mpvpaaagaq q |
| (NP_808228; casein kinase II alpha 1 subunit isoform b [Homo sapiens]) | |
| SEQ ID NO: 3 | |
| | myeilkaldy chsmgimhrd vkphnvmidh ehrklrlidw glaefyhpgq eynvrvasry |
| | fkgpellvdy qmydysldmw slgcmlasmi frkepffhgh dnydqlvria kvlgtedlyd |
| 121 | yidkynield prfndilgrh srkrwerfvh senqhlvspe aldfldkllr ydhqsrltar |
| 181 | eamehpyfyt vvkdqarmgs ssmpggstpv ssanmmsgis svptpsplgp lagspviaaa |
| 241 | nplgmpvpaa agaqq |

Substantially identical variants of these include proteins having at least 90% sequence homology with one of these, preferably at least 90% sequence identity; and having at least 50% of the level of in vitro kinase activity of the specified sequence under typical assay conditions.

In certain embodiments the protein is in a cell or in a cell-free system. The protein, the compound or the molecule in some embodiments is in association with a solid phase. In certain embodiments, the interaction between the compound and the protein is detected via a detectable label, where in some embodiments the protein comprises a detectable label and in certain embodiments the compound comprises a detectable label. The interaction between the compound and the protein sometimes is detected without a detectable label.

Also provided are methods for modulating the activity of a Pim protein, CK2 protein, or Flt protein which comprise contacting a system comprising the protein with a compound described herein in an amount effective for modulating the activity of the protein. In certain embodiments the activity of the protein is inhibited, and sometimes the protein is a CK2 protein, such as a CK2 protein comprising the amino acid sequence of SEQ ID NO: 1, 2 or 3 or a substantially identical variant thereof, for example. In other embodiments the protein is a Pim protein or a Flt protein. In certain embodiments, the system is a cell or tissue, and in other embodiments the system is a cell-free system. The protein or the compound may be in association with a solid phase in certain embodiments.

Provided also are methods for inhibiting cell proliferation, which comprise contacting cells with a compound described herein in an amount effective to inhibit proliferation of the cells. The cells sometimes are in a cell line, such as a cancer cell line (e.g., breast cancer, prostate cancer, pancreatic cancer, lung cancer, hemopoietic cancer, colorectal cancer, skin cancer, ovary cancer cell line), for example. In some embodiments, the cancer cell line is a breast cancer, prostate cancer or pancreatic cancer cell line. The cells sometimes are in a tissue, can be in a subject, at times are in a tumor, and sometimes are in a tumor in a subject. In certain embodiments, the method further comprises inducing cell apoptosis. Cells sometimes are from a subject having macular degeneration.

Also provided are methods for treating a condition related to aberrant cell proliferation, which comprise administering a compound described herein to a subject in need thereof in an amount effective to treat the cell proliferative condition. In certain embodiments the cell proliferative condition is a tumor-associated cancer. The cancer sometimes is of the breast, prostate, pancreas, lung, colorectum, skin, or ovary. In some embodiments, the cell proliferative condition is a non-tumor cancer, such as a hematopoietic cancer, for example. The cell proliferative condition is macular degeneration in some embodiments.

The invention also includes methods for treating cancer or an inflammatory disorder in a subject in need of such treatment, comprising: administering to the subject a therapeutically effective amount of a therapeutic agent useful for treating such disorder; and administering to the subject a molecule that inhibits Pim and/or CK2 in an amount that is effective to enhance a desired effect of the therapeutic agent. In certain embodiments, the molecule that inhibits CK2 and/or Pim is a compound of Formula I or II, including compounds of Formula Ia and IIa, or a pharmaceutically acceptable salt thereof. In certain embodiments, the desired effect of the therapeutic agent that is enhanced by the molecule that inhibits CK2 and/or Pim is an increase in apoptosis in at least one type of cell.

Provided also are methods for treating an immunological disorder, pain, or an inflammatory disorder in a subject in need of such treatment, comprising: administering to the subject a therapeutically effective amount of a therapeutic agent useful for treating such disorder; and administering to the subject a molecule that inhibits CK2, Pim or Flt in an amount that is effective to enhance a desired effect of the therapeutic agent. In certain embodiments, the molecule that inhibits CK2, Pim or Flt is a compound of Formula I or II as described herein, or a pharmaceutically acceptable salt thereof. In some embodiments, the molecule that inhibits CK2, Pim or Flt is a specific compound in one of the lists of compounds provided herein, or a pharmaceutically acceptable salt of one of these compounds. In some embodiments, the desired effect of the therapeutic agent that is enhanced by the molecule that inhibits CK2, Pim or Flt is a reduction in cell proliferation. In certain embodiments, the desired effect of the therapeutic agent that is enhanced by the molecule that inhibits CK2, Pim or Flt is an increase in apoptosis in at least one type of cell.

In some embodiments, the therapeutic agent and the molecule that inhibits CK2, Pim or Flt are administered at substantially the same time. The therapeutic agent and molecule that inhibits CK2, Pim or Flt sometimes are used concurrently by the subject. The therapeutic agent and the molecule that inhibits CK2, Pim or Flt are combined into one pharmaceutical composition in certain embodiments.

Also provided are compositions of matter comprising a compound described herein and an isolated protein. The protein sometimes is a CK2 protein, such as a CK2 protein comprising the amino acid sequence of SEQ ID NO: 1, 2 or 3 or a substantially identical variant thereof, for example. In some embodiments, the protein is a Pim protein. Certain compositions comprise a compound described herein in combination with a cell. The cell may be from a cell line, such as a cancer cell line. In the latter embodiments, the cancer cell line is sometimes a breast cancer, prostate cancer, pancreatic cancer, lung cancer, hematopoietic cancer, colorectal cancer, skin cancer, of ovary cancer cell line.

These and other embodiments of the invention are described in the description that follows.

### BRIEF DESCRIPTION OF THE DRAWINGS

FIG. 1A-D discloses specific compounds within the scope of the invention.

FIG. 2 provides synthetic routes a method for the preparation of compounds where the C-ring is a pyrrole.

FIG. 3 provides synthetic routes a method for the preparation of compounds wherein the C-ring is an imidazole.

FIG. 4 provides synthetic routes for the preparation and modification of the polar groups on the A-ring.

FIG. 5 provides a method for the preparation of quinolone intermediates where the C-ring is a pyrrole.

FIG. 6 provides a method for the preparation of compounds wherein the C-ring is an imidazole.

FIG. 7 provides a method a method for the preparation of compounds wherein the C-ring is a triazole.

FIG. 8 provides a method for the preparation of compounds wherein the C-ring is a triazolone.

FIG. 9 provides a method for the preparation of compounds wherein the C-ring is a pyrazole.

FIG. 10 provides a method for the preparation of triazoloquinoline compounds, wherein the C-ring is a 1,2,4 triazine.

FIG. 11 provides another method for the preparation of triazine-containing derivatives.

FIG. 12 provides a method for the preparation of compounds lacking a C-ring.

### MODES OF CARRYING OUT THE INVENTION

Compounds of Formula I and II exert biological activities that include, but are not limited to, inhibiting cell proliferation, reducing angiogenesis, preventing or reducing inflammatory responses and pain, and modulating certain immune responses. Compounds of these Formulae can modulate Pim activity, CK2 activity, or both, as demonstrated by the data herein. Such compounds therefore can be utilized in multiple applications by a person of ordinary skill in the art. For example, compounds described herein can be used, for example, for (i) modulation of Pim activity (e.g., PIM-1 activity), (ii) modulation of protein kinase activity (e.g., CK2 activity), (iii) modulation of cell proliferation, (iv) modulation of apoptosis, and (v) treatments of cell proliferation related disorders (e.g., administration alone or co-administration with another molecule).

For convenience, and without regard to standard nomenclature, when the position of groups on the bicyclic core portion of Formula I need to be described, the ring positions will be identified by number using the following numbering scheme:

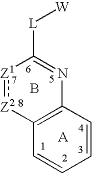

In this scheme, positions 1-4 are in the lower (phenyl) ring, and positions 5 (Nitrogen) through 8 are in the ring containing Z¹ and Z². So, for example, the position of the polar substituent X on the phenyl ring may be described as position 4 if that group is attached to the unsubstituted carbon adjacent to the phenyl ring carbon attached to N in the second ring. Also for convenience, the phenyl ring is labeled as ring A in this structure and throughout the application, while the ring containing Z¹ and Z² is labeled ‘B’ and can be referred to as ring B. The same relative numbering scheme will be used for other compounds that share the A and B ring bicyclic structure; where an additional ring is fused onto this bicyclic group at positions Z¹ and Z², that ring will be referred to as the C-ring herein.

The compounds of the invention often have ionizable groups so as to be capable of preparation as salts. In that case, wherever reference is made to the compound, it is understood in the art that a pharmaceutically acceptable salt may also be used. These salts may be acid addition salts involving inorganic or organic acids or the salts may, in the case of acidic forms of the compounds of the invention be prepared from inorganic or organic bases. Frequently, the compounds are prepared or used as pharmaceutically acceptable salts prepared as addition products of pharmaceutically acceptable acids or bases. Suitable pharmaceutically acceptable acids and bases are well-known in the art, such as hydrochloric, sulphuric, hydrobromic, acetic, lactic, citric, or tartaric acids for forming acid addition salts, and potassium hydroxide, sodium hydroxide, ammonium hydroxide, caffeine, various amines, and the like for forming basic salts. Methods for preparation of the appropriate salts are well-established in the art. In some cases, the compounds may contain both an acidic and a basic functional group, in which case they may have two ionized groups and yet have no net charge.

In some cases, the compounds of the invention contain one or more chiral centers. The invention includes each of the isolated stereoisomeric forms as well as mixtures of stereoisomers in varying degrees of chiral purity, including racemic mixtures. It also encompasses the various diastereomers and tautomers that can be formed. The compounds of the invention may also exist in more than one tautomeric form; the depiction herein of one tautomer is for convenience only, and is also understood to encompass other tautomers of the form shown.

As used herein, the terms “alkyl,” “alkenyl” and “alkynyl” include straight-chain, branched-chain and cyclic monovalent hydrocarbyl radicals, and combinations of these, which contain only C and H when they are unsubstituted. Examples include methyl, ethyl, isobutyl, cyclohexyl, cyclopentylethyl, 2-propenyl, 3-butynyl, and the like. The total number of carbon atoms in each such group is sometimes described herein, e.g., when the group can contain up to ten carbon atoms it can be represented as 1-10C or as C1-C10 or C1-10. When heteroatoms (N, O and S typically) are allowed to replace carbon atoms as in heteroalkyl groups, for example, the numbers describing the group, though still written as e.g. C1-C6, represent the sum of the number of carbon atoms in the group plus the number of such heteroatoms that are included as replacements for carbon atoms in the backbone of the ring or chain being described.

Typically, the alkyl, alkenyl and alkynyl substituents of the invention contain 1-10C (alkyl) or 2-10C (alkenyl or alkynyl). Preferably they contain 1-8C (alkyl) or 2-8C (alkenyl or alkynyl). Sometimes they contain 1-4C (alkyl) or 2-4C (alkenyl or alkynyl). A single group can include more than one type of multiple bond, or more than one multiple bond; such groups are included within the definition of the term “alkenyl” when they contain at least one carbon-carbon double bond, and are included within the term “alkynyl” when they contain at least one carbon-carbon triple bond.

“Optionally substituted” as used herein indicates that the particular group or groups being described may have no non-hydrogen substituents, or the group or groups may have one or more non-hydrogen substituents. If not otherwise specified, the total number of such substituents that may be present is equal to the number of H atoms present on the unsubstituted form of the group being described. Where an optional substituent is attached via a double bond, such as a carbonyl oxygen (═O), the group takes up two available valences, so the total number of substituents that may be included is reduced according to the number of available valences.

Alkyl, alkenyl and alkynyl groups are often optionally substituted to the extent that such substitution makes sense chemically. Typical substituents include, but are not limited to, halo, ═O, ═N—CN, ═N—OR, ═NR, OR, NR₂, SR, SO₂R, SO₂NR₂, NRSO₂R, NRCONR₂, NRCSNR₂, NRC(═NR)NR₂, NRCOOR, NRCOR, CN, C≡CR, COOR, CONR₂, OOCR, COR, and NO₂, wherein each R is independently H, C1-C8 alkyl, C2-C8 heteroalkyl, C1-C8 acyl, C2-C8 heteroacyl, C2-C8 alkenyl, C2-C8 heteroalkenyl, C2-C8 alkynyl, C2-C8 heteroalkynyl, C6-C10 aryl, or C5-C10 heteroaryl, and each R is optionally substituted with halo, ═O, ═N—CN, ═NR′, OR′, NR′₂, SR′, SO₂R′, SO₂NR′₂, NR′SO₂R′, NR′CONR′₂, NR′CSNR′₂, NR′C(═NR′)NR′₂, NR′COOR′, NR′COR′, CN, C≡CR′, COOR′, CONR′₂, OOCR′, COR′, and NO₂, wherein each R′ is independently H, C1-C8 alkyl, C2-C8 heteroalkyl, C1-C8 acyl, C2-C8 heteroacyl, C6-C10 aryl or C5-C10 heteroaryl. Alkyl, alkenyl and alkynyl groups can also be substituted by C1-C8 acyl, C2-C8 heteroacyl, C6-C10 aryl or C5-C10 heteroaryl, each of which can be substituted by the substituents that are appropriate for the particular group. Where two R or R′ are present on the same atom (e.g., NR₂ or NR′₂), or on adjacent atoms that are bonded together (e.g., —NR—C(O)R), the two R or R′ groups can be taken together with the atoms they are connected to form a 5-8 membered ring, which can be substituted with C1-C4 alkyl, C1-C4 acyl, halo, C1-C4 alkoxy, and the like, and can contain an additional heteroatom selected from N, O and S as a ring member.

“Acetylene” substituents are 2-10C alkynyl groups that are optionally substituted, and are of the formula —C≡C—R^{a}, wherein R^{a} is H or C1-C8 alkyl, C2-C8 heteroalkyl, C2-C8 alkenyl, C2-C8 heteroalkenyl, C2-C8 alkynyl, C2-C8 heteroalkynyl, C1-C8 acyl, C2-C8 heteroacyl, C6-C10 aryl, C5-C10 heteroaryl, C7-C12 arylalkyl, or C6-C12 heteroarylalkyl,
and each R^{a} group is optionally substituted with one or more substituents selected from halo, ═O, ═N—CN, ═N—OR′, ═NR′, OR′, NR′₂, SR′, SO₂R′, SO₂NR′₂, NR′SO₂R′, NR′CONR′₂, NR′CSNR′₂, NR′C(═NR′)NR′₂, NR′COOR′, NR′COR′, CN, COOR′, CONR′₂, OOCR′, COR′, and NO₂, wherein each R′ is independently H, C1-C6 alkyl, C2-C6 heteroalkyl, C1-C6 acyl, C2-C6 heteroacyl, C6-C10 aryl, C5-C10 heteroaryl, C7-12 arylalkyl, or C6-12 heteroarylalkyl, each of which is optionally substituted with one or more groups selected from halo, C1-C4 alkyl, C1-C4 heteroalkyl, C1-C6 acyl, C1-C6 heteroacyl, hydroxy, amino, and ═O; and wherein two R′ can be linked to form a 3-7 membered ring optionally containing up to three heteroatoms selected from N, O and S. In some embodiments, R^{a} of —C≡C—R^{a} is H or Me. Where two R or R′ are present on the same atom (e.g., NR₂ or NR′₂), or on adjacent atoms that are bonded together (e.g., —NR—C(O)R), the two R or R′ groups can be taken together with the atoms they are connected to form a 3-8 membered ring, preferably a 5-8 membered ring, which can be substituted with C1-C4 alkyl, C1-C4 acyl, halo, C1-C4 alkoxy, and the like, and can contain an additional heteroatom selected from N, O and S as a ring member.

“Heteroalkyl”, “heteroalkenyl”, and “heteroalkynyl” and the like are defined similarly to the corresponding hydrocarbyl (alkyl, alkenyl and alkynyl) groups, but the ‘hetero’ terms refer to groups that contain 1-3 O, S or N heteroatoms or combinations thereof within the backbone residue; thus at least one carbon atom of a corresponding alkyl, alkenyl, or alkynyl group is replaced by one of the specified heteroatoms to form a heteroalkyl, heteroalkenyl, or heteroalkynyl group. The typical and preferred sizes for heteroforms of alkyl, alkenyl and alkynyl groups are generally the same as for the corresponding hydrocarbyl groups, and the substituents that may be present on the heteroforms are the same as those described above for the hydrocarbyl groups. For reasons of chemical stability, it is also understood that, unless otherwise specified, such groups do not include more than two contiguous heteroatoms except where an oxo group is present on N or S as in a nitro or sulfonyl group.

While “alkyl” as used herein includes cycloalkyl and cycloalkylalkyl groups, the term “cycloalkyl” may be used herein to describe a carbocyclic non-aromatic group that is connected via a ring carbon atom, and “cycloalkylalkyl” may be used to describe a carbocyclic non-aromatic group that is connected to the molecule through an alkyl linker Similarly, “heterocyclyl” may be used to describe a non-aromatic cyclic group that contains at least one heteroatom as a ring member and that is connected to the molecule via a ring atom, which may be C or N; and “heterocyclylalkyl” may be used to describe such a group that is connected to another molecule through a linker. The sizes and substituents that are suitable for the cycloalkyl, cycloalkylalkyl, heterocyclyl, and heterocyclylalkyl groups are the same as those described above for alkyl groups. As used herein, these terms also include rings that contain a double bond or two, as long as the ring is not aromatic.

As used herein, “acyl” encompasses groups comprising an alkyl, alkenyl, alkynyl, aryl or arylalkyl radical attached at one of the two available valence positions of a carbonyl carbon atom, and heteroacyl refers to the corresponding groups wherein at least one carbon other than the carbonyl carbon has been replaced by a heteroatom chosen from N, O and S. Thus heteroacyl includes, for example, —C(═O)OR and —C(═O)NR₂ as well as —C(═O)-heteroaryl.

Acyl and heteroacyl groups are bonded to any group or molecule to which they are attached through the open valence of the carbonyl carbon atom. Typically, they are C1-C8 acyl groups, which include formyl, acetyl, pivaloyl, and benzoyl, and C2-C8 heteroacyl groups, which include methoxyacetyl, ethoxycarbonyl, and 4-pyridinoyl. The hydrocarbyl groups, aryl groups, and heteroforms of such groups that comprise an acyl or heteroacyl group can be substituted with the substituents described herein as generally suitable substituents for each of the corresponding component of the acyl or heteroacyl group.

“Aromatic” moiety or “aryl” moiety refers to a monocyclic or fused bicyclic moiety having the well-known characteristics of aromaticity; examples include phenyl and naphthyl. Similarly, “heteroaromatic” and “heteroaryl” refer to such monocyclic or fused bicyclic ring systems which contain as ring members one or more heteroatoms selected from O, S and N. The inclusion of a heteroatom permits aromaticity in 5-membered rings as well as 6-membered rings. Typical heteroaromatic systems include monocyclic C5-C6 aromatic groups such as pyridyl, pyrimidyl, pyrazinyl, thienyl, furanyl, pyrrolyl, pyrazolyl, thiazolyl, oxazolyl, and imidazolyl and the fused bicyclic moieties formed by fusing one of these monocyclic groups with a phenyl ring or with any of the heteroaromatic monocyclic groups to form a C8-C10 bicyclic group such as indolyl, benzimidazolyl, indazolyl, benzotriazolyl, isoquinolyl, quinolyl, benzothiazolyl, benzofuranyl, pyrazolopyridyl, quinazolinyl, quinoxalinyl, cinnolinyl, and the like. Any monocyclic or fused ring bicyclic system which has the characteristics of aromaticity in terms of electron distribution throughout the ring system is included in this definition. It also includes bicyclic groups where at least the ring which is directly attached to the remainder of the molecule has the characteristics of aromaticity. Typically, the ring systems contain 5-12 ring member atoms. Preferably the monocyclic heteroaryls contain 5-6 ring members, and the bicyclic heteroaryls contain 8-10 ring members.

Aryl and heteroaryl moieties may be substituted with a variety of substituents including C1-C8 alkyl, C2-C8 alkenyl, C2-C8 alkynyl, C5-C12 aryl, C1-C8 acyl, and heteroforms of these, each of which can itself be further substituted; other substituents for aryl and heteroaryl moieties include halo, OR, NR₂, SR, SO₂R, SO₂NR₂, NRSO₂R, NRCONR₂, NRCSNR₂, NRC(═NR)NR₂, NRCOOR, NRCOR, CN, C≡CR, COOR, CONR₂, OOCR, COR, and NO₂, wherein each R is independently H, C1-C8 alkyl, C2-C8 heteroalkyl, C2-C8 alkenyl, C2-C8 heteroalkenyl, C2-C8 alkynyl, C2-C8 heteroalkynyl, C6-C10 aryl, C5-C10 heteroaryl, C7-C12 arylalkyl, or C6-C12 heteroarylalkyl, and each R is optionally substituted as described above for alkyl groups. Where two R or R′ are present on the same atom (e.g., NR₂), or on adjacent atoms that are bonded together (e.g., —NR—C(O)R), the two R or R′ groups can be taken together with the atoms they are connected to form a 5-8 membered ring, which can be substituted with C1-C4 alkyl, C1-C4 acyl, halo, C1-C4 alkoxy, and the like, and can contain an additional heteroatom selected from N, O and S as a ring member.

The substituent groups on an aryl or heteroaryl group may of course be further substituted with the groups described herein as suitable for each type of such substituents or for each component of the substituent. Thus, for example, an arylalkyl substituent may be substituted on the aryl portion with substituents described herein as typical for aryl groups, and it may be further substituted on the alkyl portion with substituents described herein as typical or suitable for alkyl groups.

Similarly, “arylalkyl” and “heteroarylalkyl” refer to aromatic and heteroaromatic ring systems which are bonded to their attachment point through a linking group such as an alkylene, including substituted or unsubstituted, saturated or unsaturated, cyclic or acyclic linkers. Typically the linker is C1-C8 alkyl or a hetero form thereof. These linkers may also include a carbonyl group, thus making them able to provide substituents as an acyl or heteroacyl moiety. An aryl or heteroaryl ring in an arylalkyl or heteroarylalkyl group may be substituted with the same substituents described above for aryl groups. Preferably, an arylalkyl group includes a phenyl ring optionally substituted with the groups defined above for aryl groups and a C1-C4 alkylene that is unsubstituted or is substituted with one or two C1-C4 alkyl groups or heteroalkyl groups, where the alkyl or heteroalkyl groups can optionally cyclize to form a ring such as cyclopropane, dioxolane, or oxacyclopentane. Similarly, a heteroarylalkyl group preferably includes a C5-C6 monocyclic heteroaryl group that is optionally substituted with the groups described above as substituents typical on aryl groups and a C1-C4 alkylene that is unsubstituted or is substituted with one or two C1-C4 alkyl groups or heteroalkyl groups, or it includes an optionally substituted phenyl ring or C5-C6 monocyclic heteroaryl and a C1-C4 heteroalkylene that is unsubstituted or is substituted with one or two C1-C4 alkyl or heteroalkyl groups, where the alkyl or heteroalkyl groups can optionally cyclize to form a ring such as cyclopropane, dioxolane, or oxacyclopentane.

Where an arylalkyl or heteroarylalkyl group is described as optionally substituted, the substituents may be on either the alkyl or heteroalkyl portion or on the aryl or heteroaryl portion of the group. The substituents optionally present on the alkyl or heteroalkyl portion are the same as those described above for alkyl groups generally; the substituents optionally present on the aryl or heteroaryl portion are the same as those described above for aryl groups generally.

“Arylalkyl” groups as used herein are hydrocarbyl groups if they are unsubstituted, and are described by the total number of carbon atoms in the ring and alkylene or similar linker. Thus a benzyl group is a C7-arylalkyl group, and phenylethyl is a C8-arylalkyl.

“Heteroarylalkyl” as described above refers to a moiety comprising an aryl group that is attached through a linking group, and differs from “arylalkyl” in that at least one ring atom of the aryl moiety or one atom in the linking group is a heteroatom selected from N, O and S. The heteroarylalkyl groups are described herein according to the total number of atoms in the ring and linker combined, and they include aryl groups linked through a heteroalkyl linker; heteroaryl groups linked through a hydrocarbyl linker such as an alkylene; and heteroaryl groups linked through a heteroalkyl linker. Thus, for example, C7-heteroarylalkyl would include pyridylmethyl, phenoxy, and N-pyrrolylmethoxy.

“Alkylene” as used herein refers to a divalent hydrocarbyl group; because it is divalent, it can link two other groups together. Typically it refers to —(CH₂)ₙ— where n is 1-8 and preferably n is 1-4, though where specified, an alkylene can also be substituted by other groups, and can be of other lengths, and the open valences need not be at opposite ends of a chain. Thus —CH(Me)- and —C(Me)₂- may also be referred to as alkylenes, as can a cyclic group such as cyclopropan-1,1-diyl. Where an alkylene group is substituted, the substituents include those typically present on alkyl groups as described herein.

In general, any alkyl, alkenyl, alkynyl, acyl, or aryl or arylalkyl group or any heteroform of one of these groups that is contained in a substituent may itself optionally be substituted by additional substituents. The nature of these substituents is similar to those recited with regard to the primary substituents themselves if the substituents are not otherwise described. Thus, where an embodiment of, for example, R⁷ is alkyl, this alkyl may optionally be substituted by the remaining substituents listed as embodiments for R⁷ where this makes chemical sense, and where this does not undermine the size limit provided for the alkyl per se; e.g., alkyl substituted by alkyl or by alkenyl would simply extend the upper limit of carbon atoms for these embodiments, and is not included. However, alkyl substituted by aryl, amino, alkoxy, ═O, and the like would be included within the scope of the invention, and the atoms of these substituent groups are not counted in the number used to describe the alkyl, alkenyl, etc. group that is being described. Where no number of substituents is specified, each such alkyl, alkenyl, alkynyl, acyl, or aryl group may be substituted with a number of substituents according to its available valences; in particular, any of these groups may be substituted with fluorine atoms at any or all of its available valences, for example.

“Heteroform” as used herein refers to a derivative of a group such as an alkyl, aryl, or acyl, wherein at least one carbon atom of the designated carbocyclic group has been replaced by a heteroatom selected from N, O and S. Thus the heteroforms of alkyl, alkenyl, alkynyl, acyl, aryl, and arylalkyl are heteroalkyl, heteroalkenyl, heteroalkynyl, heteroacyl, heteroaryl, and heteroarylalkyl, respectively. It is understood that no more than two N, O or S atoms are ordinarily connected sequentially, except where an oxo group is attached to N or S to form a nitro or sulfonyl group.

“Halo”, as used herein includes fluoro, chloro, bromo and iodo. Fluoro and chloro are often preferred.

“Amino” as used herein refers to NH₂, but where an amino is described as “substituted” or “optionally substituted”, the term includes NR′R″ wherein each R′ and R″ is independently H, or is an alkyl, alkenyl, alkynyl, acyl, aryl, or arylalkyl group or a heteroform of one of these groups, and each of the alkyl, alkenyl, alkynyl, acyl, aryl, or arylalkyl groups or heteroforms of one of these groups is optionally substituted with the substituents described herein as suitable for the corresponding group. The term also includes forms wherein R′ and R″ on NR′R″ are linked together to form a 3-8 membered ring which may be saturated, unsaturated or aromatic and which contains 1-3 heteroatoms independently selected from N, O and S as ring members, and which is optionally substituted with the substituents described as suitable for alkyl groups or, if NR′R″ is an aromatic group, it is optionally substituted with the substituents described as typical for heteroaryl groups.

As used herein, the term “carbocycle” or “carbocyclic” refers to a cyclic compound containing only carbon atoms in the ring, whereas a “heterocycle” or “heterocyclic” refers to a cyclic compound comprising a heteroatom. The carbocyclic and heterocyclic structures encompass compounds having monocyclic, bicyclic or multiple ring systems. As used herein, unless otherwise specified, these terms include rings that are saturated, unsaturated, or aromatic.

As used herein, the term “heteroatom” refers to any atom that is not carbon or hydrogen, such as nitrogen, oxygen or sulfur. When it is part of the backbone or skeleton of a chain or ring, a heteroatom must be at least divalent, and will typically be selected from N, O, P, and S.

Illustrative examples of heterocycles include but are not limited to tetrahydrofuran, 1,3-dioxolane, 2,3-dihydrofuran, pyran, tetrahydropyran, benzofuran, isobenzofuran, 1,3-dihydro-isobenzofuran, isoxazole, 4,5-dihydroisoxazole, piperidine, pyrrolidine, pyrrolidin-2-one, pyrrole, pyridine, pyrimidine, octahydro-pyrrolo[3,4 b]pyridine, piperazine, pyrazine, morpholine, thiomorpholine, imidazole, imidazolidine 2,4-dione, 1,3-dihydrobenzimidazol-2-one, indole, thiazole, benzothiazole, thiadiazole, thiophene, tetrahydro thiophene 1,1-dioxide, diazepine, triazole, guanidine, diazabicyclo[2.2.1]heptane, 2,5-diazabicyclo[2.2.1]heptane, 2,3,4,4a,9,9a-hexahydro-1H-β-carboline, oxirane, oxetane, tetrahydropyran, dioxane, lactones, aziridine, azetidine, piperidine, lactams, and may also encompass heteroaryls. Other illustrative examples of heteroaryls include but are not limited to furan, pyrrole, pyridine, pyrimidine, imidazole, benzimidazole and triazole.

As used herein, the term “inorganic substituent” refers to substituents that do not contain carbon or contain carbon bound to elements other than hydrogen (e.g., elemental carbon, carbon monoxide, carbon dioxide, and carbonate). Examples of inorganic substituents include but are not limited to nitro, halogen, azido, cyano, sulfonyls, sulfinyls, sulfonates, phosphates, etc.

The term “polar substituent” as used herein refers to any substituent having an electric dipole, and optionally a dipole moment (e.g., an asymmetrical polar substituent has a dipole moment and a symmetrical polar substituent does not have a dipole moment). Polar substituents include substituents that accept or donate a hydrogen bond, and groups that would carry at least a partial positive or negative charge in aqueous solution at physiological pH levels. In certain embodiments, a polar substituent is one that can accept or donate electrons in a non-covalent hydrogen bond with another chemical moiety. In certain embodiments, a polar substituent is selected from a carboxy, a carboxy bioisostere or other acid-derived moiety that exists predominately as an anion at a pH of about 7 to 8 or higher. Other polar substituents include, but are not limited to, groups containing an OH or NH, an ether oxygen, an amine nitrogen, an oxidized sulfur or nitrogen, a carbonyl, a nitrile, and a nitrogen-containing or oxygen-containing heterocyclic ring whether aromatic or non-aromatic. In some embodiments, the polar substituent represented by R³ is a carboxylate or a carboxylate bioisostere.

“Carboxylate bioisostere” or “carboxy bioisostere” as used herein refers to a moiety that is expected to be negatively charged to a substantial degree at physiological pH. In certain embodiments, the carboxylate bioisostere is a moiety selected from the group consisting of:

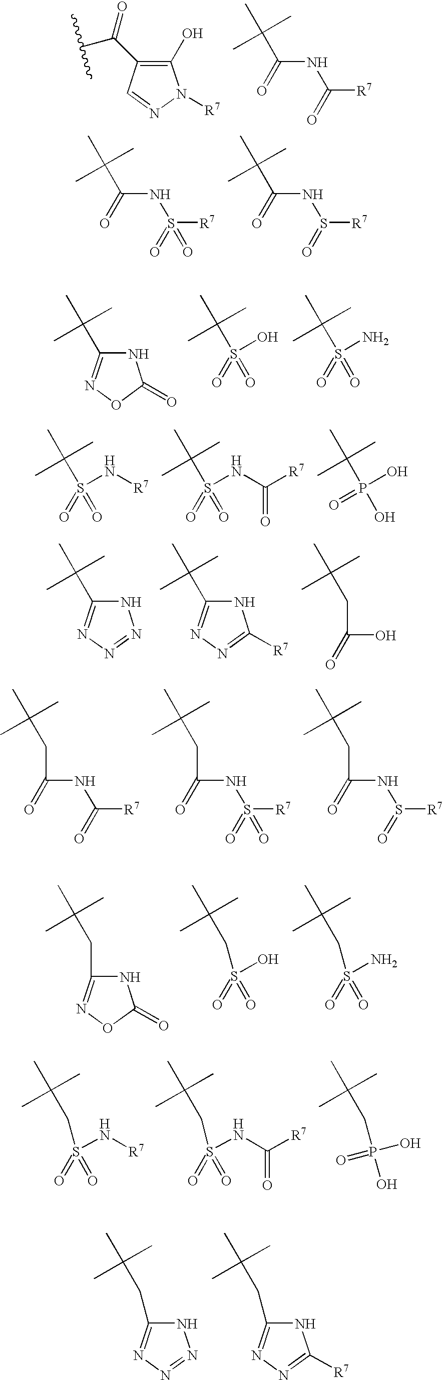

and salts of the foregoing, wherein each R⁷ is independently H or an optionally substituted member selected from the group consisting of C₁₋₁₀ alkyl, C₂₋₁₀ alkenyl, C₂₋₁₀ heteroalkyl, C₃₋₈ carbocyclic ring, and C₃₋₈ heterocyclic ring optionally fused to an additional optionally substituted carbocyclic or heterocyclic ring; or R⁷ is a C₁₋₁₀ alkyl, C₂₋₁₀ alkenyl, or C₂₋₁₀ heteroalkyl substituted with an optionally substituted C₃₋₈ carbocyclic ring or C₃₋₈ heterocyclic ring. In certain embodiments, the polar substituent is selected from the group consisting of carboxylic acid, carboxylic ester, carboxamide, tetrazole, triazole, carboxymethanesulfonamide, oxadiazole, oxothiadiazole, thiazole, aminothiazole and hydroxythiazole. In some embodiments of the compounds of Formula I or II, at least one polar substituent present is a carboxylic acid or a salt, or ester or a bioisostere thereof. In certain embodiments, at least one polar substituent present is a carboxylic acid-containing substituent or a salt, ester or bioisostere thereof. In the latter embodiments, the polar substituent may be a C1-C10 alkyl or C2-C10 alkenyl linked to a carboxylic acid (or salt, ester or bioisostere thereof), for example. In other embodiments, the polar substituent may comprise an aminoalkyl or hydroxyalkyl substituent.

The term ‘solgroup’ or ‘solubility-enhancing group’ as used herein refers to a molecular fragment selected for its ability to enhance physiological solubility of a compound that has otherwise relatively low solubility. Any substituent that can facilitate the dissolution of any particular molecule in water or any biological media can serve as a solubility-enhancing group. Examples of solubilizing groups are, but are not limited to: any substituent containing a group susceptible to being ionized in water at a pH range from 0 to 14; any ionizable group susceptible to form a salt; or any highly polar substituent, with a high dipolar moment and capable of forming strong interaction with molecules of water. Examples of solubilizing groups are, but are not limited to: substituted alkyl amines, substituted alkyl alcohols, alkyl ethers, aryl amines, pyridines, phenols, carboxylic acids, tetrazoles, sulfonamides, amides, sulfonylamides, sulfonic acids, sulfinic acids, phosphates, sulfonylureas.

Suitable groups for this purpose include, for example, groups of the formula -A-(CH₂)₀₋₄-G, where A is absent, O, or NR, where R is H or Me; and G can be a carboxy group, a carboxy bioisostere, hydroxy, phosphonate, sulfonate, or a group of the formula —NR^{y}₂ or P(O)(OR^{y})₂, where each R^{y} is independently H or a C1-C4 alkyl that can be substituted with one or more (typically up to three) of these groups: NH₂, OH, NHMe, NMe₂, OMe, halo, or ═O (carbonyl oxygen); and two R^{y} in one such group can be linked together to form a 5-7 membered ring, optionally containing an additional heteroatom (N, O or S) as a ring member, and optionally substituted with a C1-C4 alkyl, which can itself be substituted with one or more (typically up to three) of these groups: NH₂, OH, NHMe, NMe₂, OMe, halo, or ═O (carbonyl oxygen).

In one aspect, the invention provides compounds of Formula I:

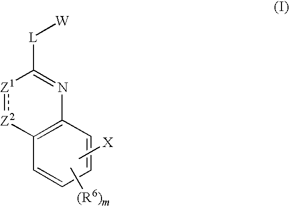

wherein:
Z¹ and Z² are independently selected from N, NR¹, C═V, and CR², provided Z¹ and Z² are not both NR¹;
   where R¹ and R² are independently selected from H, optionally substituted amino, optionally substituted alkoxy, optionally substituted alkyl, optionally substituted alkenyl, optionally substituted aryl, optionally substituted heteroaryl, optionally substituted heteroalkyl, optionally substituted heteroalkenyl, optionally substituted cycloalkyl, and optionally substituted heterocycloalkyl;

C═V is C═CR¹R⁵ or C═NR¹;
and two R¹ groups, or R¹ and R² if both are present, can be taken together to form a 5-6 membered optionally substituted heterocyclic ring;

L is a linker selected from a bond, NR³, O, S, CR³R⁴, CR³R⁴—NR³, CR³R⁴—O—, and CR³R⁴—S;
where each R³, R⁴, R⁵, and R⁶ is independently H, or an optionally substituted member selected from the group consisting of C1-C8 alkyl, C2-C8 heteroalkyl, C2-C8 alkenyl, C2-C8 heteroalkenyl, C2-C8 alkynyl, C2-C8 heteroalkynyl, C1-C8 acyl, C2-C8 heteroacyl, C6-C10 aryl, C5-C12 heteroaryl, C7-C12 arylalkyl, and C6-C12 heteroarylalkyl group,
   or halo, OR, NR₂, NROR, NRNR₂, SR, SOR, SO₂R, SO₂NR₂, NRSO₂R, NRCONR₂, NRCSNR₂, NRC(═NR)NR₂, NRCOOR, NRCOR, CN, COOR, CONR₂, OOCR, COR, or NO₂,
   wherein each R is independently H or C1-C8 alkyl, C2-C8 heteroalkyl, C2-C8 alkenyl, C2-C8 heteroalkenyl, C2-C8 alkynyl, C2-C8 heteroalkynyl, C1-C8 acyl, C2-C8 heteroacyl, C6-C10 aryl, C5-C10 heteroaryl, C7-C12 arylalkyl, or C6-C12 heteroarylalkyl,
   and wherein two R on the same atom or on adjacent atoms can be linked to form a 3-8 membered ring, optionally containing one or more N, O or S;
      and each R group, and each ring formed by linking two R groups together, is optionally substituted with one or more substituents selected from halo, ═O, ═N—CN, ═N—OR′, ═NR′, OR′, NR′₂, SR′, SO₂R′, SO₂NR′₂, NR′SO₂R′, NR′CONR′₂, NR′CSNR′₂, NR′C(═NR′)NR′₂, NR′COOR′, NR′COR′, CN, COOR′, CONR′₂, OOCR′, COR′, and NO₂,
      wherein each R′ is independently H, C1-C6 alkyl, C2-C6 heteroalkyl, C1-C6 acyl, C2-C6 heteroacyl, C6-C10 aryl, C5-C10 heteroaryl, C7-12 arylalkyl, or C6-12 heteroarylalkyl, each of which is optionally substituted with one or more groups selected from halo, C1-C4 alkyl, C1-C4 heteroalkyl, C1-C6 acyl, C1-C6 heteroacyl, hydroxy, amino, and ═O;
         and wherein two R′ on the same atom or on adjacent atoms can be linked to form a 3-7 membered ring optionally containing up to three heteroatoms selected from N, O and S;
   and R³ and R⁴, when on the same atom or on adjacent connected atoms, can optionally be linked together to form a 3-8 membered cycloalkyl or heterocycloalkyl, which is optionally substituted;
W is alkyl, heteroalkyl, aryl, heteroaryl, cycloalkyl, or heterocyclyl, each of which can be optionally substituted;
   X is a polar substituent;
   and m is 0-2;

or a pharmaceutically acceptable salt thereof.

In some embodiments of formula I, Z¹ is CR² and Z² is NR¹. In alternative embodiments of the foregoing compounds, Z¹ is NR¹ and Z² is CR². In each of these situations, R¹ and R² are both present. In other embodiments of the above-described compounds, one of Z¹ and Z² is C═V, and the other is NR¹. In this situation, two R¹ groups are present. Optionally, in such embodiments, the two R¹ groups or R² and R¹ can be linked together to form a 5-6 membered ring, which is fused to the B-ring of Formula I, providing a tricyclic ring system. The 5-6 membered fused ring may be saturated, unsaturated, or aromatic, and may have substituents described as suitable for the R¹ and R² groups. Typical fused rings formed by such cyclization are pyrroles, imidazoles, pyrazoles and triazoles, as well as furans, oxazoles and isoxazoles.

In some embodiments of formula I, Z¹ and Z² are both CR², or Z¹ and Z² are both N. In such embodiments, these groups do not form an additional fused ring.

In some embodiments, the compound has the formula Ia:

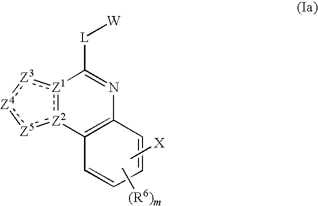

where one of Z¹ and Z² is N, and the other of these is C;
and Z³-Z⁵ are each independently CR⁵ or N,

and the dotted line bonds in the ring containing Z¹-Z⁵ indicate each of these bonds can be a single bond, double bond, or aromatic bond; and

W, L, X, R⁵, R⁶ and m are defined as for formula I.

In some embodiments of formula I and Ia, L is NH or NMe. In other embodiments, L can be NAc, where Ac represents a C1-C10 acyl group, i.e., L is a group of the formula N—C(═O)—R^{z}, where R^{z} is H or a C1-C9 optionally substituted alkyl group. These can serve as pro-drugs for compounds where L is NH. In still other embodiments, L is a bond; in these embodiments, W is often an aryl or heteroaryl, which is optionally substituted.

Note that in compounds of Formula I and in Formula II, L is a linker selected from a bond, NR³, O, S, CR³R⁴, CR³R⁴—NR³, CR³R⁴—O—, and CR³R⁴—S. Where L is a two-atom linker, it can be attached to the bicyclic ring system through either end, i.e., either the carbon atom or the heteroatom of CR³R⁴—NR³, CR³R⁴—O—, and CR³R⁴—S can be attached to the ring, and the other atom is attached to L.

In some embodiments of formula I and Ia, W is selected from optionally substituted alkyl, optionally substituted aryl, optionally substituted cycloalkyl, and optionally substituted heterocyclyl. For example, W can be an optionally substituted phenyl, pyridyl, pyrimidinyl, or pyrazinyl group; or a napthyl, indole; benzofuran, benzopyrazole, benzothiazole, quinoline, isoquinoline, quinazoline or quinoxaline group. In some embodiments, suitable substituents for these groups include, but are not limited to, halo, C1-C4 alkyl, C2-C4 alkenyl or alkynyl, CN, OMe, COOMe, COOEt, CONH₂, CF₃, and the like. In other embodiments, suitable substituent groups include halo, C1-C4 alkyl, C2-C4 alkenyl or alkynyl, C1-C4 alkoxy, C1-C4 alkylamino, C1-C4 alkoxyamino, CN, CF₃, carboalkoxy or carboxamido. Typically the aryl group is substituted by up to 2 of these groups; in preferred embodiments, when W is aryl or heteroaryl, it is unsubstituted, or it is substituted by 1 or 2 substituents.

In certain embodiments, W is an aryl ring substituted by a group of the formula —O—(CH₂)_{q}—NR^{x}₂ or —(CH₂)ᵣ—NR^{x}₂, where q is 1-4 and r is 0-4, and each R^{x} can be H or C1-C4 alkyl, and can be substituted, or where two R^{x} can optionally cyclize into a ring. In some embodiments, this group is of the formula —O—(CH₂)_{q}-Az or —(CH₂)ᵣ-Az, where q is 1-4 and r is 0-4, and Az represents an azacyclic group such as pyrrolidine, piperidine, morpholine, piperazine, thiomorpholine, pyrrole, and the like. In some embodiments, this group is —O—(CH₂)₁₋₃-Az or —(CH₂)₁₋₃-Az, where Az is 4-morpholinyl, 1-piperazinyl, 1-pyrrolidinyl, or 1-piperidinyl; —O—CH₂—CH₂-Az or —CH₂—CH₂-Az, where Az is 4-morpholinyl is one exemplary substituent for W, when W is substituted.

In some embodiments of formula I and Ia, W is optionally substituted phenyl, optionally substituted heterocyclyl, or C1-C4 alkyl substituted with at least one member selected from the group consisting of optionally substituted phenyl, optionally substituted heteroalkyl, optionally substituted heteroaryl, halo, and —NR″₂, where each R″ is independently H or optionally substituted C1-C6 alkyl, or two R″ taken together with the N to which they are attached can be linked together to form an optionally substituted 3-8 membered ring, which can contain another heteroatom selected from N, O and S as a ring member, and can be saturated, unsaturated or aromatic. In certain embodiments of such compounds, W comprises at least one group of the formula —(CH₂)ₚ—NR^{x}₂, where p is 1-4, R^{x} is independently at each occurrence H or optionally substituted alkyl; or two R^{x} taken together with the N to which they are attached can be linked together to form an optionally substituted 3-8 membered ring, which can contain another heteroatom selected from N, O and S as a ring member, and can be saturated, unsaturated or aromatic.

In some embodiments of formula I and Ia, X is selected from the group consisting of COOR⁹, C(O)NR⁹—OR⁹, triazole, tetrazole (preferably linked to the phenyl ring via the carbon atom of the tetrazole ring, i.e., 1,2,3,4-tetrazol-5-yl,), CN, imidazole, carboxylate, a carboxylate bioisostere,

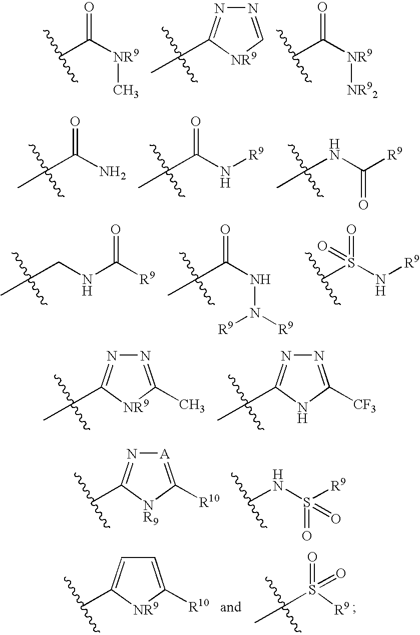

wherein each R⁹ is independently H or an optionally substituted member selected from the group consisting of alkyl, cycloalkyl, heterocyclyl, aryl, heteroaryl, arylalkyl, cycloalkylalkyl, heterocycloalkylalkyl, and heteroarylalkyl,
   and two R⁹ on the same or adjacent atoms can optionally be linked together to form an optionally substituted ring that can also contain an additional heteroatom selected from N, O and S as a ring member;
R¹⁰ is halo, CF₃, CN, SR, OR, NR₂, or R, where each R is independently H or optionally substituted C1-C6 alkyl, and two R on the same or adjacent atoms can optionally be linked together to form an optionally substituted ring that can also contain an additional heteroatom selected from N, O and S as a ring member;
and A is N or CR¹⁰.

In some embodiments of formula I and Ia, the polar substituent X is located at position 3 on the phenyl ring. In alternative embodiments, the polar substituent X is located at position 4 on the phenyl ring. In certain embodiments, the polar substituent is a carboxylic acid or a tetrazole, and is at position 3 or 4 on the phenyl ring.

In some embodiments of these compounds, the phenyl ring (i.e, the A ring) is substituted by up to three additional substituents, in addition to the polar substituent X. Suitable substituents for the phenyl are described above. In some such embodiments, these substituents are selected from halo, C1-C4 alkyl, C1-C4 haloalkyl, C1-C4 alkoxy, amino, C1-C4 alkylthio, and CN. In some embodiments, there is only one such substituent (i.e., m is 1), or there is no additional substituent besides the polar substituent X (i.e., m is 0).

In some embodiments of the above-described compounds, -L-W is selected from:

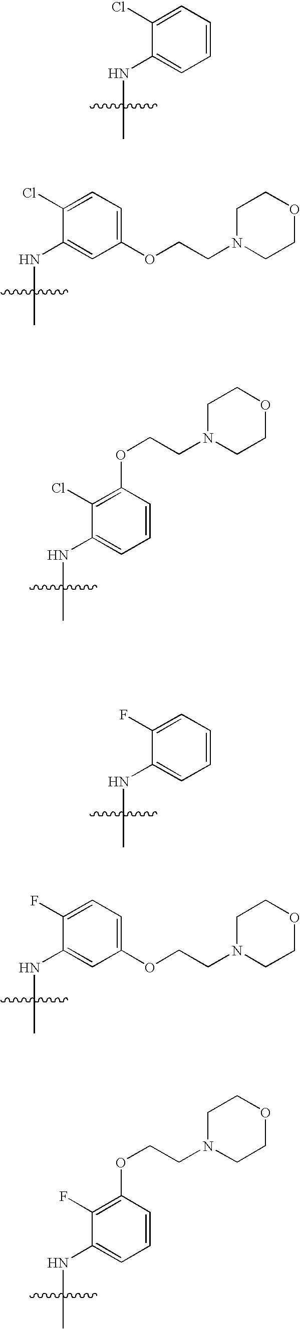

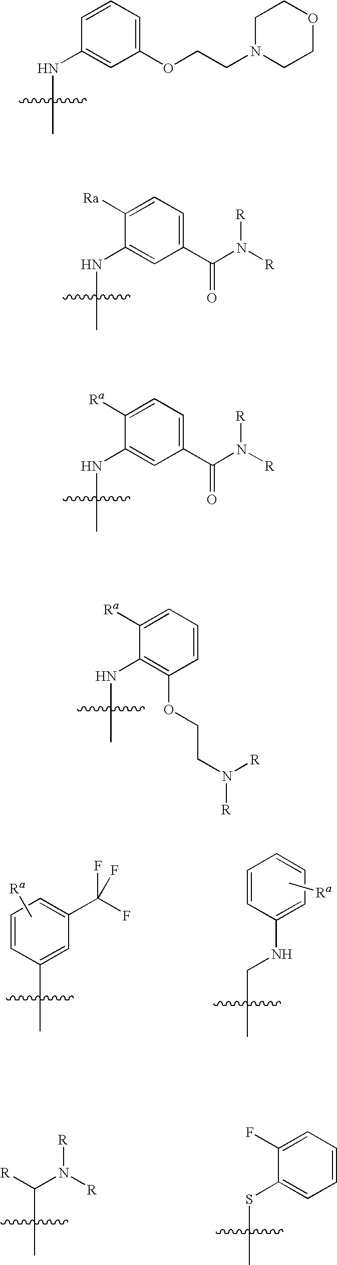

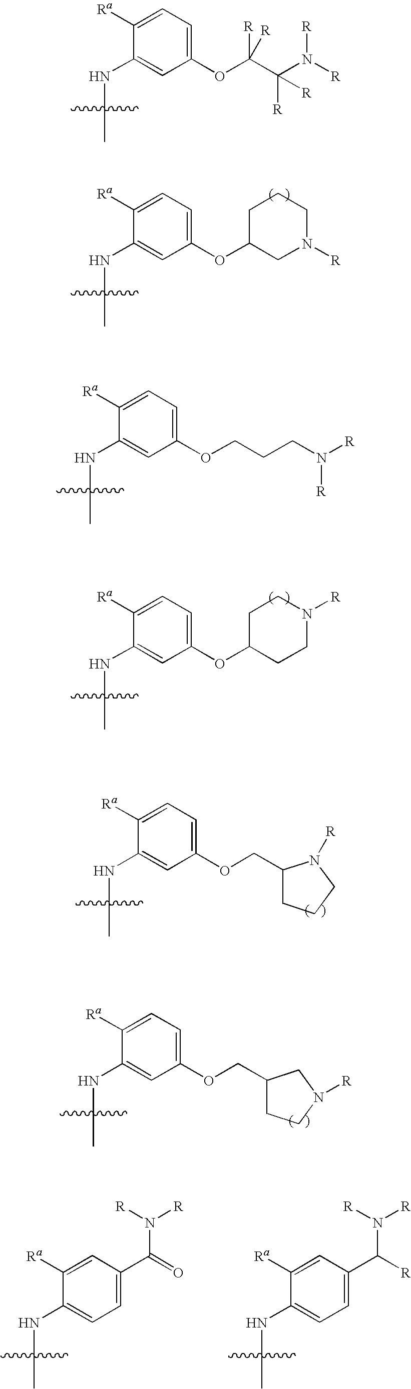

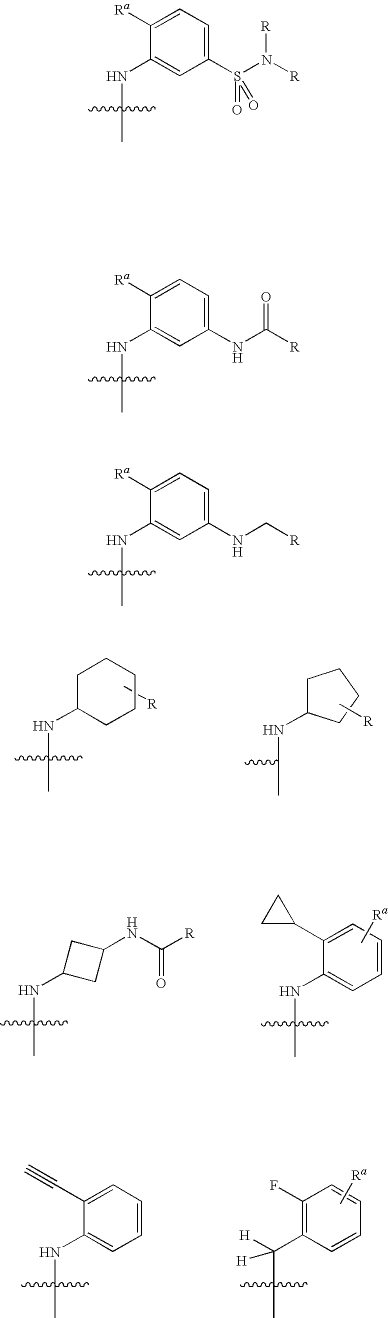

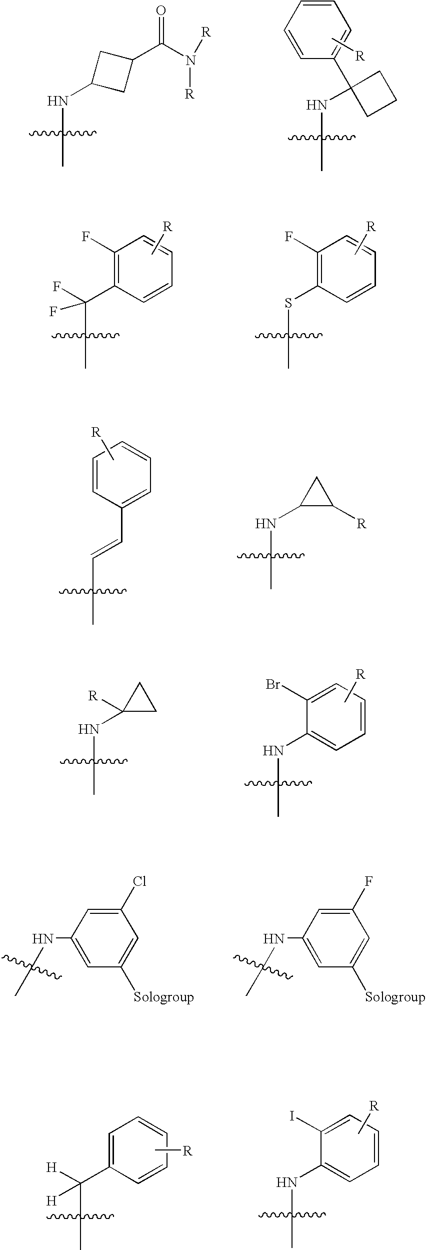

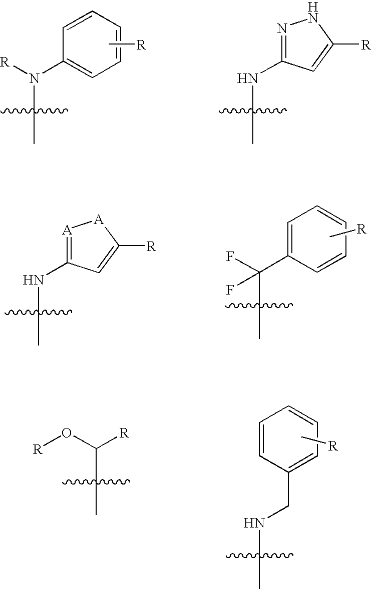

wherein each R^{a} is H, Cl or F;
each R is independently selected from halo, C1-C4 alkyl, C1-C4 alkoxy, and C1-C4 haloalkyl,
   and two R groups on the same or adjacent connected atoms can optionally be linked together to form a 3-8 membered ring;
each A is N or CR;

and each Solgroup is a solubility-enhancing group.

In frequent embodiments of formula (Ia), the ring containing Z¹-Z⁵ is an aromatic ring, such as a pyrrole, imidazole, pyrazole, triazole or tetrazole ring.

In some such embodiments, one of Z¹ and Z² is N and the other is C. In some such embodiments, Z¹ is N and Z² is C. In other such embodiments, Z¹ is C and Z² is N.

In some embodiments, one of Z¹ and Z² is N and the other is C. In some such embodiments, at least one of Z³-Z⁵ is N and the others are CR⁵. Sometimes, one of Z³-Z⁵ is N; sometimes, two of Z³-Z⁵ are N; and sometimes each of Z³-Z⁵ is N.

In certain embodiments of Formula Ia, Z² is N and Z¹ is C. In some such embodiments, at least one of Z³-Z⁵ is N and the others are CR⁵. In some of these embodiments, Z² and Z³ are N. In other of these embodiments, Z² and Z⁴ are N. In still other of these embodiments, Z² and Z⁵ are N. In other such embodiments, Z² is N, Z³ and Z⁴ are also N. In still other embodiments, Z² is N and Z³, Z⁴, Z⁵ are each CR⁵.

In other embodiments of Formula Ia, Z¹ is N and Z² is C. In some such embodiments, at least one of Z³-Z⁵ is N and the others are CR⁵. In some of these embodiments, Z¹ and Z⁴ are N. In other of these embodiments, Z¹ and Z⁵ are N. In still other of these embodiments, Z¹ and Z³ are N. In other such embodiments, Z¹ is N, Z⁴ and Z⁵ are also N. In still other embodiments, Z¹ is N and Z³, Z⁴, Z⁵ are each CR⁵.

In another aspect, the invention provides compounds of Formula II:

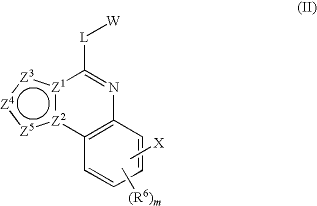

wherein Z¹ and Z² are each C, or one of Z¹ and Z² is N, the other of Z¹ and Z² is C;

Z³, Z⁴ and Z⁵ are independently selected from N, NR⁵, CR⁵ and O, provided not more than one of Z³-Z⁵ is O, and the ring containing Z³-Z⁵ is aromatic;

L is a linker selected from a bond, NR³, O, S, CR³R⁴, CR³R⁴—NR³, CR³R⁴—O—, and CR³R⁴—S;
where each R³, R⁴, R⁵, and R⁶ is independently H, or an optionally substituted member selected from the group consisting of C1-C8 alkyl, C2-C8 heteroalkyl, C2-C8 alkenyl, C2-C8 heteroalkenyl, C2-C8 alkynyl, C2-C8 heteroalkynyl, C1-C8 acyl, C2-C8 heteroacyl, C6-C10 aryl, C5-C12 heteroaryl, C7-C12 arylalkyl, and C6-C12 heteroarylalkyl group,
   or halo, OR, NR₂, NROR, NRNR₂, SR, SOR, SO₂R, SO₂NR₂, NRSO₂R, NRCONR₂, NRCSNR₂, NRC(═NR)NR₂, NRCOOR, NRCOR, CN, COOR, CONR₂, OOCR, COR, or NO₂,
   wherein each R is independently H or C1-C8 alkyl, C2-C8 heteroalkyl, C2-C8 alkenyl, C2-C8 heteroalkenyl, C2-C8 alkynyl, C2-C8 heteroalkynyl, C1-C8 acyl, C2-C8 heteroacyl, C6-C10 aryl, C5-C10 heteroaryl, C7-C12 arylalkyl, or C6-C12 heteroarylalkyl,
   and wherein two R on the same atom or on adjacent atoms can be linked to form a 3-8 membered ring, optionally containing one or more N, O or S;
      and each R group, and each ring formed by linking two R groups together, is optionally substituted with one or more substituents selected from halo, ═O, ═N—CN, ═NR′, OR′, NR′₂, SR′, SO₂R′, SO₂NR′₂, NR′SO₂R′, NR′CONR′₂, NR′CSNR′₂, NR′C(═NR′)NR′₂, NR′COOR′, NR′COR′, CN, COOR′, CONR′₂, OOCR′, COR′, and NO₂,
      wherein each R′ is independently H, C1-C6 alkyl, C2-C6 heteroalkyl, C1-C6 acyl, C2-C6 heteroacyl, C6-C10 aryl, C5-C10 heteroaryl, C7-12 arylalkyl, or C6-12 heteroarylalkyl, each of which is optionally substituted with one or more groups selected from halo, C1-C4 alkyl, C1-C4 heteroalkyl, C1-C6 acyl, C1-C6 heteroacyl, hydroxy, amino, and ═O;
         and wherein two R′ on the same atom or on adjacent atoms can be linked to form a 3-7 membered ring optionally containing up to three heteroatoms selected from N, O and S;
   and R³ and R⁴, when on the same atom or on adjacent connected atoms, can optionally be linked together to form a 3-8 membered cycloalkyl or heterocycloalkyl, which is optionally substituted;
W is alkyl, heteroalkyl, aryl, heteroaryl, cycloalkyl, or heterocyclyl, each of which can be substituted;
X is a polar substituent;
and m is 0-2;

or a pharmaceutically acceptable salt thereof.

In certain embodiments of these compounds of Formula II, Z¹ and Z² are both N.

In alternative embodiments, Z¹ and Z² are both C. In some of these embodiments, sometimes Z³-Z⁵ are each N or NR⁵. In other such embodiments, Z⁵ is CR⁵, and Z⁴ and Z³ are N or NR⁵. In still other embodiments, Z³ is CR⁵, and Z⁴ and Z⁵ are N or NR⁵. One of skill in the art will understand that in order to maintain aromaticity, no more than one of Z³-Z⁵ is NR⁵ in such embodiments. In still other embodiments of formula II, one of Z³-Z⁵ is O, and one of Z³-Z⁵ is N, and one of Z³-Z⁵ is CR⁵.

In compounds of formula (II), the ring containing Z¹-Z⁵ is an aromatic ring, such as a pyrrole, imidazole, pyrazole, triazole, tetrazole ring, furan, oxazole, or isoxazole ring.

In some embodiments of formula II, one of Z¹ and Z² is N and the other is C. In some such embodiments, Z¹ is N and Z² is C. In other such embodiments, Z¹ is C and Z² is N.

In some embodiments, one of Z¹ and Z² is N and the other is C, and at least one of Z³-Z⁵ is N and the others are CR⁵. Sometimes, one of Z³-Z⁵ is N; sometimes, two of Z³-Z⁵ are N; and sometimes each of Z³-Z⁵ is N.

In certain embodiments of Formula II, Z² is N and Z¹ is C. In some such embodiments, at least one of Z³-Z⁵ is N and the others are CR⁵. In some of these embodiments, Z² and Z³ are N. In other of these embodiments, Z² and Z⁴ are N. In still other of these embodiments, Z² and Z⁵ are N. In other such embodiments, Z² is N, Z³ and Z⁴ are also N. In still other embodiments, Z² is N and Z³, Z⁴, Z⁵ are each CR⁵.

In other embodiments of Formula II, Z¹ is N and Z² is C. In some such embodiments, at least one of Z³-Z⁵ is N and the others are CR⁵. In some of these embodiments, Z¹ and Z⁴ are N. In other of these embodiments, Z¹ and Z⁵ are N. In still other of these embodiments, Z¹ and Z³ are N. In other such embodiments, Z¹ is N, Z⁴ and Z⁵ are also N. In still other embodiments, Z¹ is N and Z³, Z⁴, Z⁵ are each CR⁵.

In certain embodiments, the compound of Formula II has structure of formula IIa:

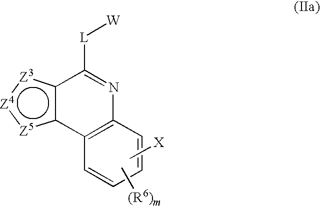

wherein one of Z³, Z⁴ and Z⁵ is either O or N, and the other two are selected from N and CR⁵, and the remaining groups are as defined for Formula II, or a pharmaceutically acceptable salt thereof.

In certain embodiments of Formula IIa, Z³ is O, while Z⁴ and Z⁵ are each CR⁵. In alternative embodiments, Z⁵ is O, while Z⁴ and Z³ are each CR⁵.

In certain embodiments of Formula II, the compound has structure of formula IIb or formula IIc:

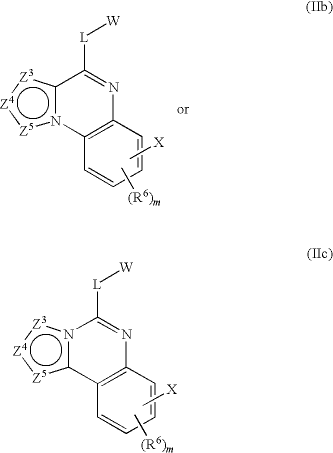

wherein Z³, Z⁴ and Z⁵ are independently selected from N and CR⁵, and the ring containing Z³-Z⁵ is aromatic;

L is a linker selected from a bond, NR³, O, S, CR³R⁴, CR³R⁴—NR³, CR³R⁴—O—, and CR³R⁴—S;
where each R³, R⁴, R⁵, and R⁶ is independently H, or an optionally substituted member selected from the group consisting of C1-C8 alkyl, C2-C8 heteroalkyl, C2-C8 alkenyl, C2-C8 heteroalkenyl, C2-C8 alkynyl, C2-C8 heteroalkynyl, C1-C8 acyl, C2-C8 heteroacyl, C6-C10 aryl, C5-C12 heteroaryl, C7-C12 arylalkyl, and C6-C12 heteroarylalkyl group,
   or halo, OR, NR₂, NROR, NRNR₂, SR, SOR, SO₂R, SO₂NR₂, NRSO₂R, NRCONR₂, NRCSNR₂, NRC(═NR)NR₂, NRCOOR, NRCOR, CN, COOR, CONR₂, OOCR, COR, or NO₂,
   wherein each R is independently H or C1-C8 alkyl, C2-C8 heteroalkyl, C2-C8 alkenyl, C2-C8 heteroalkenyl, C2-C8 alkynyl, C2-C8 heteroalkynyl, C1-C8 acyl, C2-C8 heteroacyl, C6-C10 aryl, C5-C10 heteroaryl, C7-C12 arylalkyl, or C6-C12 heteroarylalkyl,
   and wherein two R on the same atom or on adjacent atoms can be linked to form a 3-8 membered ring, optionally containing one or more N, O or S;
      and each R group, and each ring formed by linking two R groups together, is optionally substituted with one or more substituents selected from halo, ═O, ═N—CN, ═N—OR′, ═NR′, OR′, NR′₂, SR′, SO₂R′, SO₂NR′₂, NR′SO₂R′, NR′CONR′₂, NR′CSNR′₂, NR′C(═NR′)NR′₂, NR′COOR′, NR′COR′, CN, COOR′, CONR′₂, OOCR′, COR′, and NO₂,
      wherein each R′ is independently H, C1-C6 alkyl, C2-C6 heteroalkyl, C1-C6 acyl, C2-C6 heteroacyl, C6-C10 aryl, C5-C10 heteroaryl, C7-12 arylalkyl, or C6-12 heteroarylalkyl, each of which is optionally substituted with one or more groups selected from halo, C1-C4 alkyl, C1-C4 heteroalkyl, C1-C6 acyl, C1-C6 heteroacyl, hydroxy, amino, and ═O;
         and wherein two R′ on the same atom or on adjacent atoms can be linked to form a 3-7 membered ring optionally containing up to three heteroatoms selected from N, O and S;
   and R³ and R⁴, when on the same atom or on adjacent connected atoms, can optionally be linked together to form a 3-8 membered cycloalkyl or heterocycloalkyl, which is optionally substituted;
W is alkyl, heteroalkyl, aryl, heteroaryl, cycloalkyl, or heterocyclyl, each of which can be substituted;
X is a polar substituent;
and m is 0-2;

or a pharmaceutically acceptable salt thereof.

In compounds of formula IIb, 0, 1, 2 or 3 of Z³-Z⁵ is N and the others are CR⁵. In some such embodiments, each of Z³-Z⁵ is CR⁵. In other embodiments, at least one of Z³-Z⁵ is N and the others are CR⁵. Sometimes, one of Z³-Z⁵ is N; sometimes, two of Z³-Z⁵ are N; and sometimes each of Z³-Z⁵ is N.

In some embodiments of formula IIb, one of Z³-Z⁵ is N and the others are CR⁵. In one such embodiment, Z⁴ is N, and Z³ and Z⁵ are CR⁵. In another such embodiment, Z³ is N, and Z⁴ and Z⁵ are CR⁵. In another such embodiment, Z⁵ is N, and Z³ and Z⁴ are CR⁵. In another embodiment of formula IIb, two of Z³-Z⁵ are N. In one such embodiment, Z³ and Z⁴ are N and Z⁵ is CR⁵. In still other embodiments of formula IIb, Z³, Z⁴, Z⁵ are each CR⁵.

In compounds of formula IIc, 0, 1, 2 or 3 of Z³-Z⁵ is N and the others are CR⁵. In some such embodiments, each of Z³-Z⁵ is CR⁵. In other embodiments, at least one of Z³-Z⁵ is N and the others are CR⁵. Sometimes, one of Z³-Z⁵ is N; sometimes, two of Z³-Z⁵ are N; and sometimes each of Z³-Z⁵ is N.

In compounds of formula IIc, 0, 1, 2 or 3 of Z³-Z⁵ is N and the others are CR⁵. In some such embodiments, each of Z³-Z⁵ is CR⁵. In other such embodiments, one of Z³-Z⁵ is N and the others are CR⁵. In other embodiments, two of Z³-Z⁵ are N and the other is CR⁵. In other embodiments, each of Z³-Z⁵ is N.

In some embodiments of formula IIc, one of Z³-Z⁵ is N and the others are CR⁵. In one such embodiment, Z⁴ is N, and Z³ and Z⁵ are CR⁵. In another such embodiment, Z⁵ is N, and Z⁴ and Z⁵ are CR⁵. In another such embodiment, Z³ is N, and Z³ and Z⁴ are CR⁵. In another embodiment of formula IIc, two of Z³-Z⁵ are N. In one such embodiment, Z⁴ and Z⁵ are N and Z³ is CR⁵. In still other embodiments of formula IIb, Z³, Z⁴, Z⁵ are each CR⁵.

In some embodiments of the compounds of formula II, IIa, IIb and IIc, W is selected from optionally substituted alkyl, optionally substituted aryl, optionally substituted cycloalkyl, and optionally substituted heterocyclyl. For example, W can be an optionally substituted phenyl, pyridyl, pyrimidinyl, or pyrazinyl group; or a napthyl, indole; benzofuran, benzopyrazole, benzothiazole, quinoline, isoquinoline, quinazoline or quinoxaline group. Suitable substituents for these groups include, but are not limited to, halo, C1-C4 alkyl, C2-C4alkenyl or alkynyl, CN, OMe, COOMe, COOEt, CONH₂, CF₃, and the like, and typically the aryl group is substituted by up to 2 of these groups; in preferred embodiments, when W is aryl or heteroaryl, it is unsubstituted, or it is substituted by 1 or 2 substituents.

In certain embodiments of the compounds of formula II, IIa, IIb and IIc, W is selected from optionally substituted alkyl, optionally substituted aryl, optionally substituted heterocyclyl, and optionally substituted cycloalkyl.

In some embodiments of these compounds, L is NH or NMe. In other embodiments, L can be NAc, where Ac represents a C1-C10 acyl group, i.e., L is a group of the formula N—C(═O)—R^{z}, where R^{z} is H or a C1-C9 optionally substituted alkyl group. These can serve as pro-drugs for compounds where L is NH.

In certain embodiments of the foregoing compounds of formula II, IIa, IIb and IIc, L is NH or NMe, and W is optionally substituted phenyl, optionally substituted heterocyclyl, or C1-C4 alkyl substituted with at least one member selected from the group consisting of optionally substituted phenyl, optionally substituted heteroalkyl, optionally substituted heteroaryl, halo, and —NR″₂, where each R″ is independently H or optionally substituted C1-C6 alkyl, or two R″ taken together with the N to which they are attached can be linked together to form an optionally substituted 3-8 membered ring, which can contain another heteroatom selected from N, O and S as a ring member, and can be saturated, unsaturated or aromatic.

In certain embodiments of the compounds formula II, IIa, IIb and IIc, W comprises at least one group of the formula —(CH₂)ₚ—NR^{x}₂, where p is 1-4, R^{x} is independently at each occurrence H or optionally substituted alkyl; or two R^{x} taken together with the N to which they are attached can be linked together to form an optionally substituted 3-8 membered ring, which can contain another heteroatom selected from N, O and S as a ring member, and can be saturated, unsaturated or aromatic.

In certain embodiments of the formula II, IIa, IIb and IIc, X is selected from the group consisting of COOR⁹, C(O)NR⁹—OR⁹, triazole, tetrazole (e.g., 1,2,3,4-tetrazol-5-yl), CN, imidazole, carboxylate, a carboxylate bioisostere,

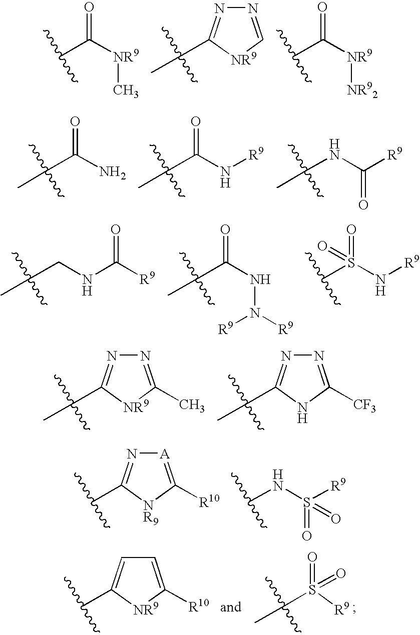

wherein each R⁹ is independently H or an optionally substituted member selected from the group consisting of alkyl, cycloalkyl, heterocyclyl, aryl, heteroaryl, arylalkyl, cycloalkylalkyl, heterocycloalkylalkyl, and heteroarylalkyl,
   and two R⁹ on the same or adjacent atoms can optionally be linked together to form an optionally substituted ring that can also contain an additional heteroatom selected from N, O and S as a ring member;
R¹⁰ is halo, CF₃, CN, SR, OR, NR₂, or R, where each R is independently H or optionally substituted C1-C6 alkyl, and two R on the same or adjacent atoms can optionally be linked together to form an optionally substituted ring that can also contain an additional heteroatom selected from N, O and S as a ring member;
and A is N or CR¹⁰.

In certain embodiments of the compounds formula II, IIa, IIb and IIc, the polar substituent X is located at position 3 on the phenyl ring. In alternative embodiments, the polar substituent X is located at position 4 on the phenyl ring. In certain embodiments, the polar substituent is a carboxylic acid or a C-linked tetrazole, and is at position 3 or 4 on the phenyl ring.

In some embodiments of these compounds, the phenyl ring is substituted by up to three additional substituents, in addition to the polar substituent X. Suitable substituents for the phenyl are described above. In some embodiments, these substituents are selected from halo, C1-C4 alkyl, C1-C4 haloalkyl, C1-C4 alkoxy, amino, C1-C4 alkylthio, and CN. In some embodiments, there is only one such substituent (i.e., m is 1), or there is no additional substituent besides the polar substituent X, i.e., m is 0.

In certain embodiments of the compounds of formula II, IIa, IIb and IIc, -L-W is selected from:

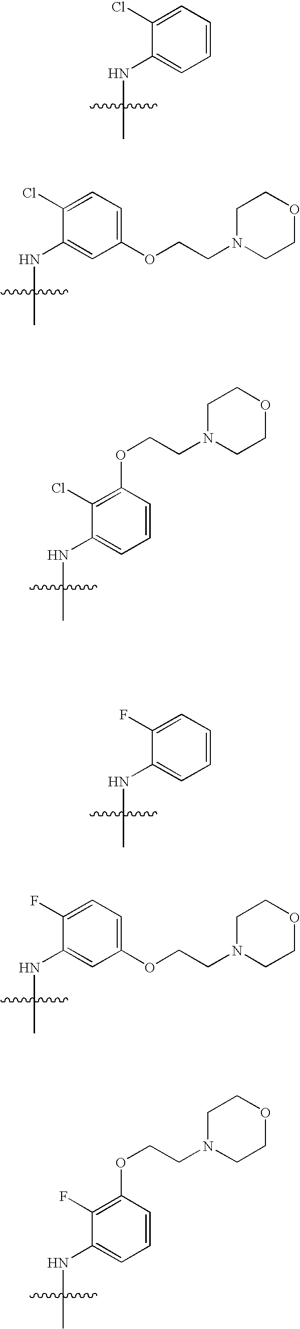

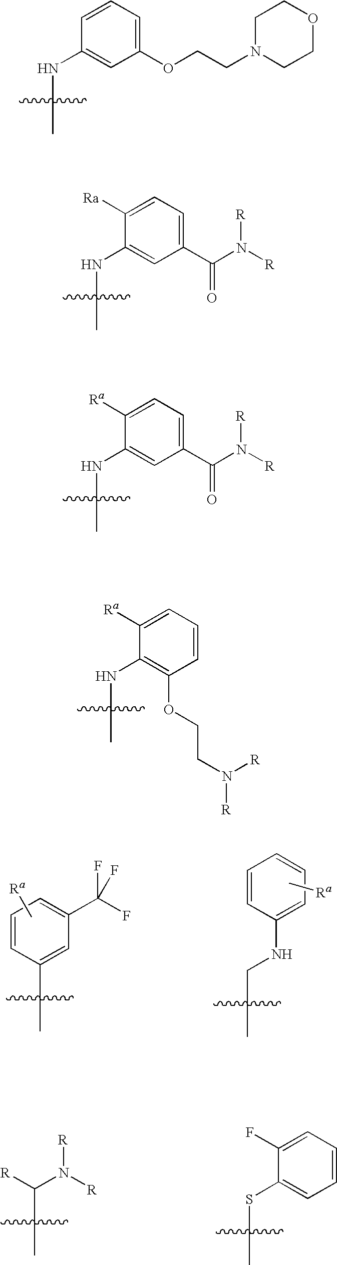

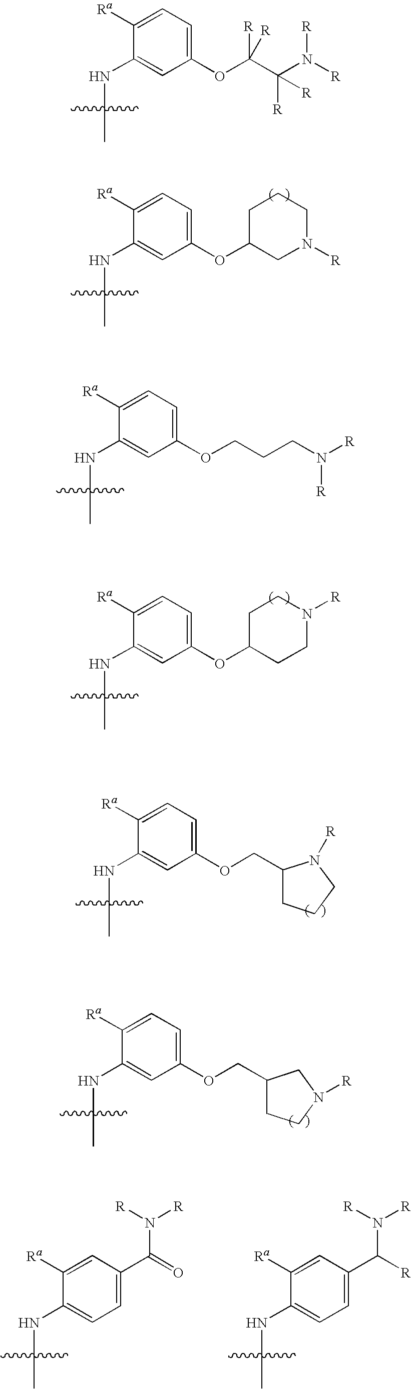

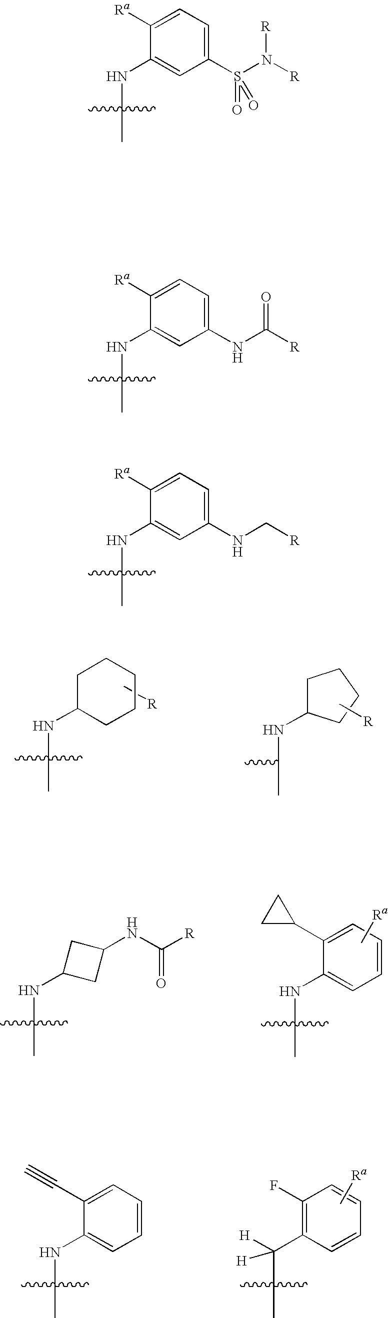

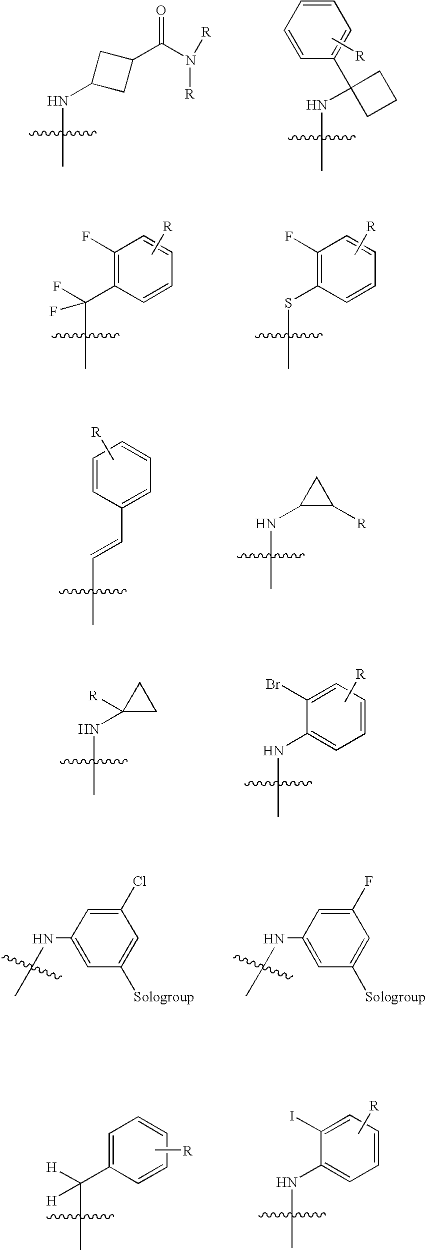

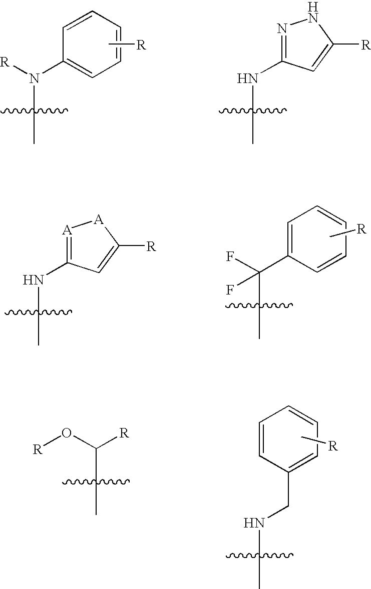

wherein each R^{a} is H, Cl or F;
each R is independently selected from halo, C1-C4 alkyl, C1-C4 alkoxy, and C1-C4 haloalkyl,
   and two R groups on the same or adjacent connected atoms can optionally be linked together to form a 3-8 membered ring;
each A is N or CR;

and each Solgroup is a solubility-enhancing group.

In certain embodiments of the invention, the compound is any of the species disclosed herein; or a pharmaceutically acceptable salt thereof.

It will be understood that embodiments described herein as useful for compounds of formula I also are applicable to compounds of formula Ia, and that embodiments described herein as useful for compounds of formula II also are applicable to compounds of formula IIa, IIb and IIc.

In compounds of the formulae disclosed herein, at least one polar substituent X may be at any position on the phenyl ring (i.e., the A-ring) in Formula I or II, and the ring may include one, two, three or four polar substituents. In certain embodiments, there is one polar group X, and each R⁶ is H, or up to two R⁶ are substituents described herein other than H, such as, for example only, Me, Et, halo (especially F or Cl), MeO, CF₃, CONH₂, or CN. A polar group can be at any position on the phenyl ring; in some embodiments, the phenyl ring is selected from the following options, which are oriented to match the orientation shown in Formula I herein, and depict the position of the polar substituent X:

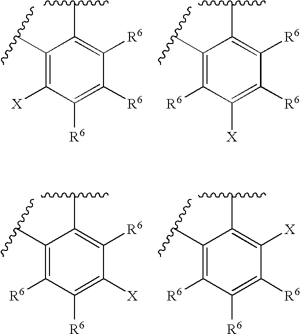

where X is a polar substituent and each R⁶ is independently selected from R⁶ substituents, as defined above with respect to compounds of Formula I, Ia, II, IIa, IIb and IIc.

L in Formula I, Ia, II, IIa, IIb and IIc can be a bond, or a 1-2 atom linker, including —N(R³)—, —O—, —S—, —CH₂—N(R³)—, —N(R³)—CH₂—, —O—CH₂—, —CH₂—O—, —CH₂—S—, —S—CH₂—, —CMe₂N(R³)—, —CMe₂-O—, —N(R³)—CMe₂, —O—CMe₂-, and the like. In certain embodiments, L is selected from a bond, NH, NMe, and —CH₂—N(R³)— or —N(R³)—CH₂—, where R³ is H or Me.

W in Formula I, Ia, II, IIa, IIb and IIc can be aryl (e.g., phenyl), heterocyclic (e.g., pyrrolidine, piperidine, morpholine, piperazine, thiomorpholine), or heteroaryl (e.g., pyrrole, pyridine, pyrazine, pyrimidine, furan, thiophene, thiazole, isothiazole, thiadiazole, oxazole, isoxazole, imidazole, pyrazole, triazole, triazine, tetrazole and the like, each of which can be substituted. In some embodiments, it is selected from phenyl, pyrrolidine, piperidine, piperazine, morpholine, and the like.

In some embodiments of formula II, IIa, IIb and Ic, W is selected from optionally substituted alkyl, optionally substituted aryl, optionally substituted cycloalkyl, and optionally substituted heterocyclyl. For example, W can be an optionally substituted phenyl, pyridyl, pyrimidinyl, or pyrazinyl group; or a napthyl, indole; benzofuran, benzopyrazole, benzothiazole, quinoline, isoquinoline, quinazoline or quinoxaline group. In some embodiments, suitable substituents for these groups include, but are not limited to, halo, C1-C4 alkyl, C2-C4 alkenyl or alkynyl, CN, OMe, COOMe, COOEt, CONH₂, CF₃, and the like. In other embodiments, suitable substituent groups include halo, C1-C4 alkyl, C2-C4 alkenyl or alkynyl, C1-C4 alkoxy, C1-C4 alkylamino, C1-C4 alkoxyamino, CN, CF₃, carboalkoxy or carboxamido. Typically the aryl group is substituted by up to 2 of these groups; in preferred embodiments, when W is aryl or heteroaryl, it is unsubstituted, or it is substituted by 1 or 2 substituents.

In certain embodiments, W is an aryl ring substituted by a group of the formula —O—(CH₂)_{q}—NR^{x}₂ or —(CH₂)ᵣ—NR^{x}₂, where q is 1-4 and r is 0-4, and each R^{x} can be H or C1-C4 alkyl, and can be substituted, or where two R^{x} can optionally cyclize into a ring. In some embodiments, this group is of the formula —O—(CH₂)_{q}-Az or —(CH₂)ᵣ-Az, where q is 1-4 and r is 0-4, and Az represents an azacyclic group such as pyrrolidine, piperidine, morpholine, piperazine, thiomorpholine, pyrrole, and the like. In some embodiments, this group is —O—(CH₂)₁₋₃-Az or —(CH₂)₁₋₃-Az, where Az is 4-morpholinyl, 1-piperazinyl, 1-pyrrolidinyl, or 1-piperidinyl; —O—CH₂—CH₂-Az or —CH₂—CH₂-Az, where Az is 4-morpholinyl is one exemplary substituent for W, when W is substituted.

Compounds of Formula I, Ia, II, IIa, IIb and IIc contain a polar substituent X, which can be at any position on the phenyl ring. In preferred embodiments, X is at position 3 or 4 of the phenyl ring. X can be any of the polar substituents described herein, but in specific embodiments, it is selected from carboxylic acid, carboxylate ester (COOR, where R is optionally substituted C1-C4 alkyl), and tetrazole, which is preferably linked to the phenyl ring via the carbon atom of the tetrazole ring.

Compounds of Formula I can, in some embodiments, have a fused ring formed by Z¹ and Z². Exemplary, non-limiting examples of the fused rings that can be formed in compounds of formula I or present in compounds of formula II, include:

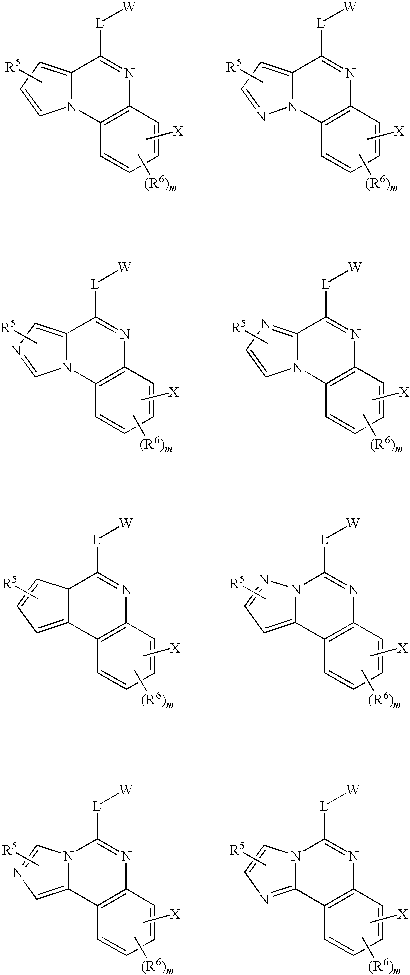

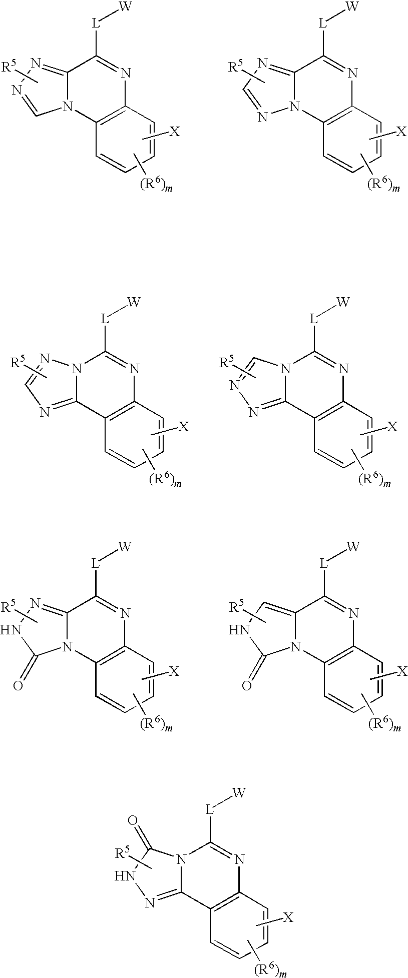

Similarly, compounds of Formula II have a fused 5-membered ring, which can contain N and/or O as ring members, and in these compounds Z¹ and Z² can both be C. The five-membered ring is aromatic, and typically at least one ring member of this ring is N or O. Exemplary fused ring systems of Formula II include:

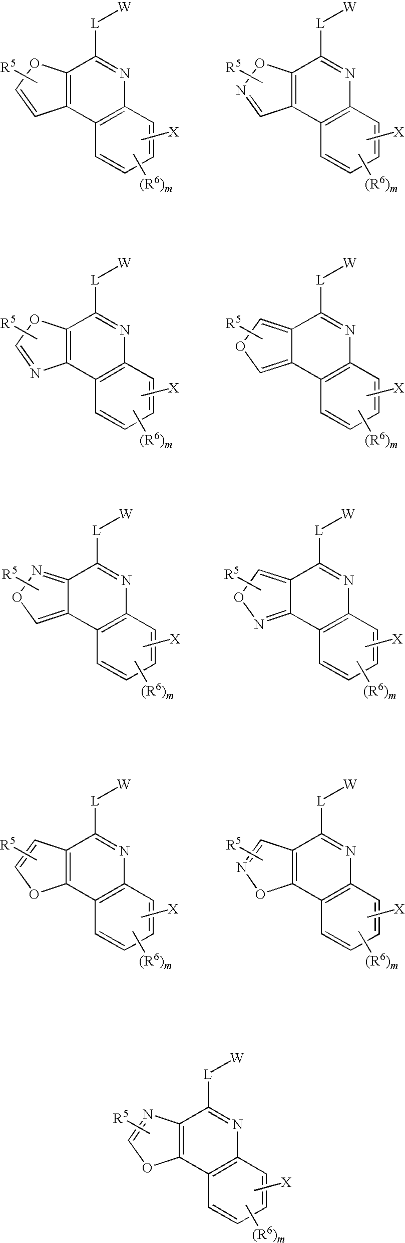

Where the above fused-ring structures have a group on the fused ring (depicted as R⁵), it can be H, or it can be Me, halo, MeO, Me₂N, COOMe, CN, CF₃, and the like, as well as any of the otherwise suitable substituents for that position.

Some specific compounds within the scope of the invention are shown in FIG. 1A-FIG. 1D. The invention includes the neutral compounds, as shown, as well as pharmaceutically acceptable salts of any one of these compounds.

Compounds of the invention can be prepared by a variety of methods that are known in the art, using reaction schemes provided in the Figures or variants thereof. For example, many compounds of Formula I or II wherein the C-ring is a pyrrole can be prepared by methods in FIG. 2, using well-known starting materials and reaction conditions.

Similar synthesis methods can be used to make compounds where the third ring (ring C) is an imidazole, as shown in FIG. 3.

A polar group is typically present on the phenyl ring of these compounds, and various polar groups can be produced by modification of the acid, ester or amide groups incorporated in the compounds by methods depicted above. For example, FIG. 4 illustrates ways to convert these acyl groups into a variety of other polar groups suitable for the compounds of the invention.

Similar interconversions can be done with compounds having other C rings, for example the compounds having an imidazole as ring C can be prepared by the same methods.

In another aspect, the invention provides pharmaceutical compositions. The pharmaceutical compositions can comprise a compound of Formula I, Ia, II, IIa, IIb or IIc as described herein, admixed with at least one pharmaceutically acceptable excipient or carrier. Preferably, the composition comprises at least two pharmaceutically acceptable excipients or carriers.

In another aspect, the invention provides a method to inhibit cell proliferation, which comprises contacting cells with a compound having a structure of Formula I, Ia, II, IIa, IIb or IIc, in an amount effective to inhibit proliferation of the cells. In certain embodiments, these cells are cells of a cancer cell line.

In particular embodiments, the cancer cell line is a breast cancer, prostate cancer, pancreatic cancer, lung cancer, hemopoietic cancer, colorectal cancer, skin cancer, or an ovarian cancer cell line. Often, the cells are in a tumor in a subject, and the compound reduces the growth rate of the tumor, or reduces the size of the tumor, or reduces the aggressiveness of the tumor, or reduces the metastasis of the tumor. In some embodiments, the compound induces apoptosis.

In certain embodiments, the methods include contacting cells, especially tumor cells, with a compound having a structure of Formula I, Ia, II, IIa, IIb or IIc, which induces apoptosis.

In certain embodiments, the cells are from an eye of a subject having macular degeneration, and the treatment method reduces the severity or symptoms or further development of macular degeneration in the subject.

In another aspect, the invention provides a method to treat a condition related to aberrant cell proliferation, which comprises administering a compound having a structure of Formula I, Ia, II, IIa, IIb or IIc to a subject in need thereof, where the compound is administered in an amount effective to treat or ameliorate the cell proliferative condition. In certain embodiments, the cell proliferative condition is a tumor-associated cancer. Specific cancers for which the compounds are useful include breast cancer, prostate cancer, pancreatic cancer, lung cancer, hematopoietic cancer, colorectal cancer, skin cancer, and ovarian cancer, colorectum, liver, lymph node, colon, prostate, brain, head and neck, skin, kidney, blood and heart.

In other embodiments, the cell proliferative condition is a non-tumor cancer. Exemplary embodiments include hematopoietic cancers, such as lymphoma and leukemia.

In other embodiments, the cell proliferative condition is macular degeneration.

In another aspect, the invention provides a method for treating pain or inflammation in a subject, which comprises administering a compound of Formula I, Ia, II, IIa, IIb or IIc to a subject in need thereof, in an amount effective to treat or reduce the pain or the inflammation.

In another aspect, the invention provides a method for inhibiting angiogenesis in a subject, which comprises administering a compound of Formula I, Ia, II, IIa, IIb or IIc to a subject in need thereof in an amount effective to inhibit the angiogenesis.

In another aspect, the invention provides a method to treat cancer, a vascular disorder, pain, inflammation, or a pathogenic infection, comprising administering to a subject in need of such treatment, an effective amount of any of the above-described compounds.

In another aspect, the invention provides a method to treat a disorder associated with excessive or aberrant CK2 or Pim kinase activity by administering to a subject in need of such treatment an effective amount of any of the above-described compounds. In some such embodiments, the disorder is selected from cancer, a vascular disorder, a pathogenic infection, and an immunological disorder.

The compounds of the invention are useful as medicaments, and are useful for the manufacture of medicaments, including medicaments to treat conditions disclosed herein, such as cancers, inflammatory conditions, infections, pain, and immunological disorders.

The terms “treat” and “treating” as used herein refer to ameliorating, alleviating, lessening, and removing symptoms of a disease or condition. A candidate molecule or compound described herein may be in a therapeutically effective amount in a formulation or medicament, which is an amount that can lead to a biological effect, such as apoptosis of certain cells (e.g., cancer cells), reduction of proliferation of certain cells, or lead to ameliorating, alleviating, lessening, or removing symptoms of a disease or condition, for example. The terms also can refer to reducing or stopping a cell proliferation rate (e.g., slowing or halting tumor growth) or reducing the number of proliferating cancer cells (e.g., removing part or all of a tumor). These terms also are applicable to reducing a titre of a microorganism in a system (i.e., cell, tissue, or subject) infected with a microorganism, reducing the rate of microbial propagation, reducing the number of symptoms or an effect of a symptom associated with the microbial infection, and/or removing detectable amounts of the microbe from the system.

Examples of microorganism include but are not limited to virus, bacterium and fungus. Thus the invention provides methods for treating protozoal disorders such as protozoan parasitosis, including infection by parasitic protozoa responsible for neurological disorders such as schizophrenia, paranoia, and encephalitis in immunocompromised patients, as well as Chagas' disease. It also provides methods to treat various viral diseases, including human immunodeficiency virus type 1 (HIV-1), human papilloma viruses (HPVs), herpes simplex virus (HSV), Epstein-Barr virus (EBV), human cytomegalovirus, hepatitis C and B viruses, influenza virus, Borna disease virus, adenovirus, coxsackievirus, coronavirus and varicella zoster virus. The methods for treating these disorders comprises administering to a subject in need thereof an effective amount of a compound of one of the formulae provided herein.

As used herein, the term “apoptosis” refers to an intrinsic cell self-destruction or suicide program. In response to a triggering stimulus, cells undergo a cascade of events including cell shrinkage, blebbing of cell membranes and chromatic condensation and fragmentation. These events culminate in cell conversion to clusters of membrane-bound particles (apoptotic bodies), which are thereafter engulfed by macrophages.

The invention in part provides pharmaceutical compositions comprising at least one compound within the scope of the invention as described herein, and methods of using compounds described herein. For example, the invention in part provides methods for identifying a candidate molecule that interacts with a CK2, Pim or Flt protein, which comprises contacting a composition containing a CK2, Pim or Flt protein and a molecule described herein with a candidate molecule and determining whether the amount of the molecule described herein that interacts with the protein is modulated, whereby a candidate molecule that modulates the amount of the molecule described herein that interacts with the protein is identified as a candidate molecule that interacts with the protein.

Provided also are methods for modulating a protein kinase activity. Protein kinases catalyze the transfer of a gamma phosphate from adenosine triphosphate to a serine or threonine amino acid (serine/threonine protein kinase), tyrosine amino acid (tyrosine protein kinase), tyrosine, serine or threonine (dual specificity protein kinase) or histidine amino acid (histidine protein kinase) in a peptide or protein substrate. Thus, included herein are methods which comprise contacting a system comprising a protein kinase protein with a compound described herein in an amount effective for modulating (e.g., inhibiting) the activity of the protein kinase. In some embodiments, the activity of the protein kinase is the catalytic activity of the protein (e.g., catalyzing the transfer of a gamma phosphate from adenosine triphosphate to a peptide or protein substrate). In certain embodiments, provided are methods for identifying a candidate molecule that interacts with a protein kinase, which comprise: contacting a composition containing a protein kinase and a compound described herein with a candidate molecule under conditions in which the compound and the protein kinase interact, and determining whether the amount of the compound that interacts with the protein kinase is modulated relative to a control interaction between the compound and the protein kinase without the candidate molecule, whereby a candidate molecule that modulates the amount of the compound interacting with the protein kinase relative to the control interaction is identified as a candidate molecule that interacts with the protein kinase. Systems in such embodiments can be a cell-free system or a system comprising cells (e.g., in vitro). The protein kinase, the compound or the molecule in some embodiments is in association with a solid phase. In certain embodiments, the interaction between the compound and the protein kinase is detected via a detectable label, where in some embodiments the protein kinase comprises a detectable label and in certain embodiments the compound comprises a detectable label. The interaction between the compound and the protein kinase sometimes is detected without a detectable label.

Provided also are compositions of matter comprising a protein kinase and a compound described herein. In some embodiments, the protein kinase in the composition is a serine-threonine protein kinase or a tyrosine protein kinase. In certain embodiments, the protein kinase is a protein kinase fragment having compound-binding activity. In some embodiments, the protein kinase in the composition is, or contains a subunit (e.g., catalytic subunit, SH2 domain, SH3 domain) of, CK2, Pim subfamily protein kinase (e.g., PIM1, PIM2, PIM3) or Flt subfamily protein kinase (e.g, FLT1, FLT3, FLT4). In certain embodiments the composition is cell free and sometimes the protein kinase is a recombinant protein.

The protein kinase can be from any source, such as cells from a mammal, ape or human, for example. Examples of serine-threonine protein kinases that can be inhibited, or may potentially be inhibited, by compounds disclosed herein include without limitation human versions of CK2, CK2α2, Pim subfamily kinases (e.g., PIM1, PIM2, PIM3), CDK1/cyclinB, c-RAF, Mer, MELK, HIPK3, HIPK2 and ZIPK. A serine-threonine protein kinase sometimes is a member of a sub-family containing one or more of the following amino acids at positions corresponding to those listed in human CK2: leucine at position 45, methionine at position 163 and isoleucine at position 174. Examples of such protein kinases include without limitation human versions of CK2, STK10, HIPK2, HIPK3, DAPK3, DYK2 and PIM-1. Examples of tyrosine protein kinases that can be inhibited, or may potentially be inhibited, by compounds disclosed herein include without limitation human versions of Flt subfamily members (e.g., FLT1, FLT2, FLT3, FLT3 (D835Y), FLT4). An example of a dual specificity protein kinase that can be inhibited, or may potentially be inhibited, by compounds disclosed herein includes without limitation DYRK2. Nucleotide and amino acid sequences for protein kinases and reagents are publicly available (e.g., World Wide Web URLs ncbi.nlm.nih.gov/sites/entrez/ and Invitrogen.com). For example, various nucleotide sequences can be accessed using the following accession numbers: NM_{—}002648.2 and NP_{—}002639.1 for PIM1; NM_{—}006875.2 and NP_{—}006866.2 for PIM2; XM_{—}938171.2 and XP_{—}943264.2 for PIM3; NM_{—}004119.2 and NP_{—}004110.2 for FLT3; NM_{—}002020.3 and NP_{—}002011.2 for FLT4; and NM_{—}002019.3 and NP_{—}002010.2 for FLT1.

The invention also in part provides methods for treating a condition related to aberrant cell proliferation. For example, provided are methods of treating a cell proliferative condition in a subject, which comprises administering a compound described herein to a subject in need thereof in an amount effective to treat the cell proliferative condition. The subject may be a research animal (e.g., rodent, dog, cat, monkey), optionally containing a tumor such as a xenograft tumor (e.g., human tumor), for example, or may be a human. A cell proliferative condition sometimes is a tumor or non-tumor cancer, including but not limited to, cancers of the colorectum, breast, lung, liver, pancreas, lymph node, colon, prostate, brain, head and neck, skin, liver, kidney, blood and heart (e.g., leukemia, lymphoma, carcinoma).

Compounds and compositions of the invention may be used alone or in combination with anticancer or other agents, such as a palliative agents, that are typically administered to a patient being treated for cancer, as further described herein.

Also provided are methods for treating a condition related to inflammation or pain. For example, provided are methods of treating pain in a subject, which comprise administering a compound described herein to a subject in need thereof in an amount effective to treat the pain. Provided also are methods of treating inflammation in a subject, which comprises administering a compound described herein to a subject in need thereof in an amount effective to treat the inflammation. The subject may be a research animal (e.g., rodent, dog, cat, monkey), for example, or may be a human. Conditions associated with inflammation and pain include without limitation acid reflux, heartburn, acne, allergies and sensitivities, Alzheimer's disease, asthma, atherosclerosis, bronchitis, carditis, celiac disease, chronic pain, Crohn's disease, cirrhosis, colitis, dementia, dermatitis, diabetes, dry eyes, edema, emphysema, eczema, fibromyalgia, gastroenteritis, gingivitis, heart disease, hepatitis, high blood pressure, insulin resistance, interstitial cystitis, joint pain/arthritis/rheumatoid arthritis, metabolic syndrome (syndrome X), myositis, nephritis, obesity, osteopenia, glomerulonephritis (GN), juvenile cystic kidney disease, and type I nephronophthisis (NPHP), osteoporosis, Parkinson's disease, Guam-Parkinson dementia, supranuclear palsy, Kuf's disease, and Pick's disease, as well as memory impairment, brain ischemia, and schizophrenia, periodontal disease, polyarteritis, polychondritis, psoriasis, scleroderma, sinusitis, Sjögren's syndrome, spastic colon, systemic candidiasis, tendonitis, urinary track infections, vaginitis, inflammatory cancer (e.g., inflammatory breast cancer) and the like. Methods for determining effects of compounds herein on pain or inflammation are known. For example, formalin-stimulated pain behaviors in research animals can be monitored after administration of a compound described herein to assess treatment of pain (e.g., Li et al., Pain 115(1-2): 182-90 (2005)). Also, modulation of pro-inflammatory molecules (e.g., IL-8, GRO-alpha, MCP-1, TNFalpha and iNOS) can be monitored after administration of a compound described herein to assess treatment of inflammation (e.g., Parhar et al., Int J Colorectal Dis. 22(6): 601-9 (2006)), for example. Thus, also provided are methods for determining whether a compound herein reduces inflammation or pain, which comprise contacting a system with a compound described herein in an amount effective for modulating (e.g., inhibiting) the activity of a pain signal or inflammation signal. Provided also are methods for identifying a compound that reduces inflammation or pain, which comprise: contacting a system with a compound of Formula I, II, Ia, or IIa; and detecting a pain signal or inflammation signal, whereby a compound that modulates the pain signal relative to a control molecule is identified as a compound that reduces inflammation of pain. Non-limiting examples of pain signals are formalin-stimulated pain behaviors and examples of inflammation signals include without limitation a level of a pro-inflammatory molecule. The invention thus in part pertains to methods for modulating angiogenesis in a subject, and methods for treating a condition associated with aberrant angiogenesis in a subject. proliferative diabetic retinopathy.

CK2 has also been shown to play a role in the pathogenesis of atherosclerosis, and may prevent atherogenesis by maintaining laminar shear stress flow. CK2 plays a role in vascularization, and has been shown to mediate the hypoxia-induced activation of histone deacetylases (HDACs). CK2 is also involved in diseases relating to skeletal muscle and bone tissue, including, e.g., cardiomyocyte hypertrophy, heart failure, impaired insulin signaling and insulin resistance, hypophosphatemia and inadequate bone matrix mineralization.

Thus in one aspect, the invention provides methods to treat these conditions, comprising administering to a subject in need of such treatment an effect amount of a CK2 inhibitor, such as a compound of Formula I or II.

Also provided are methods for treating an angiogenesis condition, which comprise administering a compound described herein to a subject in need thereof, in an amount effective to treat the angiogenesis condition. Angiogenesis conditions include without limitation solid tumor cancers, varicose disease, and the like.

Also provided are methods for treating a condition associated with an aberrant immune response in a subject, which comprise administering a compound described herein to a subject in need thereof, in an amount effective to treat the condition. Conditions characterized by an aberrant immune response include without limitation, organ transplant rejection, asthma, autoimmune disorders, including rheumatoid arthritis, multiple sclerosis, myasthenia gravis, systemic lupus erythematosus, scleroderma, polymyositis, mixed connective tissue disease (MCTD), Crohn's disease, and ulcerative colitis. In certain embodiments, an immune response may be modulated by administering a compound herein in combination with a molecule that modulates (e.g., inhibits) the biological activity of an mTOR pathway member or member of a related pathway (e.g., mTOR, PI3 kinase, AKT). In certain embodiments the molecule that modulates the biological activity of an mTOR pathway member or member of a related pathway is rapamycin. In certain embodiments, provided herein is a composition comprising a compound described herein in combination with a molecule that modulates the biological activity of an mTOR pathway member or member of a related pathway, such as rapamycin, for example.

In preferred embodiments of the present invention, the compound is a compound of Formula I, Ia, II or IIa in one of the lists of compounds provided herein, or a pharmaceutically acceptable salt of one of these compounds.

### Formulations and Routes of Administration

Any suitable formulation of a compound described above can be prepared for administration. Any suitable route of administration may be used, including, but not limited to, oral, parenteral, intravenous, intramuscular, transdermal, topical and subcutaneous routes. Depending on the subject to be treated, the mode of administration, and the type of treatment desired—e.g., prevention, prophylaxis, therapy; the compounds are formulated in ways consonant with these parameters. Preparation of suitable formulations for each route of administration are known in the art. A summary of such formulation methods and techniques is found in Remington's Pharmaceutical Sciences, latest edition, Mack Publishing Co., Easton, Pa., which is incorporated herein by reference. The formulation of each substance or of the combination of two substances will generally include a diluent as well as, in some cases, adjuvants, buffers, preservatives and the like. The substances to be administered can be administered also in liposomal compositions or as microemulsions.

For injection, formulations can be prepared in conventional forms as liquid solutions or suspensions or as solid forms suitable for solution or suspension in liquid prior to injection or as emulsions. Suitable excipients include, for example, water, saline, dextrose, glycerol and the like. Such compositions may also contain amounts of nontoxic auxiliary substances such as wetting or emulsifying agents, pH buffering agents and the like, such as, for example, sodium acetate, sorbitan monolaurate, and so forth.

Various sustained release systems for drugs have also been devised, and can be applied to compounds of the invention. See, for example, U.S. Pat. No. 5,624,677, the methods of which are incorporated herein by reference.

Systemic administration may also include relatively noninvasive methods such as the use of suppositories, transdermal patches, transmucosal delivery and intranasal administration. Oral administration is also suitable for compounds of the invention. Suitable forms include syrups, capsules, tablets, as is understood in the art.

The compounds of the invention, or their pharmaceutically acceptable salts, are administered in a therapeutically effective amount which will vary depending upon a variety of factors including the activity of the specific compound employed, the metabolic stability and length of action of the compound, the age, body weight, general health, sex, diet, mode and time of administration, rate of excretion, drug combination, the severity of the particular disease-states, and the host undergoing therapy. The compounds of the present invention can be administered to a patient at dosage levels in the range of about 0.1 to about 1,000 mg per day. For a normal human adult having a body weight of about 70 kilograms, a dosage in the range of about 0.01 to about 100 mg per kilogram of body weight per day is an example. The specific dosage used, however, can vary. For example, the dosage can depend on a number of factors including the requirements of the patient, the severity of the condition being treated, and the pharmacological activity of the compound being used. The determination of optimum dosages for a particular patient is well known to one of ordinary skill in the art.

For administration to animal or human subjects, the appropriate dosage of a compound described above often is 0.01-15 mg/kg, and sometimes 0.1-10 mg/kg. Dosage levels are dependent on the nature of the condition, drug efficacy, the condition of the patient, the judgment of the practitioner, and the frequency and mode of administration; however, optimization of such parameters is within the ordinary level of skill in the art.

### Therapeutic Combinations

Compounds of the invention may be used alone or in combination with another therapeutic agent. The invention provides methods to treat conditions such as cancer, inflammation and immune disorders by administering to a subject in need of such treatment a therapeutically effective amount of a therapeutic agent useful for treating said disorder and administering to the same subject a therapeutically effective amount of a modulator of the present invention. A CK2, Pim or Flt modulator is an agent that inhibits or enhances a biological activity of a CK2 protein, a Pim protein or a Flt protein, and is generically referred to hereafter as a “modulator.” The therapeutic agent and the modulator may be administered together, either as separate pharmaceutical compositions or admixed in a single pharmaceutical composition. The therapeutic agent and the modulator may also be administered separately, including at different times and with different frequencies. The modulator may be administered by any known route, such as orally, intravenously, intramuscularly, nasally, and the like; and the therapeutic agent may also be administered by any conventional route. In many embodiments, at least one and optionally both of the modulator and the therapeutic agent may be administered orally.

In certain embodiments, a “modulator” as described above may be used in combination with a therapeutic agent that can act by binding to regions of DNA that can form certain quadruplex structures. In such embodiments, the therapeutic agents have anticancer activity on their own, but their activity is enhanced when they are used in combination with a modulator. This synergistic effect allows the therapeutic agent to be administered in a lower dosage while achieving equivalent or higher levels of at least one desired effect.

A modulator may be separately active for treating a cancer. For combination therapies described above, when used in combination with a therapeutic agent, the dosage of a modulator will frequently be two-fold to ten-fold lower than the dosage required when the modulator is used alone to treat the same condition or subject. Determination of a suitable amount of the modulator for use in combination with a therapeutic agent is readily determined by methods known in the art.

Compounds and compositions of the invention may be used in combination with anticancer or other agents, such as palliative agents, that are typically administered to a patient being treated for cancer. Such “anticancer agents” include, e.g., classic chemotherapeutic agents, as well as molecular targeted therapeutic agents, biologic therapy agents, and radiotherapeutic agents.

When a compound or composition of the invention is used in combination with an anticancer agent to another agent, the present invention provides, for example, simultaneous, staggered, or alternating treatment. Thus, the compound of the invention may be administered at the same time as an anticancer agent, in the same pharmaceutical composition; the compound of the invention may be administered at the same time as the anticancer agent, in separate pharmaceutical compositions; the compound of the invention may be administered before the anticancer agent, or the anticancer agent may be administered before the compound of the invention, for example, with a time difference of seconds, minutes, hours, days, or weeks.

In examples of a staggered treatment, a course of therapy with the compound of the invention may be administered, followed by a course of therapy with the anticancer agent, or the reverse order of treatment may be used, and more than one series of treatments with each component may also be used. In certain examples of the present invention, one component, for example, the compound of the invention or the anticancer agent, is administered to a mammal while the other component, or its derivative products, remains in the bloodstream of the mammal. For example, a compound of formula I, Ia, II or IIa may be administered while the anticancer agent or its derivative products remains in the bloodstream, or the anticancer agent may be administered while the compound of formulae I, Ia, II or IIa or its derivatives remains in the bloodstream. In other examples, the second component is administered after all, or most of the first component, or its derivatives, have left the bloodstream of the mammal.

The compound of the invention and the anticancer agent may be administered in the same dosage form, e.g., both administered as intravenous solutions, or they may be administered in different dosage forms, e.g., one compound may be administered topically and the other orally. A person of ordinary skill in the art would be able to discern which combinations of agents would be useful based on the particular characteristics of the drugs and the cancer involved.

Anticancer agents useful in combination with the compounds of the present invention may include agents selected from any of the classes known to those of ordinary skill in the art, including, but not limited to, antimicrotubule agents such as diterpenoids and vinca alkaloids; platinum coordination complexes; alkylating agents such as nitrogen mustards, oxazaphosphorines, alkylsulfonates, nitrosoureas, and triazenes; antibiotic agents such as anthracyclins, actinomycins and bleomycins; topoisomerase II inhibitors such as epipodophyllotoxins; antimetabolites such as purine and pyrimidine analogues and anti-folate compounds; topoisomerase I inhibitors such as camptothecins; hormones and hormonal analogues; signal transduction pathway inhibitors; nonreceptor tyrosine kinase angiogenesis inhibitors; immunotherapeutic agents; pro-apoptotic agents; and cell cycle signaling inhibitors; other agents.

Anti-microtubule or anti-mitotic agents are phase specific agents that are typically active against the microtubules of tumor cells during M or the mitosis phase of the cell cycle. Examples of anti-microtubule agents include, but are not limited to, diterpenoids and vinca alkaloids.

Diterpenoids, which are derived from natural sources, are phase specific anti-cancer agents that are believed to operate at the G2/M phases of the cell cycle. It is believed that the diterpenoids stabilize the p-tubulin subunit of the microtubules, by binding with this protein. Disassembly of the protein appears then to be inhibited with mitosis being arrested and cell death following.

Examples of diterpenoids include, but are not limited to, taxanes such as paclitaxel, docetaxel, larotaxel, ortataxel, and tesetaxel. Paclitaxel is a natural diterpene product isolated from the Pacific yew tree Taxus brevifolia and is commercially available as an injectable solution TAXOL®. Docetaxel is a semisynthetic derivative of paclitaxel q. v., prepared using a natural precursor, 10-deacetyl-baccatin III, extracted from the needle of the European Yew tree. Docetaxel is commercially available as an injectable solution as TAXOTERE®.

Vinca alkaloids are phase specific anti-neoplastic agents derived from the periwinkle plant. Vinca alkaloids that are believed to act at the M phase (mitosis) of the cell cycle by binding specifically to tubulin. Consequently, the bound tubulin molecule is unable to polymerize into microtubules. Mitosis is believed to be arrested in metaphase with cell death following. Examples of vinca alkaloids include, but are not limited to, vinblastine, vincristine, vindesine, and vinorelbine. Vinblastine, vincaleukoblastine sulfate, is commercially available as VELBAN® as an injectable solution. Vincristine, vincaleukoblastine 22-oxo-sulfate, is commercially available as ONCOVIN® as an injectable solution. Vinorelbine, is commercially available as an injectable solution of vinorelbine tartrate (NAVELBINE®), and is a semisynthetic vinca alkaloid derivative.

Platinum coordination complexes are non-phase specific anti-cancer agents, which are interactive with DNA. The platinum complexes are believed to enter tumor cells, undergo, aquation and form intra- and interstrand crosslinks with DNA causing adverse biological effects to the tumor. Platinum-based coordination complexes include, but are not limited to cisplatin, carboplatin, nedaplatin, oxaliplatin, satraplatin, and (SP-4-3)-(cis)-amminedichloro-[2-methylpyridine]platinum(II). Cisplatin, cis-diamminedichloroplatinum, is commercially available as PLATINOL® as an injectable solution. Carboplatin, platinum, diammine [1,1-cyclobutane-dicarboxylate(2-)-0,0′] is commercially available as PARAPLATIN® as an injectable solution.

Alkylating agents are generally non-phase specific agents and typically are strong electrophiles. Typically, alkylating agents form covalent linkages, by alkylation, to DNA through nucleophilic moieties of the DNA molecule such as phosphate, amino, sulfhydryl, hydroxyl, carboxyl, and imidazole groups. Such alkylation disrupts nucleic acid function leading to cell death. Examples of alkylating agents include, but are not limited to, alkyl sulfonates such as busulfan; ethyleneimine and methylmelamine derivatives such as altretamine and thiotepa; nitrogen mustards such as chlorambucil, cyclophosphamide, estramustine, ifosfamide, mechlorethamine, melphalan, and uramustine; nitrosoureas such as carmustine, lomustine, and streptozocin; triazenes and imidazotetrazines such as dacarbazine, procarbazine, temozolamide, and temozolomide. Cyclophosphamide, 2-[bis(2-chloroethyl)-amino]tetrahydro-2H-1,3,2-oxazaphosphorine 2-oxide monohydrate, is commercially available as an injectable solution or tablets as CYTOXAN®. Melphalan, 4-[bis(2-chloroethyl)amino]-L-phenylalanine, is commercially available as an injectable solution or tablets as ALKERAN®. Chlorambucil, 4-[bis(2-chloroethyl)amino]-benzenebutanoic acid, is commercially available as LEUKERAN® tablets. Busulfan, 1,4-butanediol dimethanesulfonate, is commercially available as MYLERAN® TABLETS. Carmustine, 1,3-[bis(2-chloroethyl)-1-nitrosourea, is commercially available as single vials of lyophilized material as BiCNU®. 5-(3,3-dimethyl-1-triazeno)-imidazole-4-carboxamide, is commercially available as single vials of material as DTIC-Dome®.

Anti-tumor antibiotics are non-phase specific agents which are believed to bind or intercalate with DNA. This may result in stable DNA complexes or strand breakage, which disrupts ordinary function of the nucleic acids, leading to cell death. Examples of anti-tumor antibiotic agents include, but are not limited to, anthracyclines such as daunorubicin (including liposomal daunorubicin), doxorubicin (including liposomal doxorubicin), epirubicin, idarubicin, and valrubicin; streptomyces-related agents such as bleomycin, actinomycin, mithramycin, mitomycin, porfiromycin; and mitoxantrone. Dactinomycin, also know as Actinomycin D, is commercially available in injectable form as COSMEGEN®. Daunorubicin, (8S-cis-)-8-acetyl-10-[(3-amino-2,3,6-trideoxy-a-L-lyxohexopyranosyl)oxy]-7,8,9,10-tetrahydro-6,8,11-trihydroxy-1-methoxy-5,12-naphthacenedione hydrochloride, is commercially available as a liposomal injectable form as DAUNOXOME® or as an injectable as CERUBIDINE®. Doxorubicin, (8S, 10S)-10-[(3-amino-2,3,6-trideoxy-α-L-lyxohexopyranosyl)oxy]-8-glycoloyl, 7,8,9,10-tetrahydro-6,8,11-trihydroxy-1-methoxy-5,12-naphthacenedione hydrochloride, is commercially available in an injectable form as RUBEX® or ADRIAMYCIN RDF®. Bleomycin, a mixture of cytotoxic glycopeptide antibiotics isolated from a strain of Streptomyces verticil/us, is commercially available as BLENOXANE®.

Topoisomerase II inhibitors include, but are not limited to, epipodophyllotoxins, which are phase specific anti-neoplastic agents derived from the mandrake plant. Epipodophyllotoxins typically affect cells in the S and G2 phases of the cell cycle by forming a ternary complex with topoisomerase II and DNA causing DNA strand breaks. The strand breaks accumulate and cell death follows. Examples of epipodophyllotoxins include, but are not limited to, etoposide, teniposide, and amsacrine. Etoposide, 4′-demethyl-epipodophyllotoxin 9[4,6-0-(R)-ethylidene-β-D-glucopyranoside], is commercially available as an injectable solution or capsules as VePESID® and is commonly known as VP-16. Teniposide, 4′-demethyl-epipodophyllotoxin 9[4,6-0-(R)-thenylidene-β-D-glucopyranoside], is commercially available as an injectable solution as VUMON® and is commonly known as VM-26.

Antimetabolite neoplastic agents are phase specific anti-neoplastic agents that typically act at S phase (DNA synthesis) of the cell cycle by inhibiting DNA synthesis or by inhibiting purine or pyrimidine base synthesis and thereby limiting DNA synthesis. Consequently, S phase does not proceed and cell death follows. Anti-metabolites, include purine analogs, such as fludarabine, cladribine, chlorodeoxyadenosine, clofarabine, mercaptopurine, pentostatin, erythrohydroxynonyladenine, fludarabine phosphate and thioguanine; pyrimidine analogs such as fluorouracil, gemcitabine, capecitabine, cytarabine, azacitidine, edatrexate, floxuridine, and troxacitabine; antifolates, such as methotrexate, pemetrexed, raltitrexed, and trimetrexate. Cytarabine, 4-amino-1-p-D-arabinofuranosyl-2 (1H)-pyrimidinone, is commercially available as CYTOSAR-U® and is commonly known as Ara-C. Mercaptopurine, 1,7-dihydro-6H-purine-6-thione monohydrate, is commercially available as PURINETHOL®. Thioguanine, 2-amino-1,7-dihydro-6H-purine-6-thione, is commercially available as TABLOID®. Gemcitabine, 2′-deoxy-2′,2′-difluorocytidine monohydrochloride (p-isomer), is commercially available as GEMZAR®.

Topoisomerase I inhibitors including, camptothecin and camptothecin derivatives. Examples of topoisomerase I inhibitors include, but are not limited to camptothecin, topotecan, irinotecan, rubitecan, belotecan and the various optical forms (i.e., (R), (S) or (R,S)) of 7-(4-methylpiperazino-methylene)-10,11-ethylenedioxy-camptothecin, as described in U.S. Pat. Nos. 6,063,923; 5,342,947; 5,559,235; 5,491,237 and pending U.S. patent application Ser. No. 08/977,217 filed Nov. 24, 1997. Irinotecan HCl, (4S)-4,11-diethyl-4-hydroxy-9-[(4-piperidinopiperidino)-carbonyloxy]-1H-pyrano[3′,4′,6,7]indolizino[1,2-b]quinoline-3,14(4H, 12H)-dione hydrochloride, is commercially available as the injectable solution CAMPT0SAR®. Irinotecan is a derivative of camptothecin which binds, along with its active metabolite 8N-38, to the topoisomerase I-DNA complex. Topotecan HCl, (S)-10-[(dimethylamino)methyl]-4-ethyl-4,9-dihydroxy-1H-pyrano[3′,4′,6,7]indolizino[1,2-b]quinoline-3,14-(4H, 12H)-dione monohydrochloride, is commercially available as the injectable solution HYCAMTIN®.

Hormones and hormonal analogues are useful compounds for treating cancers in which there is a relationship between the hormone(s) and growth and/or lack of growth of the cancer. Examples of hormones and hormonal analogues useful in cancer treatment include, but are not limited to, androgens such as fluoxymesterone and testolactone; antiandrogens such as bicalutamide, cyproterone, flutamide, and nilutamide; aromatase inhibitors such as aminoglutethimide, anastrozole, exemestane, formestane, vorazole, and letrozole; corticosteroids such as dexamethasone, prednisone and prednisolone; estrogens such as diethylstilbestrol; antiestrogens such as fulvestrant, raloxifene, tamoxifen, toremifene, droloxifene, and iodoxyfene, as well as selective estrogen receptor modulators (SERMS) such those described in U.S. Pat. Nos. 5,681,835, 5,877,219, and 6,207,716; 5α-reductases such as finasteride and dutasteride; gonadotropin-releasing hormone (GnRH) and analogues thereof which stimulate the release of leutinizing hormone (LH) and/or follicle stimulating hormone (FSH), for example LHRH agonists and antagonists such as buserelin, goserelin, leuprolide, and triptorelin; progestins such as medroxyprogesterone acetate and megestrol acetate; and thyroid hormones such as levothyroxine and liothyronine.

Signal transduction pathway inhibitors are those inhibitors, which block or inhibit a chemical process which evokes an intracellular change, such as cell proliferation or differentiation. Signal tranduction inhibitors useful in the present invention include, e.g., inhibitors of receptor tyrosine kinases, non-receptor tyrosine kinases, SH2/SH3 domain blockers, serine/threonine kinases, phosphotidyl inositol-3 kinases, myo-inositol signaling, and Ras oncogenes.

Several protein tyrosine kinases catalyse the phosphorylation of specific tyrosyl residues in various proteins involved in the regulation of cell growth. Such protein tyrosine kinases can be broadly classified as receptor or non-receptor kinases. Receptor tyrosine kinases are transmembrane proteins having an extracellular ligand binding domain, a transmembrane domain, and a tyrosine kinase domain. Receptor tyrosine kinases are involved in the regulation of cell growth and are sometimes termed growth factor receptors.

Inappropriate or uncontrolled activation of many of these kinases, for example by over-expression or mutation, has been shown to result in uncontrolled cell growth. Accordingly, the aberrant activity of such kinases has been linked to malignant tissue growth. Consequently, inhibitors of such kinases could provide cancer treatment methods.

Growth factor receptors include, for example, epidermal growth factor receptor (EGFr), platelet derived growth factor receptor (PDGFr), erbB2, erbB4, vascular endothelial growth factor receptor (VEGFr), tyrosine kinase with immunoglobulin-like and epidermal growth factor homology domains (TIE-2), insulin growth factor-I (IGFI) receptor, macrophage colony stimulating factor (cfms), BTK, ckit, cmet, fibroblast growth factor (FGF) receptors, Trk receptors (TrkA, TrkB, and TrkC), ephrin (eph) receptors, and the RET protooncogene.

Several inhibitors of growth receptors are under development and include ligand antagonists, antibodies, tyrosine kinase inhibitors and anti-sense oligonucleotides. Growth factor receptors and agents that inhibit growth factor receptor function are described, for instance, in Kath, John C., Exp. Opin. Ther. Patents (2000) 10(6):803-818; Shawver et al., Drug Discov. Today (1997), 2(2):50-63; and Lofts, F. J. et al., “Growth factor receptors as targets”, New Molecular Targets for Cancer Chemotherapy, ed. Workman, Paul and Kerr, David, CRC press 1994, London. Specific examples of receptor tyrosine kinase inhibitors include, but are not limited to, sunitinib, erlotinib, gefitinib, and imatinib.

Tyrosine kinases which are not growth factor receptor kinases are termed non-receptor tyrosine kinases. Non-receptor tyrosine kinases useful in the present invention, which are targets or potential targets of anti-cancer drugs, include cSrc, Lck, Fyn, Yes, Jak, cAbl, FAK (Focal adhesion kinase), Brutons tyrosine kinase, and Bcr-Abl. Such non-receptor kinases and agents which inhibit non-receptor tyrosine kinase function are described in Sinh, S. and Corey, S. J., J. Hematotherapy & Stem Cell Res. (1999) 8(5): 465-80; and Bolen, J. B., Brugge, J. S., Annual Review of Immunology. (1997) 15: 371-404.

SH2/SH3 domain blockers are agents that disrupt SH2 or SH3 domain binding in a variety of enzymes or adaptor proteins including, PI3-K p85 subunit, Src family kinases, adaptor molecules (Shc, Crk, Nck, Grb2) and Ras-GAP. SH2/SH3 domains as targets for anti-cancer drugs are discussed in Smithgall, T. E., J. Pharmacol. Toxicol. Methods. (1995), 34(3): 125-32 Inhibitors of Serine/Threonine Kinases including MAP kinase cascade blockers which include blockers of Raf kinases (rafk), Mitogen or Extracellular Regulated Kinase (MEKs), and Extracellular Regulated Kinases (ERKs); and Protein kinase C family member blockers including blockers of PKCs (alpha, beta, gamma, epsilon, mu, lambda, iota, zeta). IkB kinase family (IKKa, IKKb), PKB family kinases, AKT kinase family members, and TGF beta receptor kinases. Such Serine/Threonine kinases and inhibitors thereof are described in Yamamoto, T., Taya, S., Kaibuchi, K., J. Biochemistry. (1999) 126 (5): 799-803; Brodt, P, Samani, A, & Navab, R, Biochem. Pharmacol. (2000) 60:1101-1107; Massague, J., Weis-Garcia, F., Cancer Surv. (1996) 27:41-64; Philip, P. A, and Harris, A L, Cancer Treat. Res. (1995) 78: 3-27; Lackey, K. et al. Bioorg. Med. Chem. Letters, (2000) 10(3): 223-226; U.S. Pat. No. 6,268,391; and Martinez-Lacaci, I., et al., Int. J. Cancer (2000), 88(1): 44-52. Inhibitors of Phosphotidyl inositol-3 Kinase family members including blockers of PI3-kinase, ATM, DNA-PK, and Ku are also useful in the present invention. Such kinases are discussed in Abraham, R T. Current Opin. Immunol. (1996), 8(3): 412-8; Canman, C. E., Lim, D. S., Oncogene (1998) 17(25): 3301-8; Jackson, S. P., Int. J. Biochem. Cell Biol. (1997) 29(7):935-8; and Thong, H. et al., Cancer Res. (2000) 60(6):1541-5. Also useful in the present invention are Myo-inositol signaling inhibitors such as phospholipase C blockers and Myoinositol analogues. Such signal inhibitors are described in Powis, G., and Kozikowski A, (1994) New Molecular Targets for Cancer Chemotherapy, ed., Paul Workman and David Kerr, CRC Press 1994, London.

Another group of signal transduction pathway inhibitors are inhibitors of Ras Oncogene. Such inhibitors include inhibitors of farnesyltransferase, geranyl-geranyl transferase, and CAAX proteases as well as anti-sense oligonucleotides, ribozymes and immunotherapy. Such inhibitors have been shown to block ras activation in cells containing wild type mutant ras, thereby acting as antiproliferation agents. Ras oncogene inhibition is discussed in Scharovsky, O. G., Rozados, V. R, Gervasoni, S I, Matar, P., J. Biomed. Sci. (2000) 7(4): 292-8; Ashby, M. N., Curr. Opin. Lipidol. (1998) 9(2): 99-102; and Oliff, A., Biochim. Biophys. Acta, (1999) 1423(3):C19-30.

As mentioned above, antibody antagonists to receptor kinase ligand binding may also serve as signal transduction inhibitors. This group of signal transduction pathway inhibitors includes the use of humanized antibodies to the extracellular ligand binding domain of receptor tyrosine kinases. For example Imclone C225 EGFR specific antibody (see Green, M. C. et al., Cancer Treat. Rev., (2000) 26(4): 269-286); Herceptin® erbB2 antibody (see Stern, D F, Breast Cancer Res. (2000) 2(3):176-183); and 2CB VEGFR2 specific antibody (see Brekken, R. A. et al., Cancer Res. (2000) 60(18):5117-24).

Non-receptor kinase angiogenesis inhibitors may also find use in the present invention Inhibitors of angiogenesis related VEGFR and TIE2 are discussed above in regard to signal transduction inhibitors (both receptors are receptor tyrosine kinases). Angiogenesis in general is linked to erbB2/EGFR signaling since inhibitors of erbB2 and EGFR have been shown to inhibit angiogenesis, primarily VEGF expression. Thus, the combination of an erbB2/EGFR inhibitor with an inhibitor of angiogenesis makes sense. Accordingly, non-receptor tyrosine kinase inhibitors may be used in combination with the EGFR/erbB2 inhibitors of the present invention. For example, anti-VEGF antibodies, which do not recognize VEGFR (the receptor tyrosine kinase), but bind to the ligand; small molecule inhibitors of integrin (alphav beta3) that will inhibit angiogenesis; endostatin and angiostatin (non-RTK) may also prove useful in combination with the disclosed erb family inhibitors. (See Bruns, C J et al., Cancer Res. (2000), 60(11): 2926-2935; Schreiber A B, Winkler M E, & Derynck R., Science (1986) 232(4755):1250-53; Yen L. et al., Oncogene (2000) 19(31): 3460-9).

Agents used in immunotherapeutic regimens may also be useful in combination with the compounds of formula (I)-(II). There are a number of immunologic strategies to generate an immune response against erbB2 or EGFR. These strategies are generally in the realm of tumor vaccinations. The efficacy of immunologic approaches may be greatly enhanced through combined inhibition of erbB2/EGFR signaling pathways using a small molecule inhibitor. Discussion of the immunologic/tumor vaccine approach against erbB2/EGFR are found in Reilly R T, et al., Cancer Res. (2000) 60(13):3569-76; and Chen Y, et al., Cancer Res. (1998) 58(9):1965-71.

Agents used in pro-apoptotic regimens (e.g., bcl-2 antisense oligonucleotides) may also be used in the combination of the present invention. Members of the Bcl-2 family of proteins block apoptosis. Upregulation of bcl-2 has therefore been linked to chemoresistance. Studies have shown that the epidermal growth factor (EGF) stimulates anti-apoptotic members of the bcl-2 family. Therefore, strategies designed to downregulate the expression of bcl-2 in tumors have demonstrated clinical benefit and are now in Phase II/III trials, namely Genta's G3139 bcl-2 antisense oligonucleotide. Such pro-apoptotic strategies using the antisense oligonucleotide strategy for bcl-2 are discussed in Waters J S, et al., J. Clin. Oncol. (2000) 18(9): 1812-23; and Kitada S, et al. Antisense Res. Dev. (1994) 4(2): 71-9.

Cell cycle signalling inhibitors inhibit molecules involved in the control of the cell cycle. A family of protein kinases called cyclin dependent kinases (CDKs) and their interaction with a family of proteins termed cyclins controls progression through the eukaryotic cell cycle. The coordinate activation and inactivation of different cyclin/CDK complexes is necessary for normal progression through the cell cycle. Several inhibitors of cell cycle signalling are under development. For instance, examples of cyclin dependent kinases, including CDK2, CDK4, and CDK6 and inhibitors for the same are described in, for instance, Rosania G R & Chang Y-T., Exp. Opin. Ther. Patents (2000) 10(2):215-30.

Other molecular targeted agents include FKBP binding agents, such as the immunosuppressive macrolide antibiotic, rapamycin; gene therapy agents, antisense therapy agents, and gene expression modulators such as the retinoids and rexinoids, e.g. adapalene, bexarotene, trans-retinoic acid, 9-cisretinoic acid, and N-(4 hydroxyphenyl)retinamide; phenotype-directed therapy agents, including: monoclonal antibodies such as alemtuzumab, bevacizumab, cetuximab, ibritumomab tiuxetan, rituximab, and trastuzumab; immunotoxins such as gemtuzumab ozogamicin, radioimmunoconjugates such as 131-tositumomab; and cancer vaccines.

Miscellaneous agents include altretamine, arsenic trioxide, gallium nitrate, hydroxyurea, levamisole, mitotane, octreotide, procarbazine, suramin, thalidomide, photodynamic compounds such as methoxsalen and sodium porfimer, and proteasome inhibitors such as bortezomib.

Biologic therapy agents include: interferons such as interferon-u2a and interferon-u2b, and interleukins such as aldesleukin, denileukin diftitox, and oprelvekin.

In addition to these anticancer agents intended to act against cancer cells, combination therapies including the use of protective or adjunctive agents, including: cytoprotective agents such as armifostine, dexrazonxane, and mesna, phosphonates such as pamidronate and zoledronic acid, and stimulating factors such as epoetin, darbeopetin, filgrastim, PEG-filgrastim, and sargramostim, are also envisioned.

### Processes for Synthesizing Compounds of Formula I and II

Compounds of the invention can be prepared using available methods and reagents, based on the ordinary level of skill in the art, for example, using the processes described in FIGS. 2-12 or modifications thereof, and in the examples provided below.

For example, many of the compounds of Formula I can be prepared according to the reactions in FIG. 5, which describes a general synthesis of quinolone intermediates where the C-ring is a pyrrole.

Compounds wherein the C-ring is an imidazole can be prepared according to the scheme in FIG. 6, while compounds having a triazole as the C-ring can be made by similar methods, as illustrated in FIG. 7.

Similarly, compounds wherein the C-ring is a triazolone can be made by the reaction scheme illustrated in FIG. 8.

Compounds wherein the C-ring is a pyrazole can be made by the reaction scheme illustrated in FIG. 9.

Triazoloquinoline compounds, where the C-ring is a 1,2,4 triazine, can be made by reaction scheme illustrated in FIG. 10. Other triazine-containing derivatives can be made as shown here, using chemistry from a U.S. Pat. No. 6,207,693 to construct the bicyclic quinazoline core, as shown in FIG. 11.

Compounds lacking a C-ring can also be made, e.g., by following exemplary reaction scheme shown in FIG. 12.

The following examples illustrate and do not limit the invention.

### Example 1

### Processes for Synthesizing Quinoxalinone Intermediates

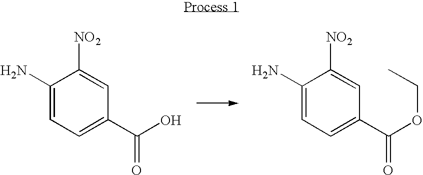

4-amino-3-nitrobenzoic acid (3.64 g) was heated at reflux overnight in Ethanol (100 ml) and sulfuric acid (2 ml). LCMS showed completion of the reaction. The mixture was cooled to room temperature and concentrated. The mixture was partitioned between diethylether and water. The organic phase was dried over Na₂SO₄ and the solvent removed in vacuo to afford ethyl 4-amino-3-nitrobenzoate (4.05 g, 96% yield). LCMS (ES): >85% pure, m/z 211 [M+1]⁺.

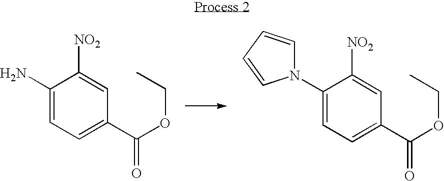

ethyl 4-amino-3-nitrobenzoate (1.0 eq., 3.05 g, 14.5 mmol) was combined with 2,5-dimethoxytetrahydrofuran (1.1 eq, 2.10 g, 16.0 mmol) in acetic acid (10 ml) and the mixture was refluxed for 1 hours. The volatiles were removed in vacuo and the residue partitioned between Saturated NaHCO₃ and ethylacetate. Concentration in vacuo of the organic phase yielded ethyl 3-nitro-4-(1H-pyrrol-1-yl)benzoate as a dark oil (3.70 g, 98%). LCMS (ES): >85% pure, m/z 261 [M+1]⁺.

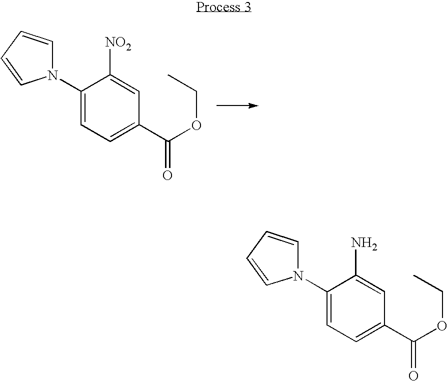

ethyl 3-nitro-4-(1H-pyrrol-1-yl)benzoate (1.0 eq, 4.39 g, 16.8 mmol) was dissolved in EtOH (20 ml) and BiCl3 (0.5 eq, 2.65 g, 8.4 mmol) The mixture was cooled to O° C. and NaBH₄ (4.0 eq, 2.55 g, 67.2 mmol) was added in small portions. The reaction was allowed to warm to room temperature. The reaction was quenched with HCl and neutralized with NaHCO₃. The material was extracted with EtOAc (2×). After drying over Na₂SO₄ and evaporation of the solvents, ethyl 3-amino-4-(1H-pyrrol-1-yl)benzoate was isolated as a beige semi-solid (3.54 g, 91% yield). LCMS (ES): >85% pure, m/z 231 [M+1]⁺.

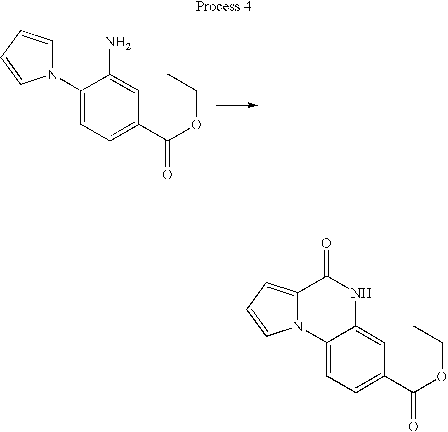

Ethyl 3-amino-4-(1H-pyrrol-1-yl)benzoate (1.0 eq, 3.36 g, 14.6 mmol) was combined with triphosgene (1.25 eq, 1.78 g, 6.08 mmol) in toluene (100 ml) and the mixture heated at reflux for 2 hours. After cooling, hexanes were added (50 ml) and the mixture filtered. The solid was dissolved in acetone and concentrated to a minimum. Trituration with water afforded ethyl 4-oxo-4,5-dihydropyrrolo[1,2-a]quinoxaline-7-carboxylate as a light beige solid (3.14 g, 84% yield). LCMS (ES): >90% pure, m/z 257 [M+1]⁺.

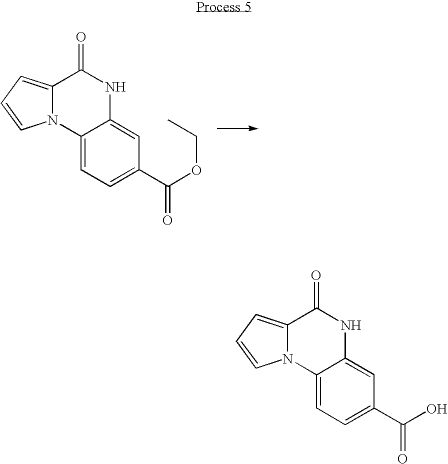

Ethyl 4-oxo-4,5-dihydropyrrolo[1,2-a]quinoxaline-7-carboxylate (100 mg) was combined with 6N aqueous NaOH (1 m) and stirred at 50° C. for several hours. The mixture was acidified with HCl and the solid filtered to afford 4-oxo-4,5-dihydropyrrolo[1,2-a]quinoxaline-7-carboxylic acid as brownish solid (60 mg). LCMS (ES): >85% pure, m/z 229 [M+1]⁺.

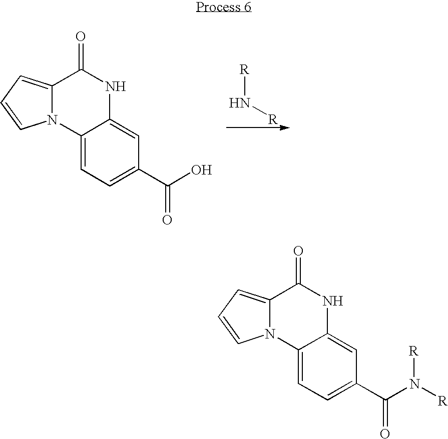

4-oxo-4,5-dihydropyrrolo[1,2-a]quinoxaline-7-carboxylic acid (1.0 eq, 35 mg, 0.154 mmol) was mixed with EDCI (2 eq, 59 mg, 0.307 mmol), HOBt (1.0 eq, 21 mg, 0.154 mmol), triethylamine (2.0 eq, 31 mg, .0.307 mmol) and an appropriate amine NHR₂ (2 eq) in dichloroethane (2 ml). The reaction was stirred at 70° C. for 4 hours. Solvents were removed, acetonitrile and water were added and the solution purified by preparative HPLC. The following compounds were prepared using this process and characterized by LCMS. Boc-protected amines were deprotected by subsequent treatment with DCM/TFA (1:1) and purified by preparative HPLC.

The following table (Table 1) contains some of the quinoxalinone intermediates made by the above-described methods, which are useful for the preparation of many compounds of Formula I and/or II.

TABLE 1
Intermediates for use in making compounds of the invention.

| | | LCMS |
|---|---|---|
| Structure | MW | [M + 1]+ |
| 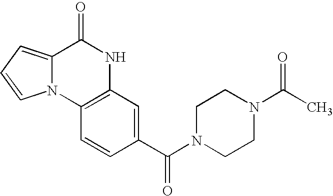 | 338.36 | 339 |
| 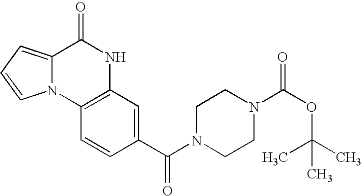 | 396.44 | 397 |
| 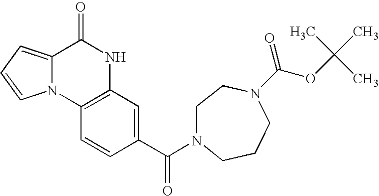 | 410.47 | 411 |
| 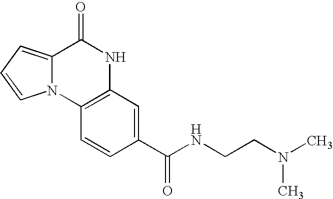 | 298.34 | 299 |
| 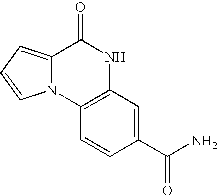 | 227.22 | 228 |
| 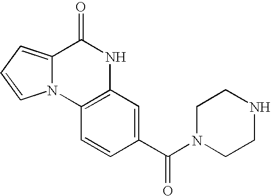 | 296.32 | 297 |
| 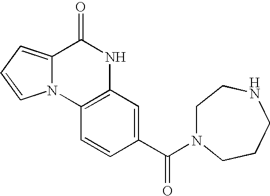 | 310.35 | 311 |
| 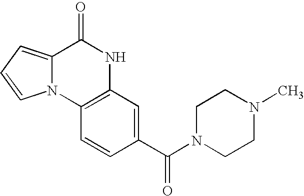 | 310.35 | 311 |

### Example 2

### Processes for Synthesizing Pyrrole Compounds

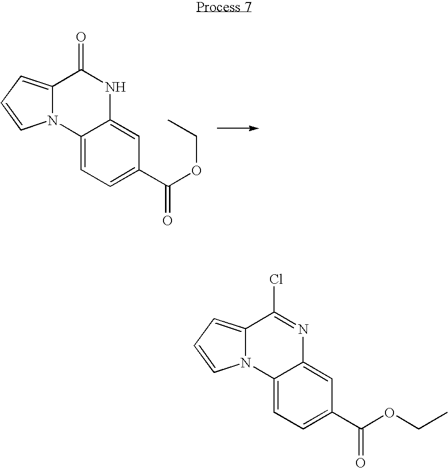

Ethyl 4-oxo-4,5-dihydropyrrolo[1,2-a]quinoxaline-7-carboxylate (150 mg) was heated with phosphorus oxichloride (2 ml) overnight. The reaction was diluted with CH₂Cl₂, washed with NaHCO₃, dried over Na₂SO₄ and the solvent removed in vacuo to gige 157 mg of ethyl 4-chloropyrrolo[1,2-a]quinoxaline-7-carboxylate. LCMS (ES): >85% pure, m/z 275 [M+1]⁺.

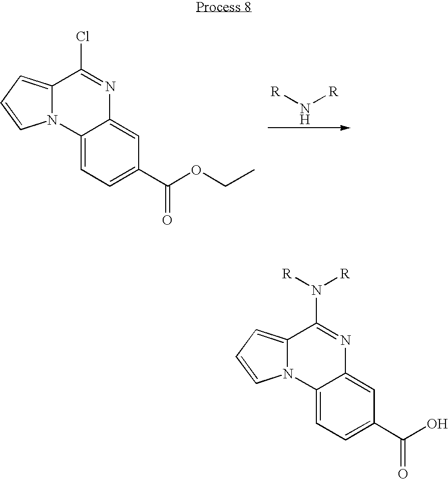

Ethyl 4-chloropyrrolo[1,2-a]quinoxaline-7-carboxylate (35 mg) was reacted with the appropriate amine HNR₂ (10 eq) in NMP (0.5 ml) using microwave heating at 120° C. for 10 min 6N NaOH (0.5 ml) was added and the mixture stirred at 50° C. for one hour. Acetonitrile and HCl were added and the solution purified by preparative HPLC.

### Example 3

### Processes for Synthesizing Pyrrole Compounds

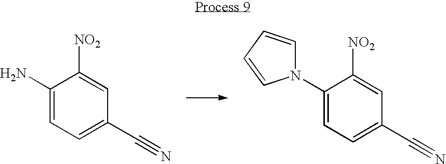

4-amino-3-nitrobenzonitrile (1.0 eq) was reacted with 2,5-dimethoxy-tetrahydrofuran (1.0 eq) in acetic acid. The reaction was heated in a microwave oven at 160° C. for 10 min. The solvent was evaporated and the residue purified by flash chromatography on silica gel (hexanes/EtOAc gradient). 3-nitro-4-(1H-pyrrol-1-yl)benzonitrile was obtained as an orange-yellow solid (59% yield). LCMS (ES): >95% pure, m/z 214 [M+1]⁺.

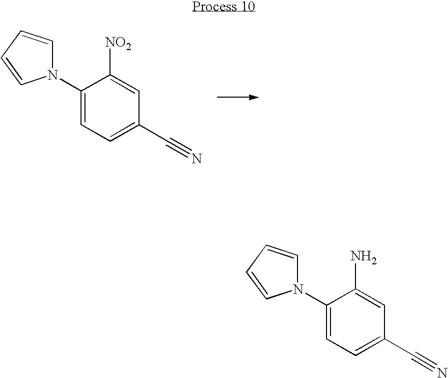

3-nitro-4-(1H-pyrrol-1-yl)benzonitrile (1.0 eq, 1.24 g, 5.81 mmol) was reacted with SnCl₂ (5.2 eq, 5.72 g, 30.16 mmol) in refluxing Ethanol (80 ml) under nitrogen for 35 min NaHCO₃ was added and the resulting solid was filtered off through celite. EtOH was concentrated. CH₂Cl₂ was added and the material purified by flash chromatography on silica gel (CH₂Cl₂/MeOH, 95.5/0.5%) to obtain 3-amino-4-(1H-pyrrol-1-yl)benzonitrile as an orange oil (0.56 g, 53% yield). LCMS (ES): >85% pure, m/z 184 [M+1]⁺.

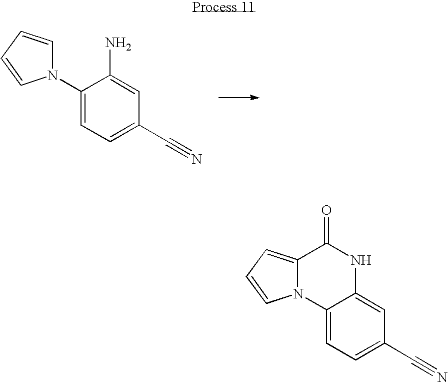

3-amino-4-(1H-pyrrol-1-yl)benzonitrile (1 eq) was reacted with triphosgene (1 eq) in toluene at reflux for 30 min Nitrogen was bubbled in the reaction to remove residual phosgene and the solid filtered and washed with hexanes. 4-oxo-4,5-dihydropyrrolo[1,2-a]quinoxaline-7-carbonitrile was isolated as a white solid (76% yield). LCMS (ES): >85% pure, m/z 210 [M+1]⁺.

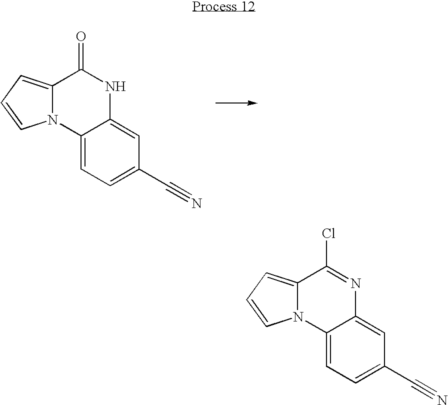

4-oxo-4,5-dihydropyrrolo[1,2-a]quinoxaline-7-carbonitrile (1.0 eq, 190 mg, 0.906 mmol) was suspended in toluene (4 ml) and mixed with DIEA (0.8 eq, 126 uL, 0.725 mmol) and POCl3 (1.2 eq, 100 ul, 1.09 mmol) and stirred at 120° C. for 2.5 hours. Water and ice were added and material was extracted with CH2Cl2. After evaporation of the solvents, the material was triaturated in hexanes/AcOEt and filtered to give 4-chloropyrrolo[1,2-a]quinoxaline-7-carbonitrile as a colorless solid (143 mg, 70% yield). LCMS (ES): >95% pure, m/z 228 [M+1]+.

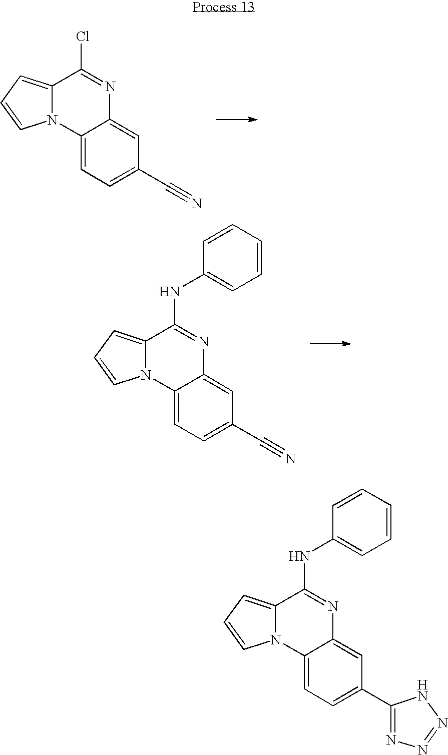

4-chloropyrrolo[1,2-a]quinoxaline-7-carbonitrile (1.0 eq, 31 mg, 0.137 mmol) was mixed with aniline (8 eq, 0.1 ml, 1.1 mmol) in NMR (0.1 ml) and the mixture heated in a microwave oven at 120° C. for 10 min. The material was crashed out using hexanes/EtOAc to afford a solid (12.2 mg, 31% yield). LCMS (ES) m/z 285 [M+1]+.

The material was treated with NaN₃ (5.3 eq, 15 mg, 0.23 mmol) and NH4Cl (4.4 eq, 10.4 mg, 0.19 mmol) in DMF (0.5 ml) at 120° C. for 1 hour. Additional reagents were added to complete the reaction after extra 1.5 hours.

Water and HCl were added and the solid was filtered. Trituration in hexanes/EtOAc afforded N-phenyl-7-(1H-tetrazol-5-yl)pyrrolo[1,2-a]quinoxalin-4-amine as white solid (5.5 mg, 39% yield). LCMS (ES): >95% pure, m/z 328 [M+1]+.

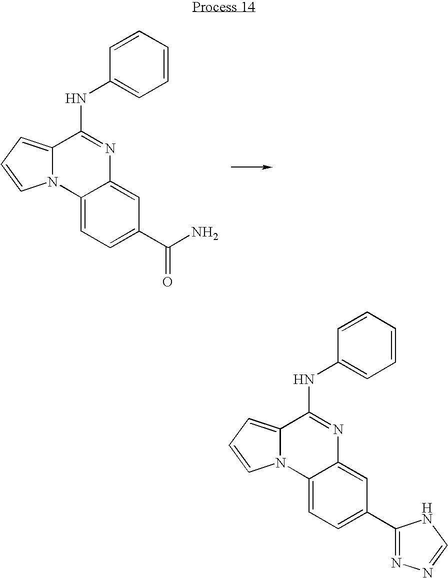

4-(phenylamino)pyrrolo[1,2-a]quinoxaline-7-carboxamide (1.0 eq, 18.4 mg, 0.06 mmol) was heated in DMF-DMA for 2.5 hours. The volatiles were removed in vacuo and the residue was dissolved in acetic acid (0.28 ml). Hydrazine hydrate was added and the mixture heated at 110° C. for 30 minutes. Water was added and the resulting solid was extracted with CH₂Cl₂. After drying over Na₂SO₄ and evaporation of the solvents, the material was purified by flash chromatography to afford 7.4 mg of N-phenyl-7-(4H-1,2,4-triazol-3-yl)pyrrolo[1,2-a]quinoxalin-4-amine. LCMS (ES): >95% pure, m/z 327 [M+1]⁺

The molecules described in the following table (Table 2) were prepared using reactions similar to the ones exemplified in processes 1 to 14, and the reaction schemes shown above. All compounds were characterized by LCMS.

TABLE 2
Exemplary compounds of the invention.

| | | LCMS |
|---|---|---|
| Structure | MW | [M + 1]+ |
| 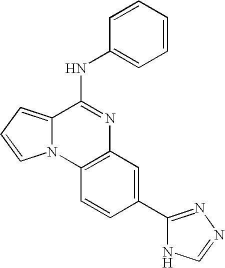 | 326.35 | 327 |
| 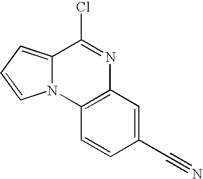 | 227.65 | 228 |
| 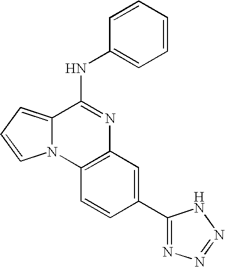 | 327.34 | 328 |
| 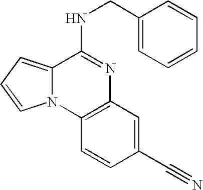 | 298.34 | 299 |
| 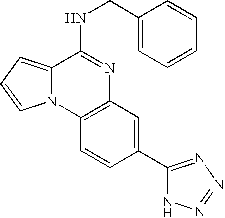 | 341.37 | 342 |
| 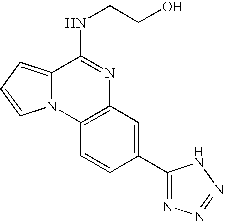 | 295.30 | 296 |
| 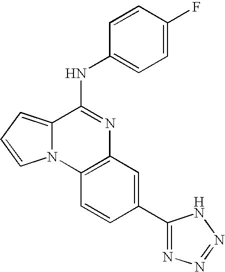 | 345.33 | 346 |
| 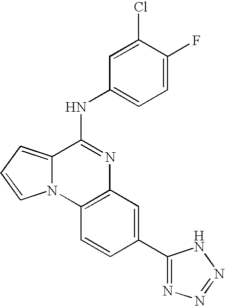 | 379.78 | 380 |
| 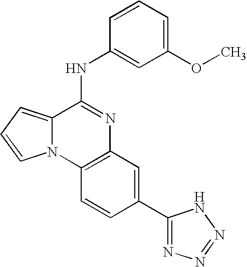 | 357.37 | 358 |
| 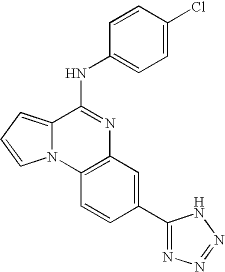 | 361.79 | 362 |
| 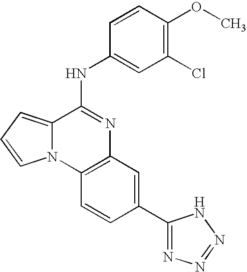 | 391.81 | 392 |
| 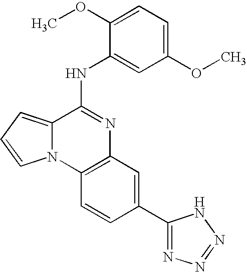 | 387.39 | 388 |
| 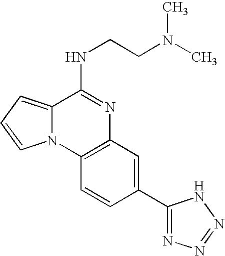 | 322.37 | 323 |
| 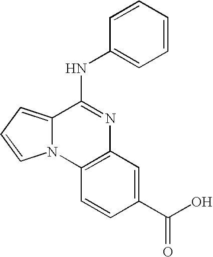 | 303.31 | 304 |
| 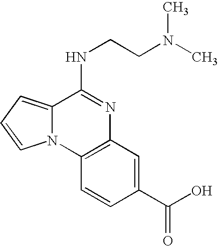 | 298.34 | 299 |
| 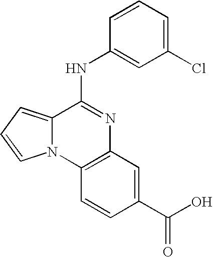 | 337.76 | 338 |
| 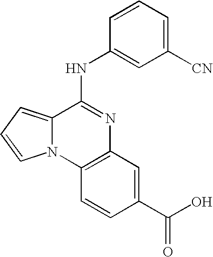 | 328.32 | 329 |
| 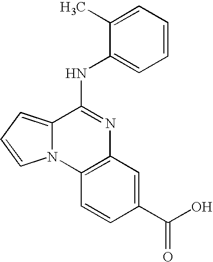 | 317.34 | 318 |
| 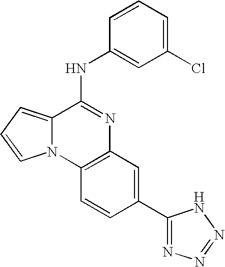 | 361.79 | 362 |
| 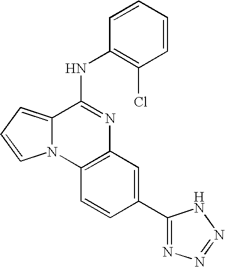 | 361.79 | 362 |
| 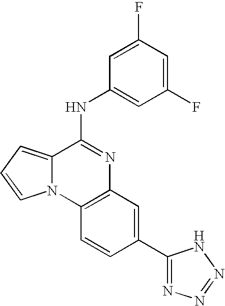 | 363.32 | 364 |
| 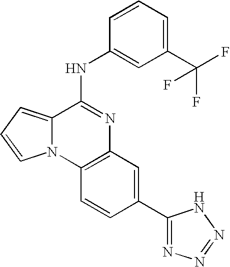 | 395.34 | 396 |
| 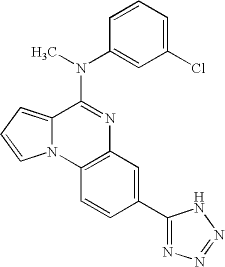 | 375.81 | 376 |
| 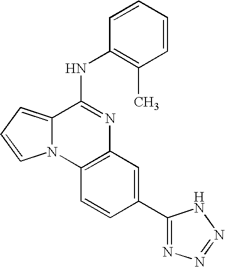 | 341.37 | 342 |
| 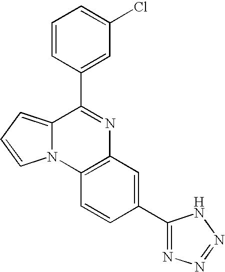 | 346.77 | 347 |
| 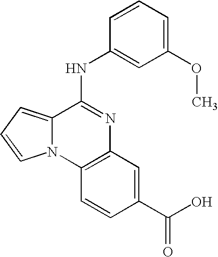 | 333.34 | 334 |
| 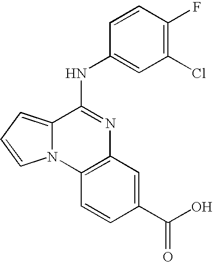 | 355.75 | 356 |
| 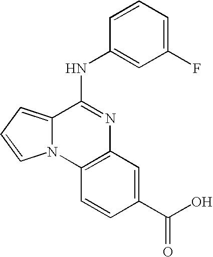 | 321.31 | 322 |
| 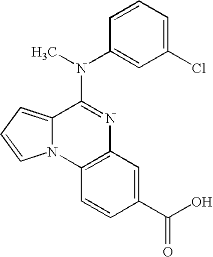 | 351.79 | 352 |
| 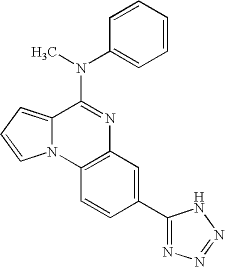 | 341.37 | 342 |
| 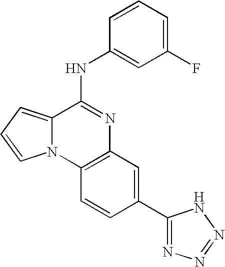 | 345.33 | 346 |
| 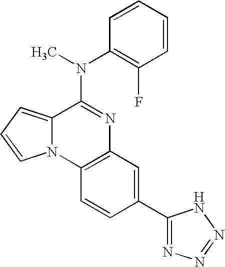 | 359.36 | 360 |
| 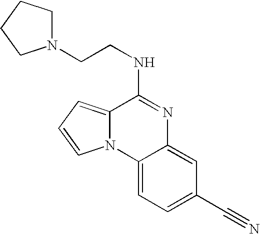 | 305.38 | 306 |
| 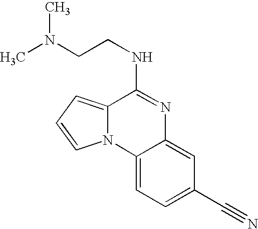 | 279.34 | 280 |
| 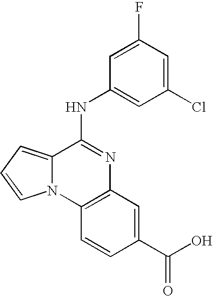 | 355.75 | 356 |
| 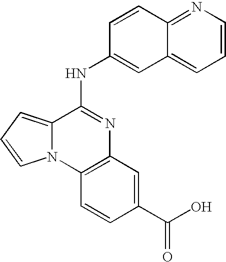 | 354.36 | 355 |
| 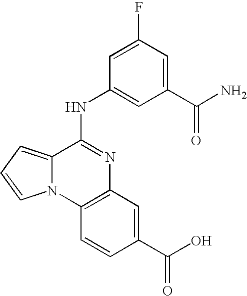 | 364.33 | 365 |
| 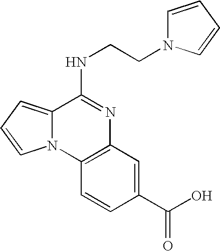 | 320.35 | 321 |
| 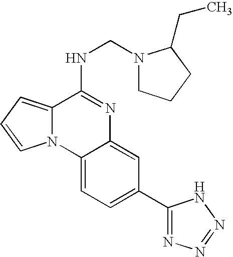 | 362.43 | 363 |
| 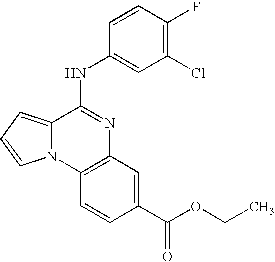 | 383.80 | 384 |
| 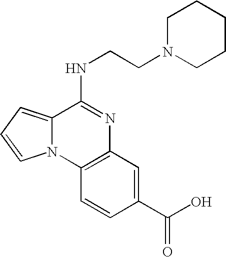 | 338.40 | 339 |
| 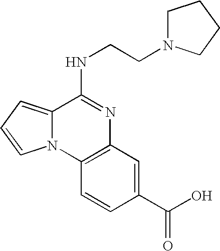 | 324.38 | 325 |
| 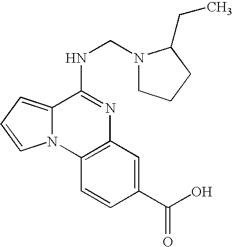 | 338.40 | 339 |
| 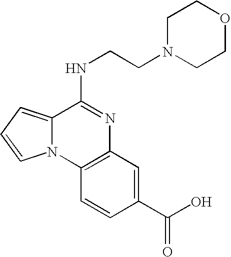 | 340.38 | 341 |
| 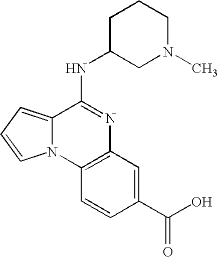 | 324.38 | 325 |
| 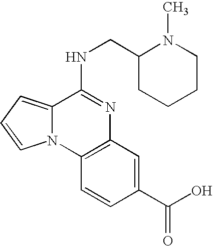 | 338.40 | 339 |
| 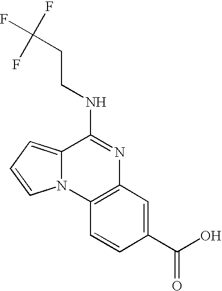 | 323.27 | 324 |
| 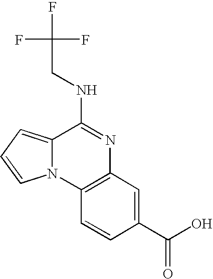 | 309.24 | 310 |
| 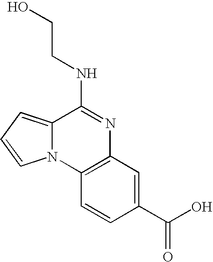 | 271.27 | 272 |
| 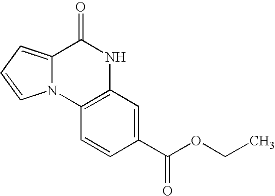 | 256.26 | 257 |
| 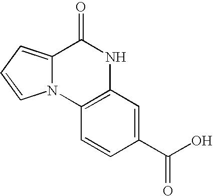 | 228.20 | 229 |
| 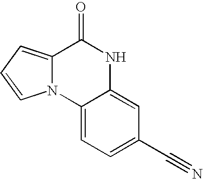 | 209.20 | 210 |
| 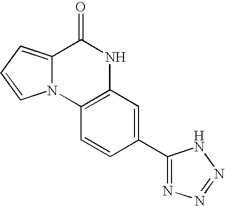 | 252.23 | 253 |

### Example 4

### Processes for the Synthesis of Pyrrole Compounds

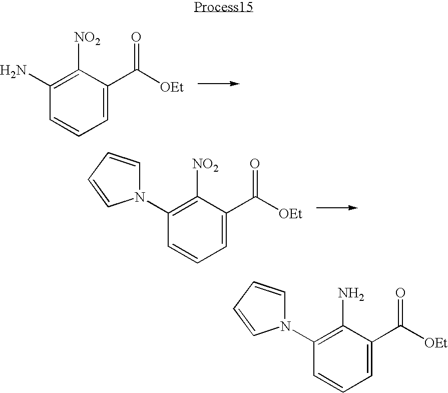

Ethyl 3-amino-2-nitrobenzoate [prepared according to the U.S. Pat. No. 6,166,219] (1.0 eq, 5 g, 23.8 mmol) and 2,5-dimethoxytetrahydrofuran (1.1 eq, 3.4 mL, 26.2 mmol) were mixed in glacial acetic acid (16 mL). The mixture was fitted with a condenser and placed in a 120° C. oil bath. After 1 h, the reaction was concentrated in vacuo to give a reddish brown oil which was partitioned between ethyl acetate (250 mL) and saturated NaHCO3 (250 mL). The organic layer was dried over MgSO4, filtered and concentrated in vacuo. The residue was purified via flash column chromatography (10-15% EtOAc/hexanes) to afford ethyl 2-nitro-3-(1H-pyrrol-1-yl)benzoate (5.45 g, 88%) as a bright yellow solid. 1H NMR (CDCl₃, 400 MHz) δ: 8.04 (dd, 1H, J=7.2, 1.8 Hz), 7.60-7.67 (m, 2H), 6.82 (dd, 2H, J=2.4, 2.4 Hz), 6.34 (dd, 2H, J=2.4, 2.4 Hz), 4.40 (q, 2H, J=7.2 Hz), 1.38 (t, 3H, J=7.2 Hz). 13C NMR (CDCl3, 100 MHz) δ: 162.6, 133.6, 131.8, 130.6, 129.7 (two carbons), 124.4, 122.0, 111.0, 62.6, 13.7. LCMS (ES): >95% pure, m/z 261 [M+1]+.

Ethyl 2-nitro-3-(1H-pyrrol-1-yl)benzoate (6.68 g, 25.7 mmol) was dissolved in ethanol (200 mL, denatured with 5% methanol and 5% isopropanol). The solution was degassed with a stream of nitrogen for 15 min, then 10% Pd/C (500 mg, Degussa type) was added. The reaction was purged with H2 (40 psi), then evacuated. The flask was refilled with H2 (40 psi) and shaken on a Parr shaker. When the pressure of H2 fell to 20 psi, the flask was repressurized to 40 psi. After 4.5 h, the solution was gravity filtered and the Pd/C was washed with ethanol (3×20 mL). The filtrate was concentrated in vacuo to a green oil. Flash column chromatography (2.5% EtOAc/hexanes) furnished ethyl 2-amino-3-(1H-pyrrol-1-yl)benzoate (5.39 g, 91%) as a pale yellow oil. 1H NMR (CDCl3, 400 MHz) δ: 7.96 (dd, 1H, J=8.0, 1.6 Hz), 7.30 (dd, 1H, J=8.0, 1.6 Hz), 6.81 (dd, 2H, J=2.0, 2.0 Hz), 6.69 (dd, 1H, J=8.0, 8.0 Hz), 6.38 (dd, 2H, J=2.0, 2.0 Hz), 5.80-6.00 (bs, 2H), 4.38 (q, 2H, J=7.2 Hz), 1.42 (t, 3H, J=7.2 Hz). 13C NMR (CDCl3, 100 MHz) δ: 167.8, 146.7, 131.7, 130.9, 128.0, 121.7, 114.9, 111.9, 109.7, 60.6, 14.3. LCMS (ES): >95% pure, m/z 185.

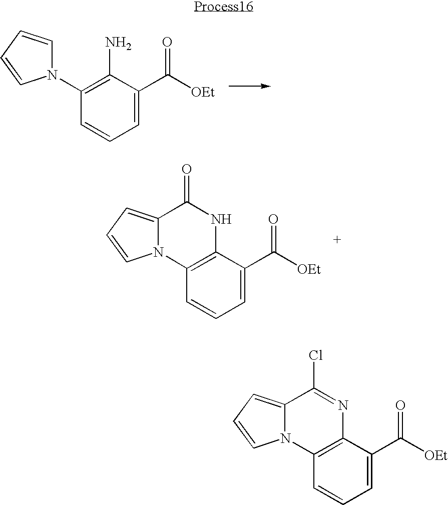

Ethyl 2-amino-3-(1H-pyrrol-1-yl)benzoate (1.0 eq, 3.99 g, 17.3 mmol) and triphosgene (1.5 eq, 7.7 g, 26 mmol) were dissolved in anhydrous toluene (115 mL), then placed in a 120° C. oil bath. Triphosgene (3.8 g) was added every hour at reflux. After 6 h, the solution was cooled to 23° C. and then poured into cold hexanes (350 mL). The solution stirred for 5 min and then the light brown precipitate, ethyl 4-oxo-4,5-dihydropyrrolo[1,2-a]quinoxaline-6-carboxylate (591 mg, 13%), was filtered off. LCMS (ES): >95% pure, m/z 257 [M+1]+. The filtrate was concentrated in vacuo and the residue was purified via flash column chromatography (1-2% MeOH/CH2Cl2) to give ethyl 4-chloropyrrolo[1,2-a]quinoxaline-6-carboxylate (3.48 g, 73%) as a maize colored solid. 1H NMR (CDCl3, 400 MHz) δ: 7.94 (dd, 1H, J=2.8, 1.6 Hz), 7.90 (dd, 1H, J=8.0, 1.6 Hz), 7.66 (dd, 1H, J=8.0, 1.2 Hz), 7.50 (dd, 1H, J=8.0, 8.0 Hz), 7.03 (dd, 1H, J=4.0, 1.6 Hz), 6.87 (dd, 1H, J=4.0, 2.8 Hz), 4.51 (q, 2H, J=7.2 Hz), 1.42 (t, 3H, J=7.2 Hz). 13C NMR (CDCl₃, 100 MHz) δ: 167.0, 145.9, 132.7, 132.3, 127.3, 127.2, 125.5, 123.8, 116.3, 116.2, 114.5, 109.1, 61.7, 14.3. LCMS (ES): >95% pure, m/z 275 [M+1]+.

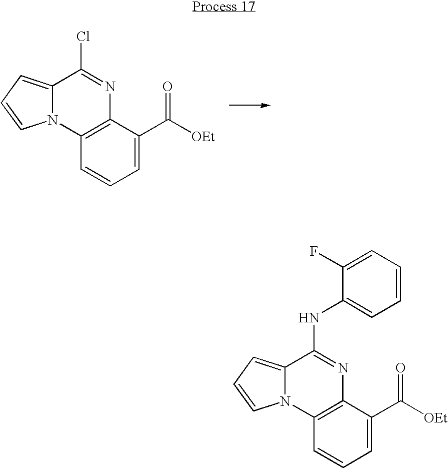

2-fluoroaniline (2 eq, 717 μL, 7.42 mmol) was added to a solution of ethyl 4-chloropyrrolo[1,2-a]quinoxaline-6-carboxylate (1 eq, 1.02 g, 3.7 mmol) dissolved in anhydrous DMF (25 mL). The reaction was placed in a 120° C. oil bath. After 40 min, the reaction was complete by LCMS. The solution was cooled to 23° C., then poured into H₂O (100 mL). The aqueous solution was allowed to stir for 10 min, and then filtered. The solid was purified via flash column chromatography (10-20% EtOAc/hexanes) to provide ethyl 4-(2-fluorophenylamino)pyrrolo[1,2-a]quinoxaline-6-carboxylate (773 mg, 60%) as a pink/orange solid. LCMS (ES): >95% pure, m/z 350 [M+1]⁺.

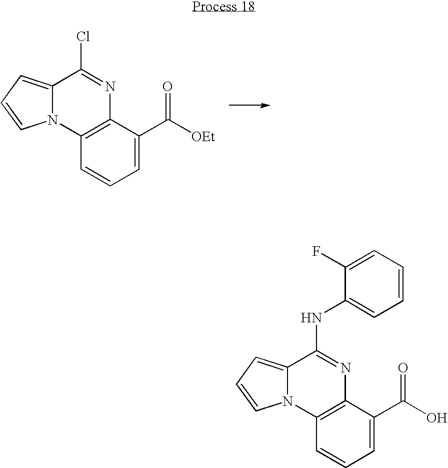

2-fluoroaniline (2 eq, 703 μL, 7.28 mmol) was added to a solution of ethyl 4-chloropyrrolo[1,2-a]quinoxaline-6-carboxylate (1 eq, 1.0 g, 3.64 mmol) dissolved in anhydrous NMP (4.8 mL). The vial was sealed and placed in the microwave at 140° C. After 10 min, the reaction was complete by LCMS. The solution was cooled to 23° C., then poured into H₂O (150 mL). The solid was purified via flash column chromatography (2.5-10% MeOH/dichloromethane) to yield 4-(2-fluorophenylamino)pyrrolo[1,2-a]quinoxaline-6-carboxylate (382 mg, 33%) as a light brown solid. LCMS (ES): >90% pure, m/z 322 [M+1]⁺.

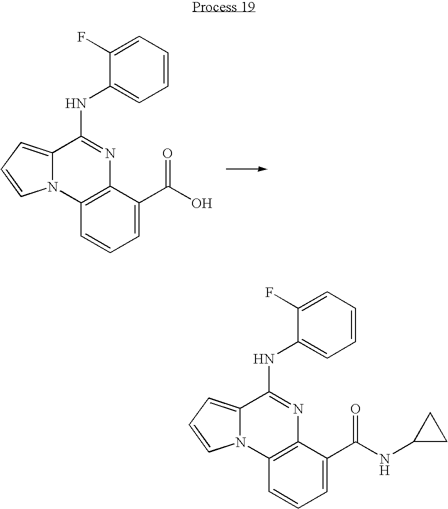

4-(2-fluorophenylamino)pyrrolo[1,2-a]quinoxaline-6-carboxylate (1 eq, 30 mg, 0.09 mmol) was suspended in 1,4-dioxane (0.5 mL). Cyclopropylamine (8 eq, 52 μL, 0.75 mmol), EDCI.HCl (2 eq, 36 mg, 0.19 mmol), and HOBt (2 eq, 25 mg, 0.19 mmol) were added in one portion in sequential order. DIEA (8 eq, 128 μL, 0.75 mmol) was added rapidly dropwise. The mixture was stirred for 2 min, then placed in an 80° C. oil bath. After 2.5 h, the solution was cooled to 23° C. and then cyclopropylamine (4 eq, 26 μL, 0.37 mmol), EDCI.HCl (1 eq, 18 mg, 0.09 mmol), and HOBt (1 eq, 12 mg, 0.09 mmol) were again added in one portion in sequential order. DIEA (4 eq, 64 μL, 0.37 mmol) was added rapidly dropwise and the solution was placed in an 80° C. oil bath. After 1 h, the solution was cooled to 23° C. and diluted with methanol (0.5 mL). The crude residue was purified via preparative thin layer chromatography (3% methanol/dichloromethane) to provide 4-oxo-4,5-dihydropyrrolo[1,2-a]quinoxaline-6-carboxamide (12 mg, 36%). LCMS (ES): >95% pure, m/z 361 [M+1]⁺.

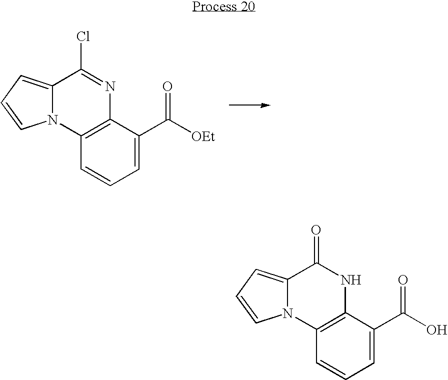

4-chloro-6-(4H-1,2,4-triazol-3-yl)pyrrolo[1,2-a]quinoxalin (1.5 g, 5.46 mmol) was suspended in 6M NaOH. The reaction was stirred vigorously and placed in an 80° C. oil bath for 2 d. The white suspension was poured into water (75 mL) and the solution was cooled to 0° C. The pH was adjusted to 1-2 by dropwise addition of 6M HCl. The yellow precipitate was filtered off, then triturated twice with acetone (1×40 mL, 1×20 mL). The white solid was filtered and dried under high vacuum to afford 4-oxo-4,5-dihydropyrrolo[1,2-a]quinoxaline-6-carboxylic acid (1.14 g, 91%). LCMS (ES): >90% pure, m/z 229 [M+1]⁺.

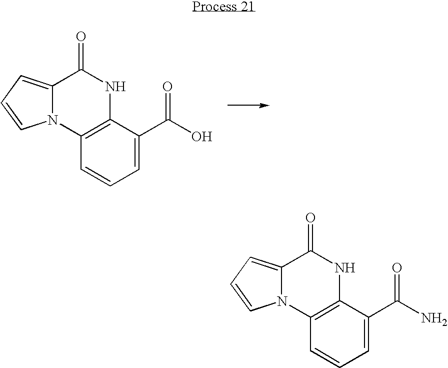

4-oxo-4,5-dihydropyrrolo[1,2-a]quinoxaline-6-carboxylic acid (1 eq, 654 mg, 2.86 mmol) was suspended in 1,4-dioxane (15 mL). Ammonium chloride (8 eq, 1.23 g, 22.9 mmol), EDCI.HCl (2 eq, 1.1 g, 5.73 mmol), and HOBt (2 eq, 775 mg, 5.73 mmol) were added in one portion in sequential order. DIEA (8 eq, 3.9 mL, 22.9 mmol) was added rapidly dropwise. The mixture was stirred for 2 min, then placed in an 80° C. oil bath. After 1 h, the solution was cooled to 23° C. and then ammonium chloride (2 eq, 307 mg, 5.73 mmol), EDCI.HCl (0.5 eq, 275 mg, 1.43 mmol), and HOBt (0.5 eq, 194 mg, 1.43 mmol) were again added in one portion in sequential order. DIEA (2 eq, 975 μL, 5.73 mmol) was added rapidly dropwise and the solution was placed in an 80° C. oil bath. After 1 h, the solution was cooled to 23° C., then poured into water (100 mL). The precipitate was collected and triturated with 10% MeOH/ether (20 mL). 4-oxo-4,5-dihydropyrrolo[1,2-a]quinoxaline-6-carboxamide (527 mg, 81%) was collected by filtration as an off white solid. LCMS (ES): >90% pure, m/z 228 [M+1]⁺.

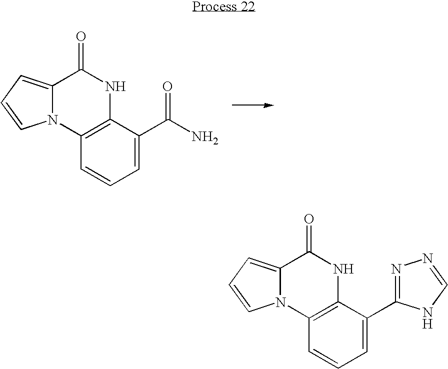

4-oxo-4,5-dihydropyrrolo[1,2-a]quinoxaline-6-carboxamide (527 mg, 2.32 mmol) was suspended in dimethylformamide dimethylacetal (20 mL). The suspension was placed in an 80° C. oil bath. After 1.5 h, the bright yellow suspension was concentrated in vacuo and used crude for the next reaction. The crude solid was dissolved in glacial acetic acid (11.5 mL). NH₂NH₂. H₂O (2.3 mL) was added dropwise. The reaction was placed in an 80° C. oil bath for 1.5 h. The solution was cooled to 23° C., then poured into water (100 mL). The precipitate 6-(4H-1,2,4-triazol-3-yl)pyrrolo[1,2-a]quinoxalin-4(5H)-one (519 mg, 89% over two steps) was collected by filtration. LCMS (ES): >95% pure, m/z 252 [M+1]⁺.

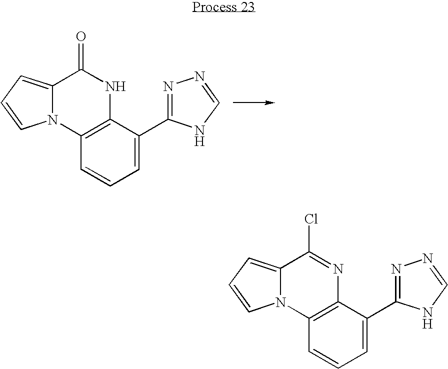

6-(4H-1,2,4-triazol-3-yl)pyrrolo[1,2-a]quinoxalin-4(5H)-one (1 eq, 519 mg, 2.07 mmol) was wetted with CH₃CN (2.3 mL). Triethylamine (1.05 eq, 302 μL, 2.17 mmol) was added in one portion, and then POCl₃ (4 eq, 756 μL, 8.26 mmol) was added dropwise. The suspension was placed in a 100° C. oil bath. LCMS indicated the completion of the reaction after 2 h. The reaction mixture was added dropwise to a solution of CH₃CN (50 mL), triethylamine (50 mL), and methanol (25 mL) cooled to 0° C. The solution was poured into water (125 mL) and the precipitate was filtered off. The product was dried under high vacuum to yield 4-chloro-6-(4H-1,2,4-triazol-3-yl)pyrrolo[1,2-a]quinoxalin (467 mg, 84%) as a yellow solid. LCMS (ES): >85% pure, m/z 270 [M+1]⁺.

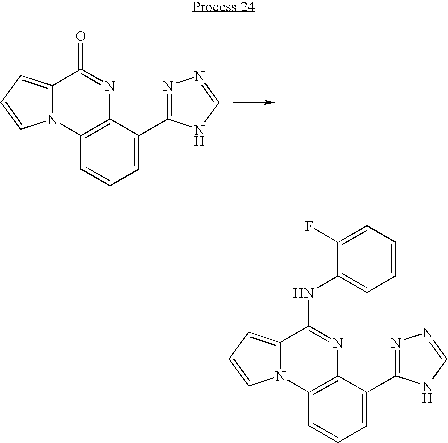

In a microwave vial, 4-chloro-6-(4H-1,2,4-triazol-3-yl)pyrrolo[1,2-a]quinoxalin (1 eq, 25 mg, 0.09 mmol) was suspended in anhydrous NMP (125 μL). 2-fluoroaniline (2 eq, 18 μL, 0.18 mmol) was added in one portion. The vial was sealed and heated to 140° C. under microwave irradiation for 10 min. The solution was diluted with methanol (0.5 mL) and filtered through a 0.45 μm PTFE syringe filter. The filtrate was purified by preparative LCMS to afford N-(2-fluorophenyl)-6-(4H-1,2,4-triazol-2-yl)pyrrolo[1,2-a]quinoxalin-4-amine (2 mg, 6%). LCMS (ES): >95% pure, m/z 345 [M+1]⁺.

The molecules described in the following table (Table 3) were prepared using chemistries similar to the one exemplified in processes 15 through 24. All compounds were characterized by LCMS.

TABLE 3
Exemplary Compounds of the Invention

| | | LCMS |
|---|---|---|
| | | m/z |
| Structure | MW | [M + 1]+ |
| 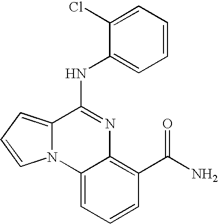 | 336.8 | 337 |
| 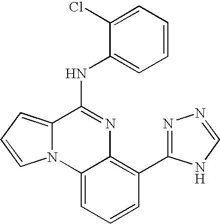 | 360.8 | 361 |
| 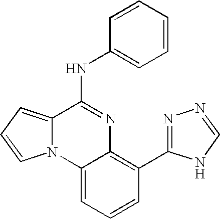 | 326.4 | 327 |
| 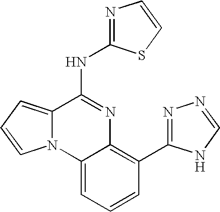 | 333.4 | 334 |
| 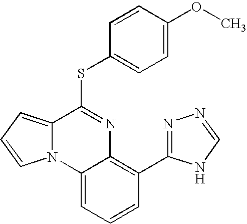 | 373.4 | 374 |
| 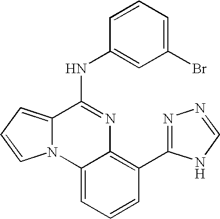 | 405.3 | 406 |
| 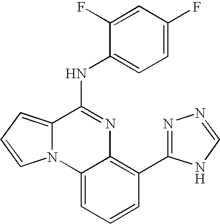 | 362.3 | 363 |
| 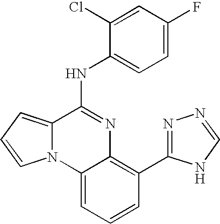 | 378.8 | 379 |
| 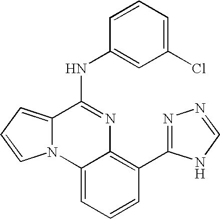 | 360.8 | 361 |
| 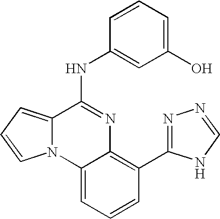 | 342.4 | 343 |
| 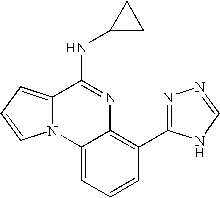 | 290.3 | 291 |
| 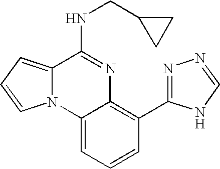 | 304.3 | 305 |
| 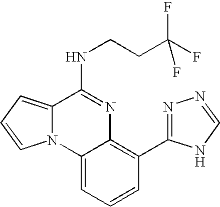 | 346.3 | 347 |
| 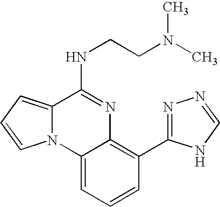 | 321.4 | 322 |

### Example 5

### Processes for Synthesis of Imidazole Compounds

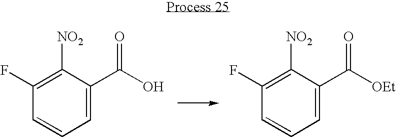

Commercially available (Apollo) 3-fluoro-2-nitrobenzoic acid (1.93 g, 10.42 mmol) was dissolved in absolute ethanol (20 mL). Sulfuric acid (0.2 mL) was added and the reaction was placed in a 95° C. bath. After 18 h, the solution was cooled to 23° C. and poured into sat. NaHCO₃ (150 mL). The precipitate was filtered and dried under high vacuum to provide ethyl 3-fluoro-2-nitrobenzoic acid (1.51 g, 68%) as a light brown solid. ¹H NMR (CDCl₃, 400 MHz) δ: 7.83 (ddd, 1H, J=7.6, 1.2, 1.2 Hz), 7.58 (ddd, 1H, J=7.6, 7.6, 4.8 Hz), 7.46 (ddd, 1H, J=8.8, 8.8, 1.2 Hz), 4.39 (q, 2H, J=7.2 Hz), 1.37 (t, 3H, J=7.2 Hz).

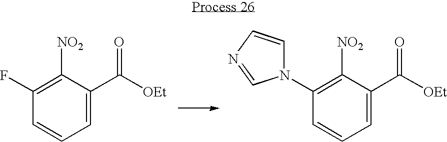

Ethyl 3-fluoro-2-nitrobenzoic acid (0.5 g, 2.35 mmol) was dissolved in anhydrous acetonitrile (4 mL). Imidazole (0.4 g, 5.85 mmol) and then potassium carbonate (0.81 g, 5.85 mmol) were added in one portion. The reaction was placed in a 95° C. oil bath. After 2.5 h, the reaction was poured into water (25 mL). The precipitate was filtered off and dried under high vacuum to give ethyl 3-(1H-imidazol-1-yl)-2-nitrobenzoate (352 mg, 57%) as an off white solid. ¹H NMR (CDCl₃, 400 MHz) δ: 8.17 (dd, 1H, J=8.0, 1.2 Hz), 7.72 (dd, 1H, J=7.6, 7.6 Hz), 7.61 (m, 2H), 7.20 (m, 1H), 7.10 (dd, 1H, 1.2, 1.2 Hz), 4.40 (q, 2H, J=7.2 Hz), 1.37 (t, 3H, J=7.2 Hz).

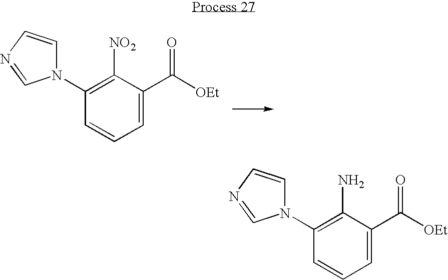

Ethyl 3-(1H-imidazol-1-yl)-2-nitrobenzoate (387 mg, 1.48 mmol) was suspended in ethanol (60 mL, denatured with 5% MeOH and 5% IPA). The solution was degassed with a stream of nitrogen for 10 min, then 10% Pd/C (95 mg, Degussa type) was added. On a Parr apparatus, the solution was filled with H₂ gas (40 psi), then purged. The flask was repressurized with H₂ gas (40 psi) and shaken for 1 h. The Pd/C was filtered off and washed with ethanol (10 mL). The filtrate was concentrated in vacuo to give an off white solid. The solid was triturated with ether and filtered. The filtrate was concentrated and purified by preparative thin layer chromatography (5% methanol/dichloromethane) which was combined with the solid to yield ethyl 2-amino-3-(1H-imidazol-1-yl)benzoate (209 mg, 61%). ¹H NMR (CDCl₃, 400 MHz) δ: 8.01 (dd, 1H, J=8.0, 1.2 Hz), 7.62 (s, 1H), 7.26 (m, 2H), 7.10 (s, 1H), 6.72 (dd, 1H, 8.0, 8.0 Hz), 5.82 (m, 2H), 4.38 (q, 2H, J=7.2 Hz), 1.41 (t, 3H, J=7.2 Hz).

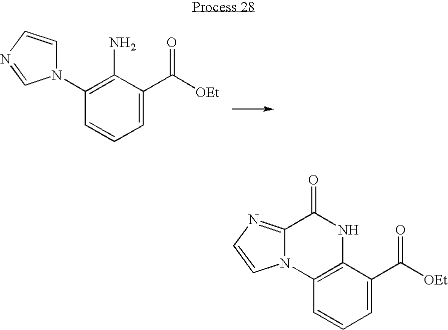

Ethyl 2-amino-3-(1H-imidazol-1-yl)benzoate (1 eq, 209 mg, 0.9 mmol) was dissolved in 1,2-dichlorobenzene (7 mL). Carbonyldiimidazole (1.2 eq 176 mg, 1.09 mmol) was added and the reaction was heated to reflux. After 1.5 h, carbonyldiimidazole (176 mg) was added and the solution refluxed for two additional hours. The solution was cooled to 23° C. and the crude reaction mixture was filtered over a pad of silica. Elution with 100% dichloromethane separated the solvent from the product, which eluted with 5% methanol/dichloromethane. The solid was further purified via flash column chromatography (3% methanol/dichloromethane) to afford ethyl 4-oxo-4,5-dihydroimidazo[1,2-a]quinoxaline-6-carboxylate (120 mg, 52%) as an off white solid. LCMS (ES): >95% pure, m/z 258 [M+1]⁺.

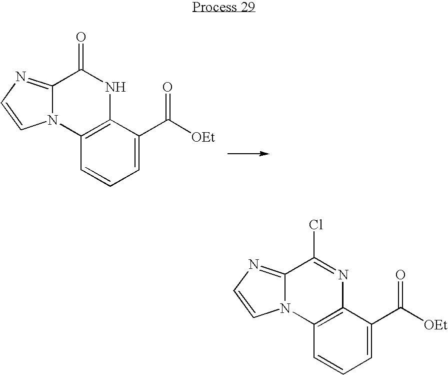

Ethyl 4-oxo-4,5-dihydroimidazo[1,2-a]quinoxaline-6-carboxylate (1 eq, 120 mg, 0.47 mmol) was wetted with acetonitrile (0.5 mL). Triethylamine (1.05 eq, 68 μL, 0.49 mmol) and then phosphorus oxychloride (4 eq, 171 μL, 1.87 mmol) were added. The suspension was placed in a 100° C. oil bath. After 2 h, the solution was cooled to 23° C. and added dropwise to a solution of CH₃CN (5 mL), triethylamine (5 mL), and methanol (2.5 mL) cooled to 0° C. The solution was poured into water (50 mL) and the precipitate was filtered off and dried under high vacuum to provide crude ethyl 4-chloroimidazo[1,2-a]quinoxaline-6-carboxylate (127 mg, 99%) which was used in the next reaction without further purification. LCMS (ES): >85% pure, m/z 276 [M+1]⁺.

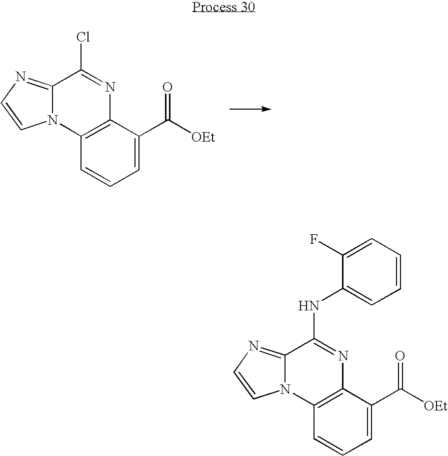

Ethyl 4-chloroimidazo[1,2-a]quinoxaline-6-carboxylate (1 eq, 127 mg, 0.46 mmol) was dissolved in anhydrous DMF (2.3 mL). 2-fluoroaniline (2 eq, 90 μL, 0.93 mmol) was added. The reaction was placed into a 120° C. oil bath. After 1 h, the solution was diluted with water (25 mL) and extracted with EtOAc (2×20 mL). The combined organics were washed with brine (1×50 mL), dried over MgSO₄, filtered, and concentrated in vacuo. The crude residue was purified by preparative thin layer chromatography (30% EtOAc/hexanes) to furnish ethyl 4-(2-fluorophenylamino)imidazo[1,2-a]quinoxaline-6-carboxylate (36 mg, 22% over two steps). LCMS (ES): >95% pure, m/z 351 [M+1]⁺.

### Example 6

### Processes for Synthesis of Imidazole Compounds

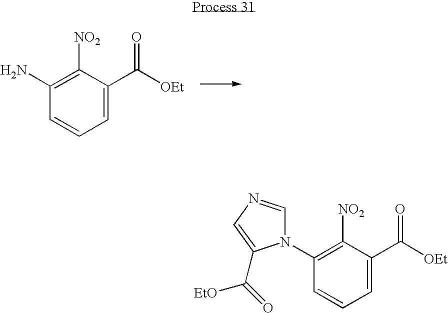

The following process was adapted from a similar process according to Chen et al., International Publication WO 2004/078714. Ethyl 3-amino-2-nitrobenzoate (1.0 eq, 2.5 g, 11.9 mmol) was dissolved in methanol (48 mL). Ethyl glyoxylate (4 eq, 9.71 g, 47.6 mmol, 50% wt in toluene) was added in one portion and the reaction was heated to reflux. After 15 h, a Dean-Stark trap was attached and half of the solvent was removed. Methanol (24 mL) was added, then half the solvent was removed again. Additional ethyl glyoxylate (2 mL) and methanol (24 mL) were added and the reaction was returned to reflux for 1 h. The solvent was removed in vacuo and used without further purification. The residue was dissolved in ethanol (150 mL). Tosylmethyl isocyanide (TosMIC, 1.3 eq, 6.27 g, 32.1 mmol) and K₂CO₃ (1.5 eq, 3.69 g, 26.7 mmol) were added. The solution was heated to 50° C. for 3.5 h. The solution was concentrated in vacuo, then diluted with water (75 mL) and extracted with EtOAc (3×75 mL). The organics were washed with brine (200 mL), dried over MgSO₄, filtered and concentrated in vacuo to an orange oil. Purification via flash column chromatography (40-60% EtOAc/hexanes) provided ethyl 1-[3-(ethoxycarbonyl)-2-nitrophenyl]-1H-imidazole-5-carboxylate (589 mg, 15% over two steps). LCMS (ES): >95% pure, m/z 334 [M+1]⁺.

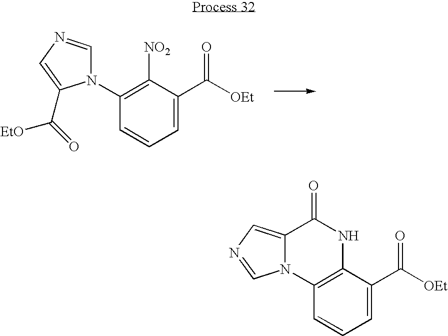

Ethyl 1-[3-(ethoxycarbonyl)-2-nitrophenyl]-1H-imidazole-5-carboxylate (1 eq, 589 mg, 1.76 mmol) was dissolved in glacial HOAc (6 mL) and iron metal (592 mg, 10.6 mmol) was added. The reaction was placed in a 105° C. bath. After 0.5 h, the reaction was cooled to 23° C. and diluted with water (15 mL) and the precipitate was filtered off. The solid was triturated with 2-propanol and then hexanes to afford ethyl 4-oxo-4,5-dihydroimidazo[1,5-a]quinoxaline-6-carboxylate (124 mg, 27%) as a tan solid. LCMS (ES): >95% pure, m/z 258 [M+1]+.

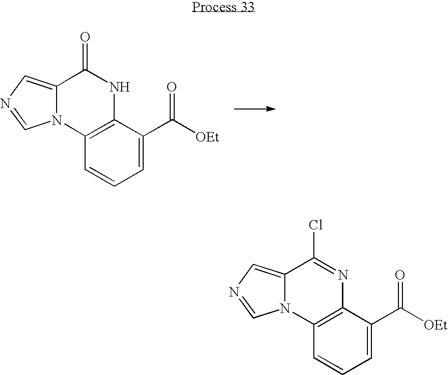

Ethyl 4-oxo-4,5-dihydroimidazo[1,5-a]quinoxaline-6-carboxylate (1 eq, 120 mg, 0.47 mmol) was wetted with acetonitrile (0.5 mL). Triethylamine (1.05 eq, 69 μL, 0.49 mmol) and then phosphorus oxychloride (4 eq, 170 μL, 1.87 mmol) were added. The suspension was placed in a 100° C. oil bath. After 3.5 h, the solution was cooled to 23° C. and added dropwise to a solution of CH₃CN (5 mL), triethylamine (5 mL), and methanol (2.5 mL) cooled to 0° C. The solution was poured into water (25 mL) and extracted with EtOAc (3×30 mL). The organics were washed with water (2×50 mL) and brine (1×50 mL), then dried over MgSO₄, filtered and concentrated in vacuo to afford ethyl 4-chloroimidazo[1,5-a]quinoxaline-6-carboxylate (127 mg, 99%) as a dark red foamy solid. The crude solid was used in the next reaction without further purification. LCMS (ES): >85% pure, m/z 276 [M+1]⁺.

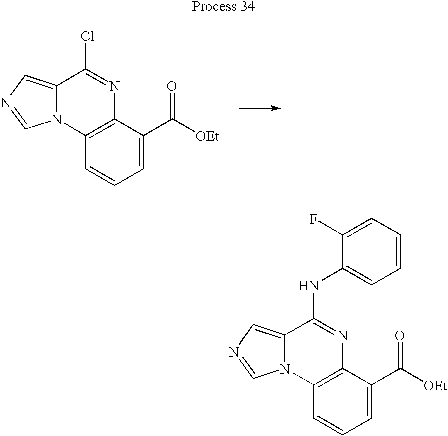

Ethyl 4-chloroimidazo[1,5-a]quinoxaline-6-carboxylate (1 eq, 127 mg, 0.46 mmol) was dissolved in NMP (620 μL). 2-fluoroaniline (2 eq, 90 μL, 0.93 mmol) was added and the reaction was sealed and heated under microwave irradiation (140° C.) for 10 min. The reaction was diluted with water (25 mL) and extracted with EtOAc (3×25 mL). The organics were washed with brine (1×75 mL), dried over MgSO₄, filtered and concentrated in vacuo to a brown oil. Purification via flash column chromatography (50%-100% EtOAc/hexanes) provided ethyl 4-(2-fluorophenylamino)imidazo[1,5-a]quinoxaline-6-carboxylate (84 mg, 50% over two steps) as a brown solid. LCMS (ES): >90% pure, m/z 351 [M+1]⁺.

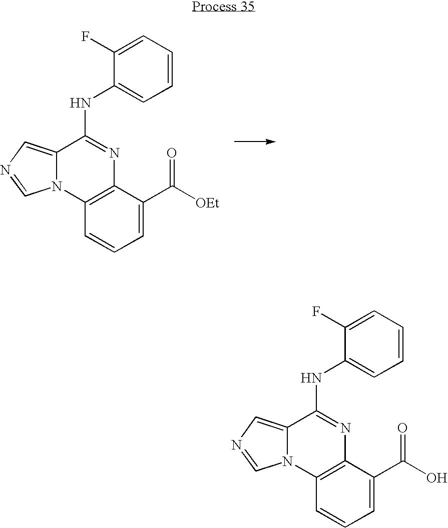

Ethyl 4-(2-fluorophenylamino)imidazo[1,5-a]quinoxaline-6-carboxylate (84 mg, 0.24 mmol) was dissolved in ethanol (1.2 mL, denatured with 5% methanol and 5% IPA). 6M NaOH (0.3 mL) was added and the reaction was placed in an 80° C. oil bath. After 45 min, the solution was added to water (25 mL). The solution was cooled in an ice bath and 6M HCl was added dropwise until pH=2. The filtrate was extracted with EtOAc (3×25 mL). The organics were washed with brine (1×50 mL), dried over MgSO₄, filtered, and concentrated in vacuo to yield 4-2-fluorophenylamino)imidazo[1,5-a]quinoxaline-6-carboxylic acid as a maize colored solid (46 mg, 60%). LCMS (ES): >95% pure, m/z 323 [M+1]⁺.

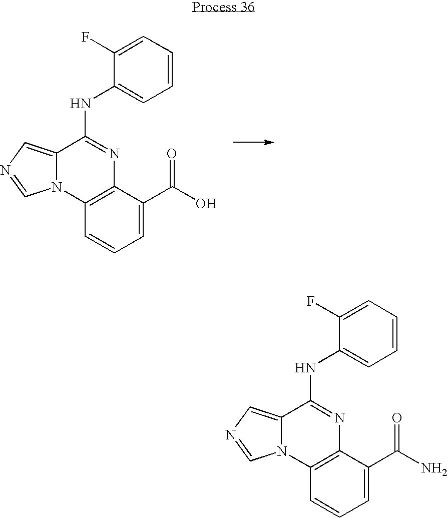

4-oxo-4,5-dihydropyrrolo[1,2-a]quinoxaline-6-carboxylic acid (1 eq, 29 mg, 0.09 mmol) was suspended in 1,4-dioxane (1 mL). Ammonium chloride (8 eq, 38 mg, 0.72 mmol), EDCI.HCl (2 eq, 34 mg, 0.18 mmol), and HOBt (2 eq, 24 mg, 0.18 mmol) were added in one portion in sequential order. DIEA (8 eq, 123 μL, 22.9 mmol) was added rapidly dropwise. The mixture was stirred for 2 min, then placed in an 80° C. oil bath. After 1 h, the solution was cooled to 23° C. and then ammonium chloride (8 eq, 38 mg, 0.72 mmol), EDCI.HCl (2 eq, 34 mg, 0.18 mmol), and HOBt (2 eq, 24 mg, 0.18 mmol) were added in one portion in sequential order. DIEA (8 eq, 123 μL, 22.9 mmol) was added rapidly dropwise and the solution was placed back into an 80° C. oil bath. After 1 h, the solution was cooled to 23° C. and poured into water (10 mL). The solution was extracted with EtOAc (3×25 mL) and the organics were washed with brine (1×50 mL), dried over MgSO₄, filtered, and concentrated in vacuo. The residue was purified via flash column chromatography to yield 4-2-fluorophenylamino)imidazo[1,5-a]quinoxaline-6-carboxamide (15 mg, 52%) as a tan/orange colored solid. LCMS (ES): >95% pure, m/z 322 [M+1]⁺.

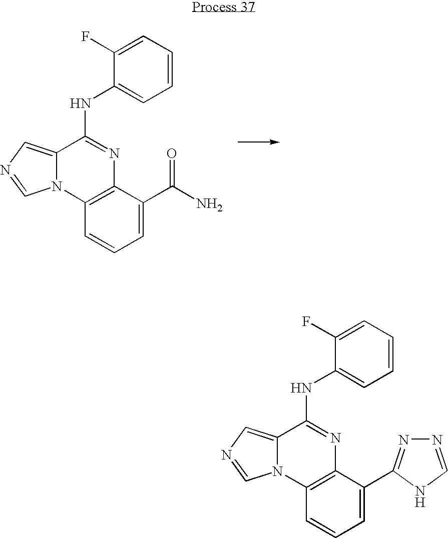

4-oxo-4,5-dihydropyrrolo[1,2-a]quinoxaline-6-carboxamide (14 mg, 0.04 mmol) was suspended in dimethylformamide dimethylacetal (500 μL). The suspension was placed in an 80° C. oil bath. After 1 h, more dimethylformamide dimethylacetal (500 μL) was added and the reaction was placed back into an 80° C. oil bath. After an additional hour, the solution was concentrated in vacuo and used crude for the next reaction. The crude solid was dissolved in glacial acetic acid (1 mL). NH₂NH₂.H₂O (200 μL) was added dropwise. The reaction was placed in an 80° C. oil bath for 1 h. The solution was cooled to 23° C., then poured into water (10 mL). The solution was extracted with EtOAc (3×10 mL) and the organics were washed with brine (1×25 mL), dried over MgSO₄, filtered, and concentrated in vacuo. The residue was purified via flash column chromatography (2.5-5% MeOH/CH₂Cl₂) to afford N-(2-fluorophenyl)-6-(4H-1,2,4-triazol-3-yl)imidazo[1,5-a]quinoxaline-4-amine (4 mg, 26% over two steps). LCMS (ES): >90% pure, m/z 346 [M+1]⁺.

### Example 7

### Processes for Synthesis of Pyrazole Compounds

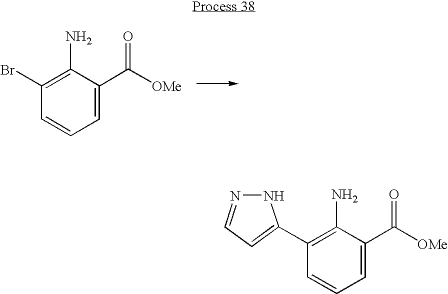

Methyl 2-amino-3-bromobenzoate (1 eq, 50 mg, 0.22 mmol) and 1H-pyrazole-5-boronic acid pinacol ester (1.3 eq, 55 mg, 0.28 mmol) were dissolved in DMF (5.4 mL). 2M Na₂CO₃ (2 eq, 217 μL, 0.43 mmol) was added, and the solution was degassed for 10 min with a stream of nitrogen. PdCl₂dppf.CH₂Cl₂ (0.2 eq, 35 mg, 0.04 mmol) was added and the reaction sealed and irradiated at 100° C. for 45 min in a microwave. The solution was poured into water (50 mL) and a black solid was filtered off. The crude solid was purified via flash column chromatography (30-40% EtOAc/hexanes) to furnish methyl 2-amino-3-(1H-pyrazol-5-yl)benzoate (15 mg, 32% as a yellow solid. ¹H NMR (CDCl₃, 400 MHz) δ: 7.91 (dd, 1H, J=8.0, 2.0 Hz), 7.86 (bs, 2H), 7.68 (dd, 1H, J=7.2, 1.2 Hz), 7.63 (d, 1H, J=2.8 Hz), 6.63-6.68 (m, 2H) 3.95 (s, 3H). LCMS (ES): >90% pure, m/z 218 [M+1]⁺.

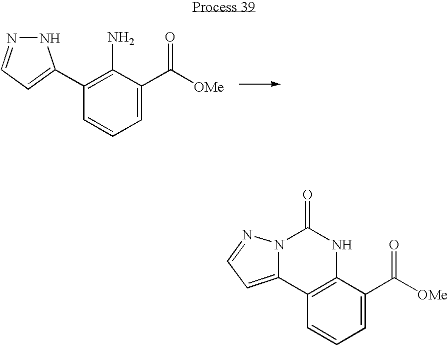

Methyl 2-amino-3-(1H-pyrazol-5-yl)benzoate (1.0 eq, 118 mg, 0.54 mmol) and triphosgene (3 eq, 484 mg, 1.63 mmol) were dissolved in anhydrous toluene (3.6 mL), then placed in a 120° C. oil bath. After 2.25 h, the solution was cooled to 23° C. and then poured into cold hexanes (30 mL). The solution stirred for 5 min and then the brown/gray precipitate, methyl 5-oxo-5,6-dihydropyrrolo[1,5-c]quinazoline-7-carboxylate (78 mg, 59%), was filtered off. LCMS (ES): >95% pure, m/z 244 [M+1]+.

### Biodata Test Methods

### Example 8

### CK2 Assay Method

Modulatory activity of compounds described herein was assessed in vitro in cell-free CK2 assays by the following method.

Test compounds in aqueous solution were added at a volume of 10 microliters, to a reaction mixture comprising 10 microliters Assay Dilution Buffer (ADB; 20 mM MOPS, pH 7.2, 25 mM beta-glycerolphosphate, 5 mM EGTA, 1 mM sodium orthovanadate and 1 mM dithiothreitol), 10 microliters of substrate peptide (RRRDDDSDDD, dissolved in ADB at a concentration of 1 mM), 10 microliters of recombinant human CK2 (25 ng dissolved in ADB; Upstate). Reactions were initiated by the addition of 10 microliters of ATP Solution (90% 75 mM MgCl₂, 75 micromolar ATP dissolved in ADB; 10% [γ-³³P]ATP (stock 1 mCi/100 μl; 3000 Ci/mmol (Perkin Elmer) and maintained for 10 minutes at 30 degrees C. The reactions were quenched with 100 microliters of 0.75% phosphoric acid, then transferred to and filtered through a phosphocellulose filter plate (Millipore). After washing each well 5 times with 0.75% phosphoric acid, the plate was dried under vacuum for 5 min and, following the addition of 15 ul of scintilation fluid to each well, the residual radioactivity was measured using a Wallac luminescence counter.

### Example 9

### PIM-1 Assay Method

The following procedure was used to assay the PIM-1 kinase activity of compounds of the invention. Other methods for assaying PIM-1 and other PIM kinases, as well as methods to assay for activity against various kinases known in the art.

In a final reaction volume of 50 ul, recombinant PIM-1 (1 ng) was incubated with 12 mM MOPS pH 7.0, 0.4 mM EDTA, glycerol 1%, brij 35 0.002%, 2-mercaptoethanol 0.02%, BSA 0.2 mg/ml, 100 uM KKRNRTLTK, 10 mM MgAcetate, 15 uM ATP, [γ-³³P-ATP] (specific activity approx. 500 cpm/pmol), DMSO 4% and test inhibitor compound at the required concentration. The reaction was initiated by the addition of the Magnesium ATP mixture. After 40 min incubation at 23° C., the reactions were quenched by the addition of 100 ul 0.75% Phosphoric acid, and the labeled peptide collected by filtration through a phosphocellulose filter plate. The plate was washed 4 times with 0.075% phosphoric acid (100 ul per well) and then, after the addition of scintillation fluid (20 ul per well), the counts were measured by a scintillation counter.

### Example 10

### PIM-2 Assay Method

Test compounds dissolved and diluted in DMSO (2 μl) were added to a reaction mixture comprising 10 μl of 5× Reaction Buffer (40 mM MOPS pH 7.0, 5 mM EDTA), 10 μl of recombinant human PIM2 solution (4 ng PIM-2 dissolved in dilution buffer (20 mM MOPS pH 7.0; EDTA 1 mM; 5% Glycerol; 0.01% Brij 35; 0.1%; 0.1% 2-mercaptoethanol; 1 mg/ml BSA)) and 8 ul of water. Reactions were initiated by the addition of 10 ul of ATP Solution (49% (15 mM MgCl₂; 75 uM ATP) 1% ([γ-33P]ATP: Stock 1 mCi/100 μl; 3000 Ci/mmol (Perkin Elmer)) and 10 ul of substrate peptide solution (RSRSSYPAGT, dissolved in water at a concentration of 1 mM), Reactions were maintained for 10 min at 30° C. The reactions were quenched with 100 ul of 0.75% Phosphoric acid, then transferred to and filtered through a Phosphocellulose filter plate (Millipore, MSPH-N6B-50). After washing each well 4 times with 0.75% Phosphoric acid, scintillation fluid (20 uL) was added to each well and the residual radioactivity was measured using a Wallac luminescence counter.

### Example 11

### Cell Proliferation Modulatory Activity

A representative cell-proliferation assay protocol using Alamar Blue dye (stored at 4° C., use 20 ul per well) is described hereafter.

### 96-Well Plate Setup and Compound Treatment

a. Split and trypsinize cells.

b. Count cells using hemocytometer.

c. Plate 4,000-5,000 cells per well in 100 μl of medium and seed into a 96-well plate according to the following plate layout. Add cell culture medium only to wells B10 to B12. Wells B1 to B9 have cells but no compound added.

| | 1 2 3 | 4 5 6 | 7 8 9 | 10 11 12 | |
|---|---|---|---|---|---|
| A | EMPTY | | | | |
| B | NO COMPOUND ADDED | | | Medium Only | |
| C | 10 nM | 100 nM | 1 uM | 10 uM | Control |
| D | 10 nM | 100 nM | 1 uM | 10 uM | Comp1 |
| E | 10 nM | 100 nM | 1 uM | 10 uM | Comp2 |
| F | 10 nM | 100 nM | 1 uM | 10 uM | Comp3 |
| G | 10 nM | 100 nM | 1 uM | 10 uM | Comp4 |

| | |
|---|---|
| H | EMPTY |

d. Add 100 μl of 2× drug dilution to each well in a concentration shown in the plate layout above. At the same time, add 100 μl of media into the control wells (wells B10 to B12). Total volume is 200 μl/well.

e. Incubate four (4) days at 37° C., 5% CO₂ in a humidified incubator.

f. Add 20 μl Alamar Blue reagent to each well.

g. Incubate for four (4) hours at 37° C., 5% CO₂ in a humidified incubator.

h. Record fluorescence at an excitation wavelength of 544 nm and emission wavelength of 590 nm using a microplate reader.

In the assays, cells are cultured with a test compound for approximately four days, the dye is then added to the cells and fluorescence of non-reduced dye is detected after approximately four hours. Different types of cells can be utilized in the assays (e.g., HCT-116 human colorectal carcinoma cells, PC-3 human prostatic cancer cells, MDA-MB231 human breast cancer cells, K-562 human chronic myelogenous leukemia (CML) cells, MiaPaca human pancreatic carcinoma cells, MV-4 human acute myeloid leukemia cells, and BxPC3 human pancreatic adenocarcinoma cells).

Biological activities for various compounds are summarized in the following tables (Table 4 and Table 5).

TABLE 4
Biological data for representative compounds

| | | PIM1: | | MV- | MDAM | Mia- | |
|---|---|---|---|---|---|---|---|
| | PIM1: | IC₅₀ | K-562 | 4-11 | B231 | PaCa | PC3 |
| | % inh | (15 μM | IC₅₀ | IC₅₀ | IC₅₀ | IC₅₀ | IC₅₀ |
| Structure | 500 nM | ATP) | (μM) | (μM) | (μM) | (μM) | (μM) |
| 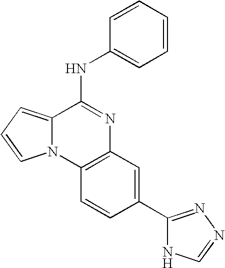 | −24.476 | | | | | | |
| 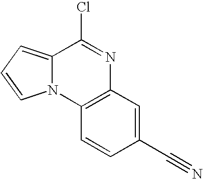 | −35.427 | | | | | | |
| 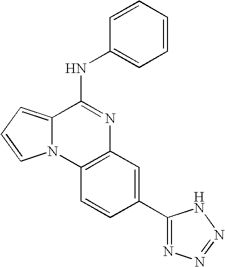 | 75.869 | 0.164 | >10 | >10 | | | |
| 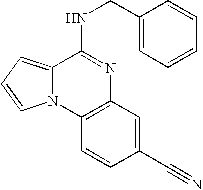 | −34.986 | | | | | | |
| 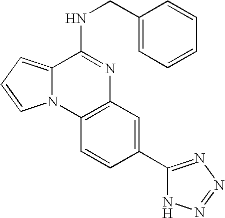 | 14.381 | | | | | | |
| 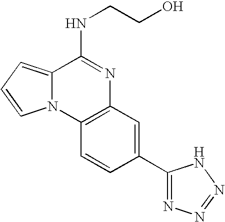 | 67.872 | 0.273 | >10 | >10 | | | |
| 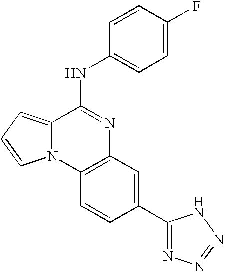 | 73.165 | 0.365 | >10 | >10 | | | |
| 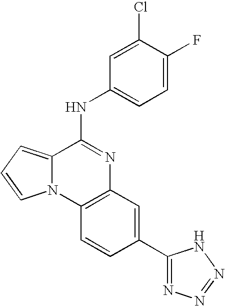 | 50.467 | 0.504 | >10 | 1.21 | >10 | >10 | |
| 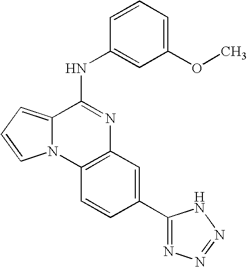 | 15.033 | | | | | | |
| 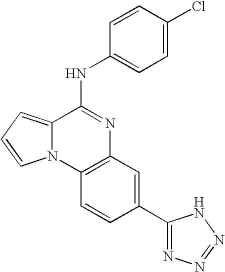 | 57.112 | 0.576 | >10 | >10 | >10 | >10 | |
| 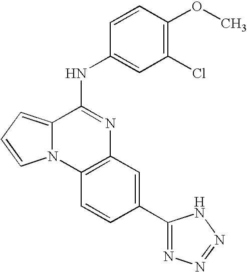 | 19.406 | | | | | | |
| 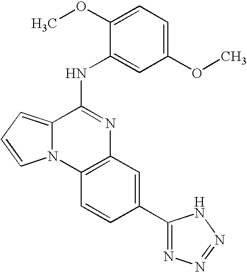 | 10.938 | | | | | | |
| 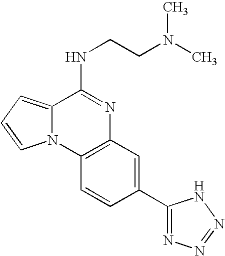 | 63.509 | 0.486 | >10 | >10 | | | |
| 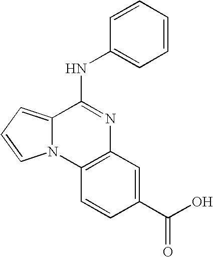 | 73.079 | 0.13 | >10 | 0.483 | | | |
| 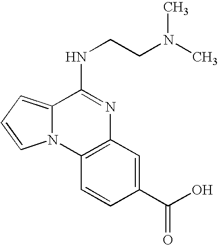 | 76.454 | 0.173 | >10 | 0.688 | | | |
| 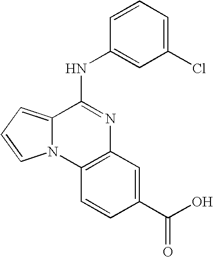 | 95.395 | 0.095 | >10 | 2.925 | | 8.918 | >10 |
| 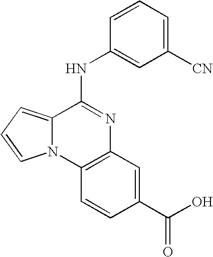 | 92.304 | 0.115 | >10 | >10 | | >10 | >10 |
| 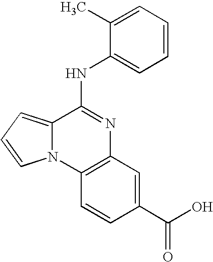 | 43.795 | | >10 | >10 | | >10 | >10 |
| 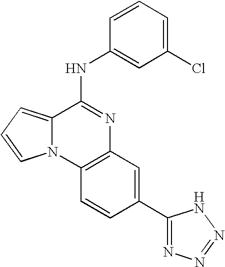 | 77.417 | 0.15 | >10 | 4.976 | | 9.368 | >10 |
| 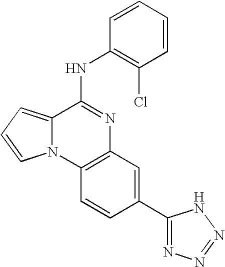 | 59.256 | 0.466 | >10 | >10 | | >10 | >10 |
| 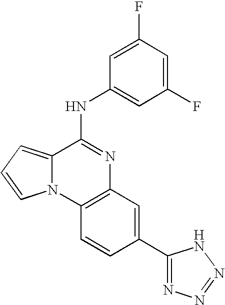 | 63.848 | 0.514 | >10 | 7.769 | | >10 | >10 |
| 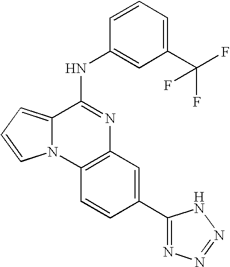 | 45.182 | | >10 | 6.585 | | >10 | >10 |
| 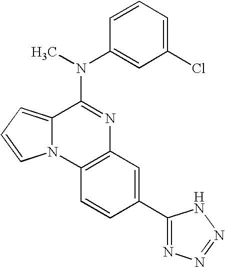 | 6.586 | | >10 | >10 | | >10 | >10 |
| 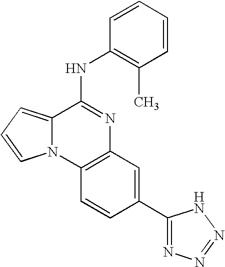 | 52.353 | 0.676 | >10 | >10 | | >10 | >10 |
| 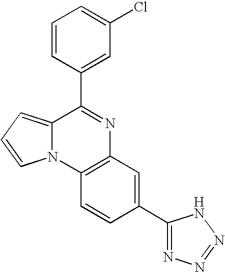 | 25.043 | | >10 | >10 | | >10 | >10 |
| 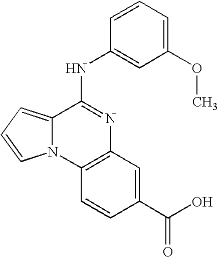 | 78.955 | 0.354 | >10 | >10 | | >10 | >10 |
| 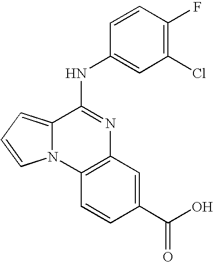 | 91.449 | 0.071 | 10.709 | 6.909 | 11.994 | >10 | >10 |
| 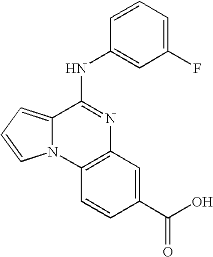 | 78.107 | 0.156 | >10 | >10 | | >10 | >10 |
| 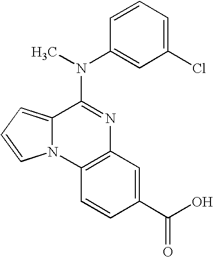 | 49.713 | | >10 | >10 | | >10 | >10 |
| 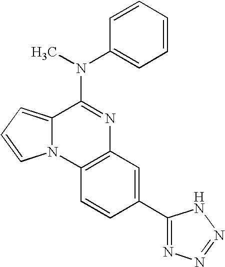 | 15.335 | | >10 | >10 | | >10 | >10 |
| 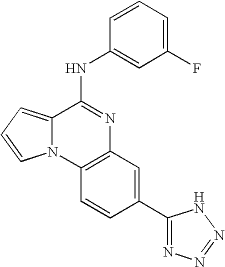 | 70.039 | 0.29 | >10 | >10 | | >10 | >10 |
| 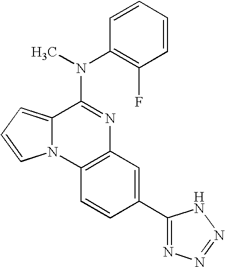 | 51.367 | 0.774 | >10 | >10 | | >10 | >10 |
| 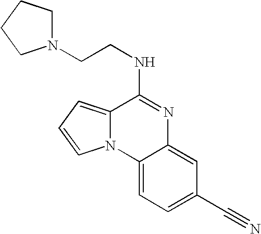 | 10.317 | | 5.517 | 3.001 | | 1.244 | 9.651 |
| 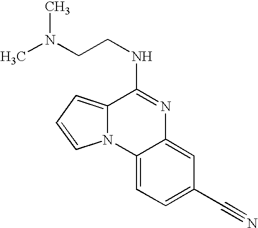 | 20.116 | | 5.239 | 2.731 | | 1.294 | 8.313 |
| 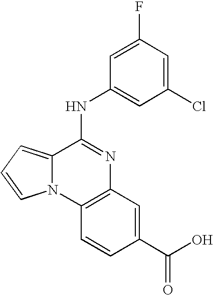 | 83.921 | 0.119 | >10 | 8.551 | | >10 | >10 |
| 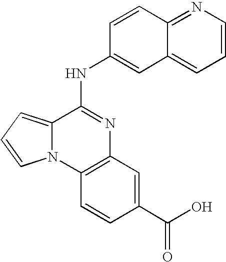 | 43.221 | 0.239 | >10 | >10 | | >10 | |
| 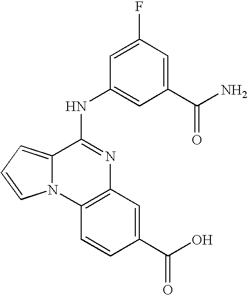 | 69.332 | 0.229 | >10 | >10 | | >10 | |
| 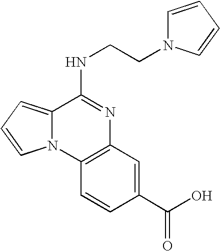 | 50.106 | 0.554 | >10 | >10 | | >10 | >10 |
| 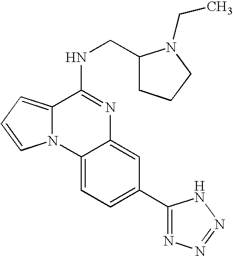 | 81.535 | 0.097 | >10 | >10 | | | |
| 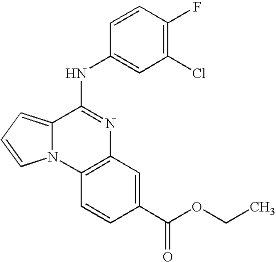 | −76.203 | | | | | | |
| 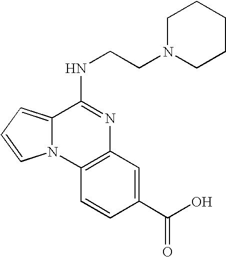 | 98.785 | 0.372 | >10 | >10 | | | |
| 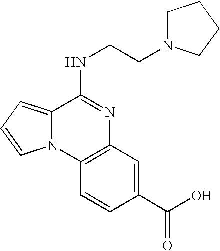 | 72.544 | 0.504 | >10 | >10 | | | |
| 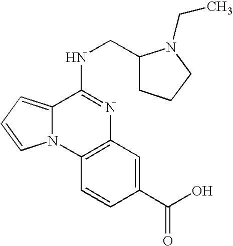 | 89.671 | 0.2 | >10 | >10 | | | |
| 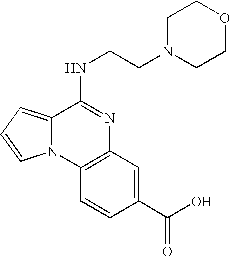 | 18.886 | | | | | | |
| 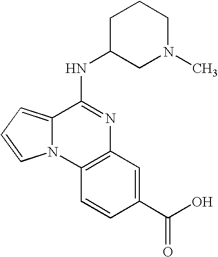 | 69.506 | 0.33 | >10 | >10 | | | |
| 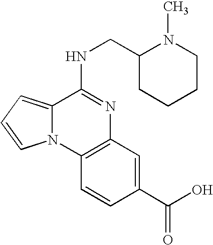 | | | | | | | |
| 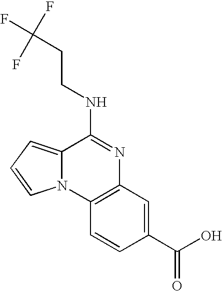 | 45.953 | | | | | | |
| 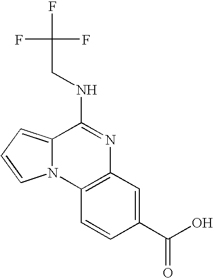 | 52.895 | 0.419 | >10 | >10 | >10 | >10 | >30 |
| 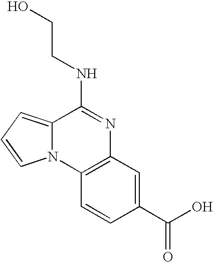 | 35.031 | | | | | | |
| 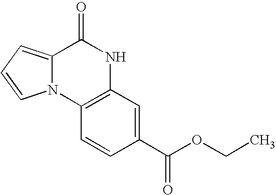 | 14.014 | | | | | | |
| 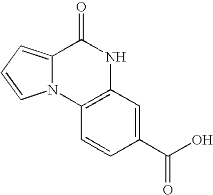 | 25.567 | | | | | | |
| 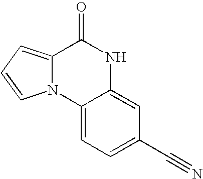 | −28.551 | | | | | | |
| 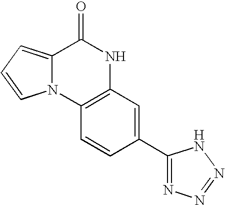 | 41.769 | | | | | | |
| 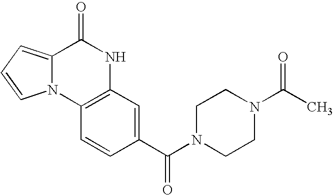 | −8.346 | | | | | | |
| 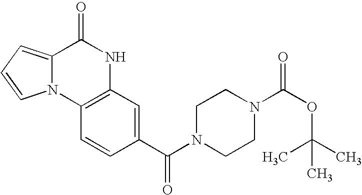 | −27.075 | | | | | | |
| 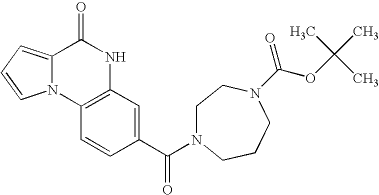 | −46.935 | | | | | | |
| 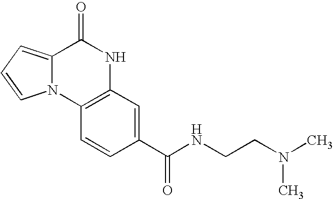 | −13.918 | | | | | | |
| 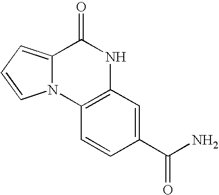 | −22.452 | | | | | | |
| 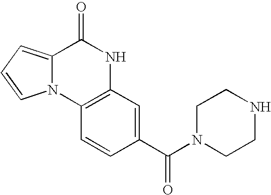 | −11.827 | | | | | | |
| 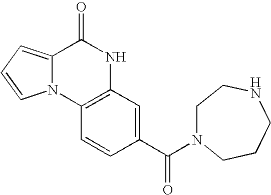 | −0.195 | | | | | | |
| 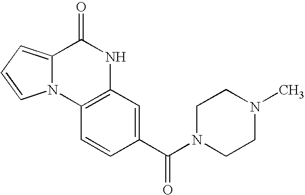 | −31.95 | | | | | | |

TABLE 5
Biological data for representative compounds

| | | | | | | MDA- | | SUM- | |
|---|---|---|---|---|---|---|---|---|---|
| | CK2 | PIM1 | PIM2 | FLT3 | BxPC3 | MB45 | MV4-11 | 149PT | K562 |
| | IC₅₀ | IC₅₀ | IC₅₀ | IC₅₀ | IC₅₀ | IC₅₀ | IC₅₀ | IC₅₀ | IC₅₀ |
| Structure | (μM) | (μM) | (μM) | (μM) | (μM) | (μM) | (μM) | (μM) | (μM) |
| 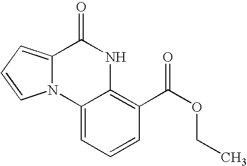 | >5.0 | >2.5 | >2.5 | | | | | | |
| 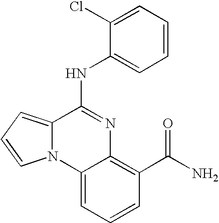 | <1.0 | <0.1 | <1.0 | | | | | | |
| 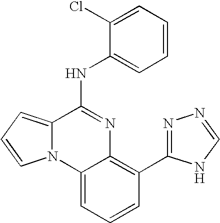 | <0.1 | <0.1 | <0.1 | >10 | <10.0 | <5.0 | <5.0 | <5.0 | >10 |
| 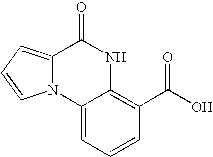 | <5.0 | >2.5 | >2.5 | | | | | | |
| 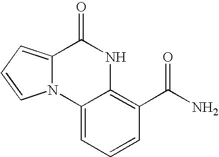 | >5.0 | >2.5 | >2.5 | | | | | | |
| 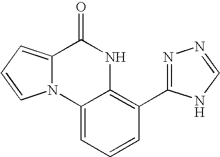 | <5.0 | >2.5 | >2.5 | | | | | | |
| 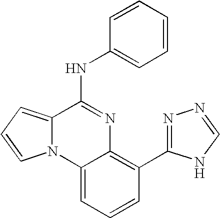 | <0.1 | <0.1 | <0.1 | | >30 | 15.1 | <5.0 | >30 | >30 |
| 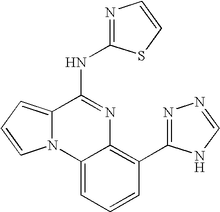 | <5.0 | <5.0 | <5.0 | | | | | | |
| 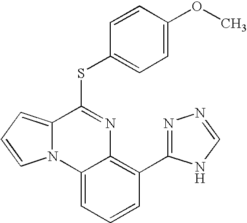 | <0.1 | <0.1 | <5.0 | | | | | | |
| 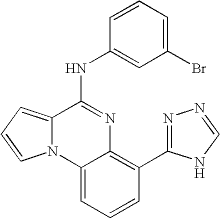 | <0.1 | <0.1 | <0.1 | | | | | | |
| 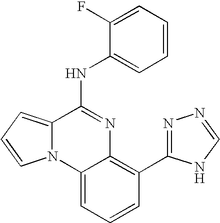 | <0.1 | <0.1 | <0.1 | | >30 | 28.4 | <5.0 | 22.8 | <10.0 |
| 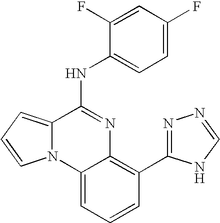 | <0.1 | <0.1 | <0.1 | | 10.2 | 15.7 | <5.0 | 23.3 | <10.0 |
| 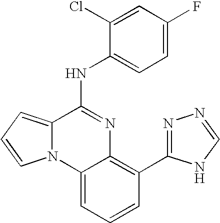 | <1.0 | <0.1 | <0.1 | | | | | | |
| 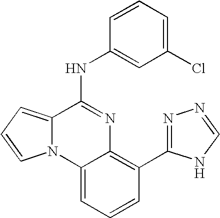 | <0.1 | <0.1 | <0.1 | | 30 | 16 | <5.0 | 21 | >10 |
| 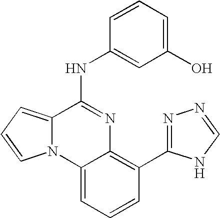 | <1.0 | <0.1 | <0.1 | | 30 | 28 | <5.0 | >30 | >10 |
| 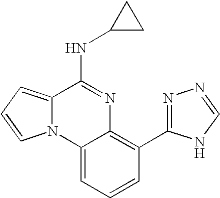 | <1.0 | <0.1 | <1.0 | | | | | | |
| 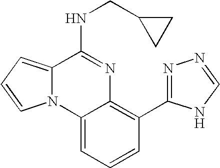 | <5.0 | <0.1 | <1.0 | | | | | | |
| 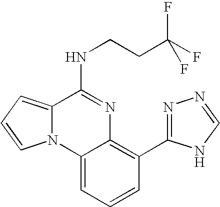 | <5.0 | <0.1 | <1.0 | | | | | | |
| 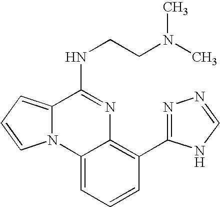 | >5.0 | >2.5 | >2.5 | | | | | | |
| 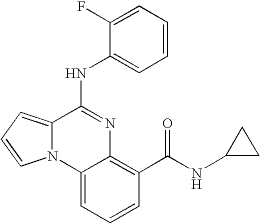 | <1.0 | <0.1 | <1.0 | | >30 | 12.0 | <10.0 | <10.0 | >10 |
| 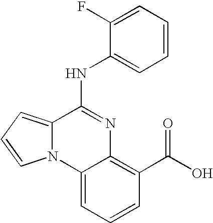 | <1.0 | <1.0 | <1.0 | | | | | | |
| 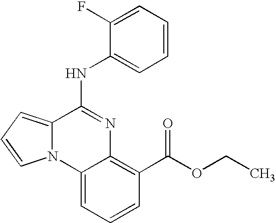 | >5.0 | >2.5 | >2.5 | | | | | | |
| 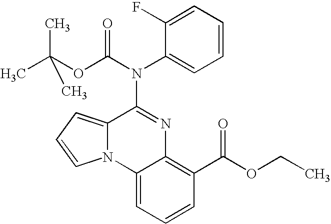 | >5.0 | >2.5 | >2.5 | | | | | | |
| 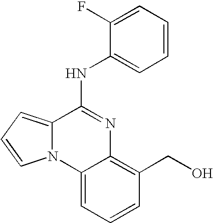 | >5.0 | >2.50 | <5.0 | | | | | | |
| 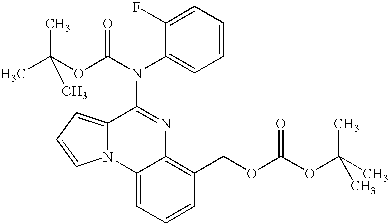 | >5.0 | >2.5 | >2.5 | | | | | | |
| 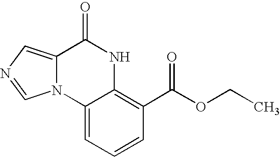 | <5.0 | | >2.5 | | | | | | |
| 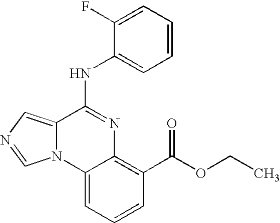 | <5.0 | | >2.5 | | | | | | |
| 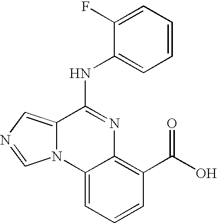 | <1.0 | | >2.50 | | | | | | |
| 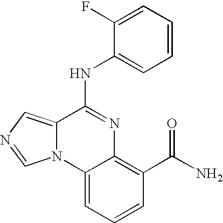 | <0.1 | | <0.1 | | | | | | |
| 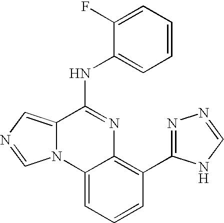 | <0.1 | | <0.1 | | 15.1 | <5.0 | <1.0 | <10.0 | <10.0 |
| 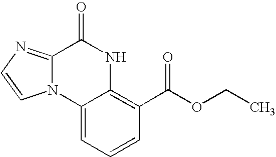 | >5.0 | | >2.5 | | | | | | |

### Example 12

### Representative Embodiments of the Invention

A1. A compound having a structure of Formula I:

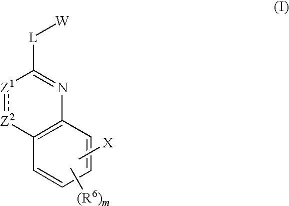

wherein:
Z¹ and Z² are independently selected from N, NR¹, C═V, and CR², provided Z¹ and Z² are not both NR¹;
   where R¹ and R² are independently selected from H, optionally substituted amino, optionally substituted alkoxy, optionally substituted alkyl, optionally substituted alkenyl, optionally substituted aryl, optionally substituted heteroaryl, optionally substituted heteroalkyl, optionally substituted heteroalkenyl, optionally substituted cycloalkyl, and optionally substituted heterocycloalkyl;

C═V is C═CR¹R⁵ or C═NR¹;
and two R¹ groups, or R¹ and R² if both are present, can be taken together to form a 5-6 membered optionally substituted heterocyclic ring;

L is a linker selected from a bond, NR³, O, S, CR³R⁴, CR³R⁴—NR³, CR³R⁴—O—, and CR³R⁴—S;
where each R³, R⁴, R⁵, and R⁶ is independently H, or an optionally substituted member selected from the group consisting of C1-C8 alkyl, C2-C8 heteroalkyl, C2-C8 alkenyl, C2-C8 heteroalkenyl, C2-C8 alkynyl, C2-C8 heteroalkynyl, C1-C8 acyl, C2-C8 heteroacyl, C6-C10 aryl, C5-C12 heteroaryl, C7-C12 arylalkyl, and C6-C12 heteroarylalkyl group,
   or halo, OR, NR₂, NROR, NRNR₂, SR, SOR, SO₂R, SO₂NR₂, NRSO₂R, NRCONR₂, NRCSNR₂, NRC(═NR)NR₂, NRCOOR, NRCOR, CN, COOR, CONR₂, OOCR, COR, or NO₂,
   wherein each R is independently H or C1-C8 alkyl, C2-C8 heteroalkyl, C2-C8 alkenyl, C2-C8 heteroalkenyl, C2-C8 alkynyl, C2-C8 heteroalkynyl, C1-C8 acyl, C2-C8 heteroacyl, C6-C10 aryl, C5-C10 heteroaryl, C7-C12 arylalkyl, or C6-C12 heteroarylalkyl,
   and wherein two R on the same atom or on adjacent atoms can be linked to form a 3-8 membered ring, optionally containing one or more N, O or S;
      and each R group, and each ring formed by linking two R groups together, is optionally substituted with one or more substituents selected from halo, ═O, ═N—CN, ═N—OR′, ═NR′, OR′, NR′₂, SR′, SO₂R′, SO₂NR′₂, NR′SO₂R′, NR′CONR′₂, NR′CSNR′₂, NR′C(═NR′)NR′₂, NR′COOR′, NR′COR′, CN, COOR′, CONR′₂, OOCR′, COR′, and NO₂,
      wherein each R′ is independently H, C1-C6 alkyl, C2-C6 heteroalkyl, C1-C6 acyl, C2-C6 heteroacyl, C6-C10 aryl, C5-C10 heteroaryl, C7-12 arylalkyl, or C6-12 heteroarylalkyl, each of which is optionally substituted with one or more groups selected from halo, C1-C4 alkyl, C1-C4 heteroalkyl, C1-C6 acyl, C1-C6 heteroacyl, hydroxy, amino, and ═O;
         and wherein two R′ on the same atom or on adjacent atoms can be linked to form a 3-7 membered ring optionally containing up to three heteroatoms selected from N, O and S;
   and R³ and R⁴, when on the same atom or on adjacent connected atoms, can optionally be linked together to form a 3-8 membered cycloalkyl or heterocycloalkyl, which is optionally substituted;
W is alkyl, heteroalkyl, aryl, heteroaryl, cycloalkyl, or heterocyclyl, each of which can be optionally substituted;
X is a polar substituent;
and m is 0-2;

or a pharmaceutically acceptable salt thereof.

A2. The compound of embodiment A1, wherein Z¹ is CR² and Z² is NR¹.

A3. The compound of embodiment A2, wherein R² and R¹ are linked together to form a 5-6 membered ring.

A4. The compound of embodiment A1, wherein Z¹ and Z² are both CR², or Z¹ and Z² are both N.

A5. The compound of embodiment A1, wherein Z² is N.

A6. The compound of embodiment A5, wherein Z³ is also N.

A7. The compound of embodiment A5, wherein Z⁴ is also N.

A8. The compound of embodiment A5, wherein Z⁵ is also N.

A9. The compound of embodiment A5, wherein Z³ and Z⁴ are also N.

A10. The compound of embodiment A5, wherein Z³, Z⁴, Z⁵ are each CR⁵.

A11. The compound of embodiment A1, wherein the compound has the formula Ia:

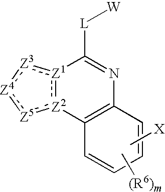

where one of Z¹ and Z² is N, and the other of these is C;
and Z³-Z⁵ are each independently CR⁵ or N,

and the dotted line bonds in the ring containing Z¹-Z⁵ indicate each of these bonds can be a single bond, double bond, or aromatic bond.

A12. The compound of embodiment A11, wherein the dotted line bonds in the ring containing Z¹-Z⁵ is an aromatic ring.

A13. The compound of embodiment A11 or A12, wherein Z¹ is C and Z² is N.

A14. The compound of embodiment A11 or A12, wherein Z¹ is N and Z² is C.

A15. The compound of embodiment A13 or A14, wherein at least one of Z³-Z⁵ is N.

A16. The compound of embodiment A13, A14 or A15, wherein at least two of Z³-Z⁵ are N.

A17. The compound of any one of embodiments A1-A16, wherein L is NH or NMe.

A18. The compound of any one of embodiments A1-A17, wherein W is selected from optionally substituted alkyl, optionally substituted aryl, optionally substituted cycloalkyl, and optionally substituted heterocyclyl.

A19. The compound of any one of embodiments A1-A18, wherein W is optionally substituted phenyl, optionally substituted heterocyclyl, or C1-C4 alkyl substituted with at least one member selected from the group consisting of optionally substituted phenyl, optionally substituted heteroalkyl, optionally substituted heteroaryl, halo, and —NR″₂,

where each R″ is independently H or optionally substituted C1-C6 alkyl;

or two R″ taken together with the N to which they are attached can be linked together to form an optionally substituted 3-8 membered ring, which can contain another heteroatom selected from N, O and S as a ring member, and can be saturated, unsaturated or aromatic.

A20. The compound of embodiment A19, wherein W comprises at least one group of the formula —(CH₂)ₚ—NR^{x}₂,

where p is 1-4,

R^{x} is independently at each occurrence H or optionally substituted alkyl;
or two R^{x} taken together with the N to which they are attached can be linked together to form an optionally substituted 3-8 membered ring, which can contain another heteroatom selected from N, O and S as a ring member, and can be saturated, unsaturated or aromatic.

A21. The compound of any one of embodiments A1-A17, wherein L is NH or NMe, and

W is optionally substituted phenyl, optionally substituted heterocyclyl, or C1-C4 alkyl substituted with at least one member selected from the group consisting of optionally substituted phenyl, optionally substituted heteroalkyl, optionally substituted heteroaryl, halo, and —NR″₂,

where each R″ is independently H or optionally substituted C1-C6 alkyl;
or two R″ taken together with the N to which they are attached can be linked together to form an optionally substituted 3-8 membered ring, which can contain another heteroatom selected from N, O and S as a ring member, and can be saturated, unsaturated or aromatic.

A22. The compound of any one of embodiments A1-A21, wherein X is selected from the group consisting of COOR⁹, C(O)NR⁹—OR⁹, triazole, tetrazole, CN, imidazole, carboxylate, a carboxylate bioisostere,

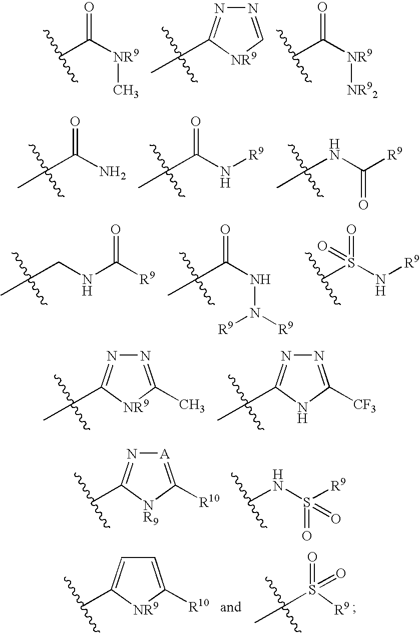

wherein each R⁹ is independently H or an optionally substituted member selected from the group consisting of alkyl, cycloalkyl, heterocyclyl, aryl, heteroaryl, arylalkyl, cycloalkylalkyl, heterocycloalkylalkyl, and heteroarylalkyl,
   and two R⁹ on the same or adjacent atoms can optionally be linked together to form an optionally substituted ring that can also contain an additional heteroatom selected from N, O and S as a ring member;
R¹⁰ is halo, CF₃, CN, SR, OR, NR₂, or R, where each R is independently H or optionally substituted C1-C6 alkyl, and two R on the same or adjacent atoms can optionally be linked together to form an optionally substituted ring that can also contain an additional heteroatom selected from N, O and S as a ring member;
and A is N or CR¹⁰.

A23. The compound of any one of embodiments A1-A22, wherein the polar substituent X is located at position 3 on the phenyl ring.

A24. The compound of any one of embodiments A1-A12, wherein the polar substituent X is located at position 4 on the phenyl ring.

A25. The compound of any one of embodiments A1-A16, wherein -L-W is selected from:

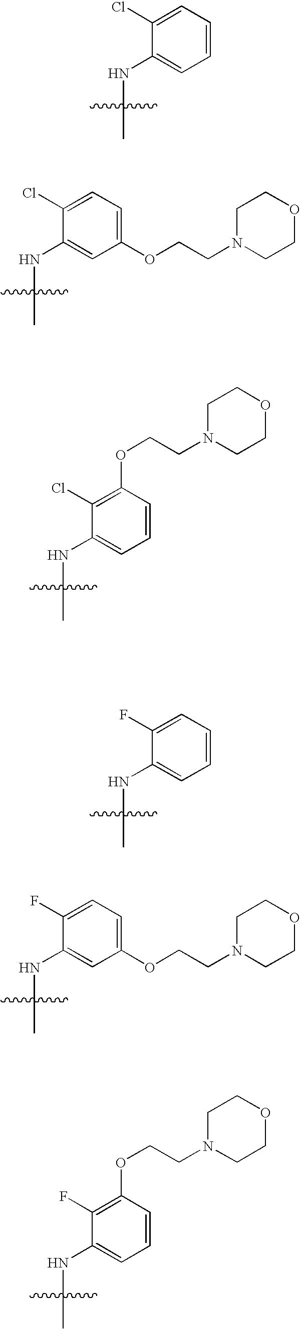

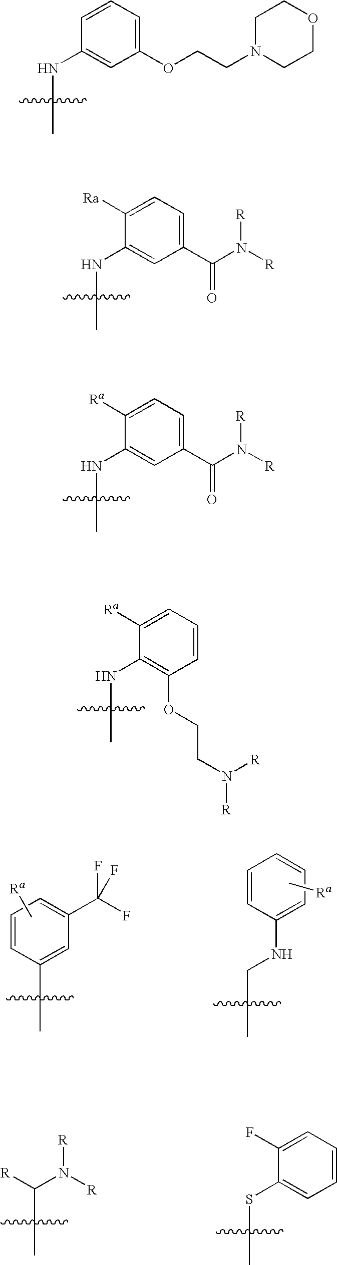

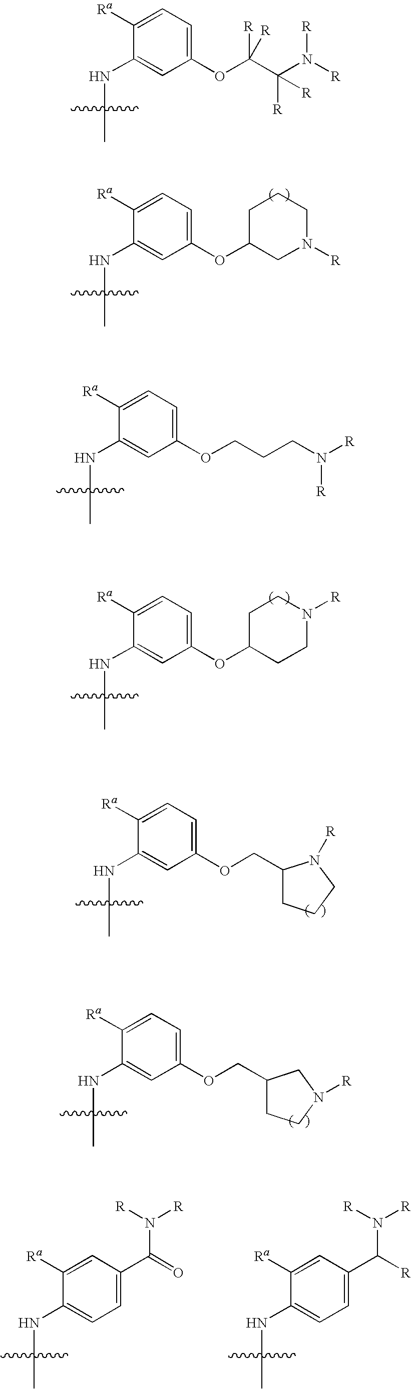

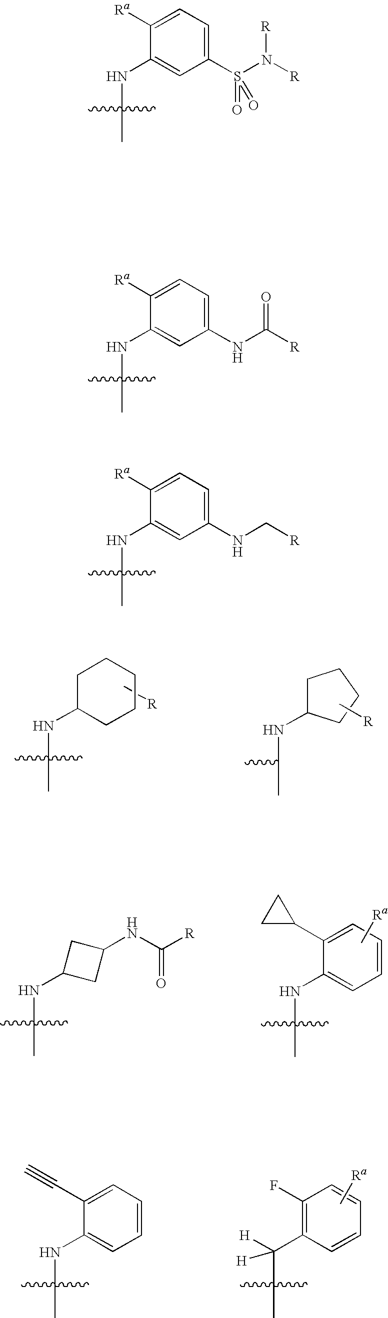

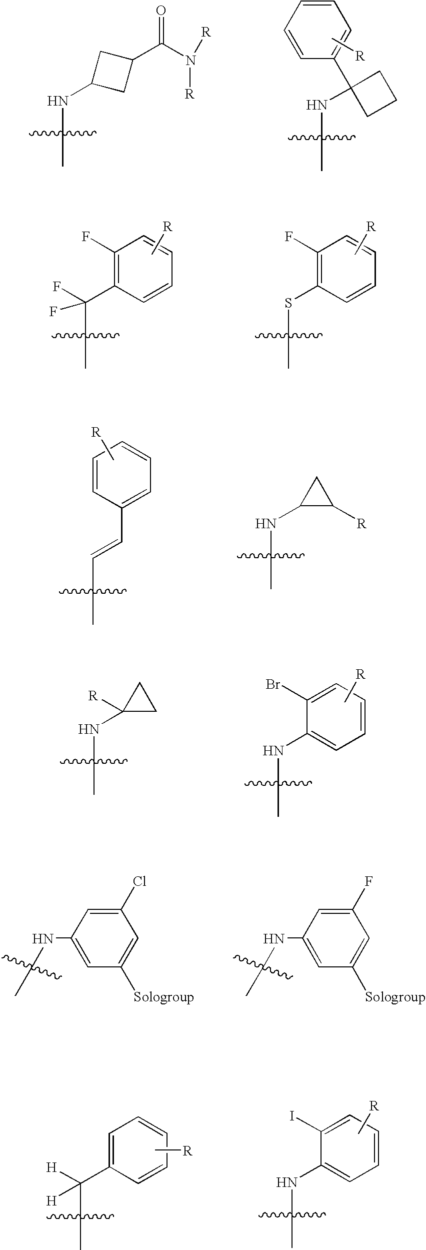

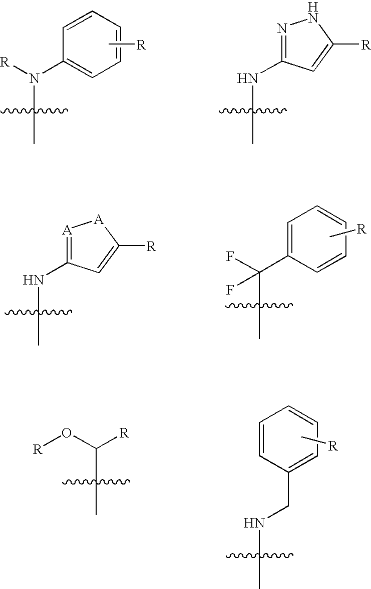

wherein each R¹ is H, Cl or F;
each R is independently selected from halo, C1-C4 alkyl, C1-C4 alkoxy, and C1-C4 haloalkyl,
   and two R groups on the same or adjacent connected atoms can optionally be linked together to form a 3-8 membered ring;
each A is N or CR;

and each Solgroup is a solubility-enhancing group.

B1. A compound of Formula II:

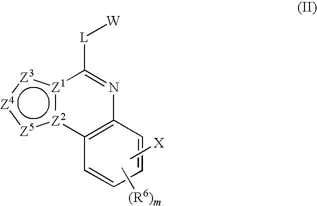

wherein Z¹ and Z² are each C, or one of Z¹ and Z² is N, the other of Z¹ and Z² is C;

Z³, Z⁴ and Z⁵ are independently selected from N, NR⁵, CR⁵ and O, provided not more than one of Z³-Z⁵ is O, and the ring containing Z³-Z⁵ is aromatic;

L is a linker selected from a bond, NR³, O, S, CR³R⁴, CR³R⁴—NR³, CR³R⁴—O—, and CR³R⁴—S;
where each R³, R⁴, R⁵, and R⁶ is independently H, or an optionally substituted member selected from the group consisting of C1-C8 alkyl, C2-C8 heteroalkyl, C2-C8 alkenyl, C2-C8 heteroalkenyl, C2-C8 alkynyl, C2-C8 heteroalkynyl, C1-C8 acyl, C2-C8 heteroacyl, C6-C10 aryl, C5-C12 heteroaryl, C7-C12 arylalkyl, and C6-C12 heteroarylalkyl group,
   or halo, OR, NR₂, NROR, NRNR₂, SR, SOR, SO₂R, SO₂NR₂, NRSO₂R, NRCONR₂, NRCSNR₂, NRC(═NR)NR₂, NRCOOR, NRCOR, CN, COOR, CONR₂, OOCR, COR, or NO₂,
   wherein each R is independently H or C1-C8 alkyl, C2-C8 heteroalkyl, C2-C8 alkenyl, C2-C8 heteroalkenyl, C2-C8 alkynyl, C2-C8 heteroalkynyl, C1-C8 acyl, C2-C8 heteroacyl, C6-C10 aryl, C5-C10 heteroaryl, C7-C12 arylalkyl, or C6-C12 heteroarylalkyl,
   and wherein two R on the same atom or on adjacent atoms can be linked to form a 3-8 membered ring, optionally containing one or more N, O or S;
      and each R group, and each ring formed by linking two R groups together, is optionally substituted with one or more substituents selected from halo, ═O, ═N—CN, ═NR′, OR′, NR′₂, SR′, SO₂R′, SO₂NR′₂, NR′SO₂R′, NR′CONR′₂, NR′CSNR′₂, NR′C(═NR′)NR′₂, NR′COOR′, NR′COR′, CN, COOR′, CONR′₂, OOCR′, COR′, and NO₂,
      wherein each R′ is independently H, C1-C6 alkyl, C2-C6 heteroalkyl, C1-C6 acyl, C2-C6 heteroacyl, C6-C10 aryl, C5-C10 heteroaryl, C7-12 arylalkyl, or C6-12 heteroarylalkyl, each of which is optionally substituted with one or more groups selected from halo, C1-C4 alkyl, C1-C4 heteroalkyl, C1-C6 acyl, C1-C6 heteroacyl, hydroxy, amino, and ═O;
         and wherein two R′ on the same atom or on adjacent atoms can be linked to form a 3-7 membered ring optionally containing up to three heteroatoms selected from N, O and S;
   and R³ and R⁴, when on the same atom or on adjacent connected atoms, can optionally be linked together to form a 3-8 membered cycloalkyl or heterocycloalkyl, which is optionally substituted;
W is alkyl, heteroalkyl, aryl, heteroaryl, cycloalkyl, or heterocyclyl, each of which can be substituted;
X is a polar substituent;
and m is 0-2;

or a pharmaceutically acceptable salt thereof.

B2. The compound of embodiment B1, wherein Z¹ and Z² are each C.

B3. The compound of embodiment B1 or B2, wherein Z³-Z⁵ are each N.

B4. The compound of embodiment B1 or B2, wherein Z⁵ is CR⁵, and Z⁴ and Z³ are N or NR⁵.

B5. The compound of embodiment B1 or B2, wherein Z³ is CR⁵, and Z⁴ and Z⁵ are N or NR⁵.

B6. The compound of embodiment B1 or B2, wherein one of Z³-Z⁵ is O, and one of Z³-Z⁵ is N, and one of Z³-Z⁵ is CR⁵.

B7. The compound of any one of embodiments B1-B6, which is a compound of Formula IIa:

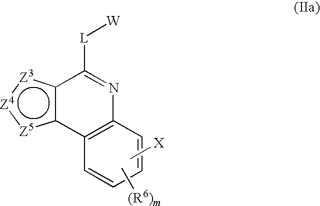

wherein one of Z³, Z⁴ and Z⁵ is either O or N, and the other two are selected from N and CR⁵, or a pharmaceutically acceptable salt thereof.

B8. The compound of embodiment B7, wherein Z³ is O, while Z⁴ and Z⁵ are each CR⁵.

B9. The compound of embodiment B7, wherein Z⁵ is O, while Z⁴ and Z³ are each CR⁵.

B10. The compound of embodiment B1, wherein one of Z¹ and Z² is N, the other of Z¹ and Z² is C.

B 11. The compound of embodiment B1 or B 10, wherein Z¹ is C and Z² is N.

B12. The compound of embodiment B1 or B10, wherein Z¹ is N and Z² is C.

B13. The compound of embodiment B10, B11 or B12, wherein at least one of Z³-Z⁵ is N.

B 14. The compound of embodiment B 10, B 11 or B 12, wherein each of Z³-Z⁵ is CR⁵.

B15. The compound of embodiment B1, having the structure of formula IIb or formula IIc:

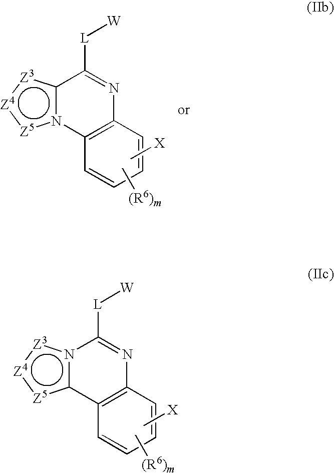

wherein Z³, Z⁴ and Z⁵ are independently selected from N and CR⁵, and the ring containing Z³-Z⁵ is aromatic;

L is a linker selected from a bond, NR³, O, S, CR³R⁴, CR³R⁴—NR³, CR³R⁴—O—, and CR³R⁴—S;
where each R³, R⁴, R⁵, and R⁶ is independently H, or an optionally substituted member selected from the group consisting of C1-C8 alkyl, C2-C8 heteroalkyl, C2-C8 alkenyl, C2-C8 heteroalkenyl, C2-C8 alkynyl, C2-C8 heteroalkynyl, C1-C8 acyl, C2-C8 heteroacyl, C6-C10 aryl, C5-C12 heteroaryl, C7-C12 arylalkyl, and C6-C12 heteroarylalkyl group,
   or halo, OR, NR₂, NROR, NRNR₂, SR, SOR, SO₂R, SO₂NR₂, NRSO₂R, NRCONR₂, NRCSNR₂, NRC(═NR)NR₂, NRCOOR, NRCOR, CN, COOR, CONR₂, OOCR, COR, or NO₂,
   wherein each R is independently H or C1-C8 alkyl, C2-C8 heteroalkyl, C2-C8 alkenyl, C2-C8 heteroalkenyl, C2-C8 alkynyl, C2-C8 heteroalkynyl, C1-C8 acyl, C2-C8 heteroacyl, C6-C10 aryl, C5-C10 heteroaryl, C7-C12 arylalkyl, or C6-C12 heteroarylalkyl,
   and wherein two R on the same atom or on adjacent atoms can be linked to form a 3-8 membered ring, optionally containing one or more N, O or S;
      and each R group, and each ring formed by linking two R groups together, is optionally substituted with one or more substituents selected from halo, ═O, ═N—CN, ═N—OR′, ═NR′, OR′, NR′₂, SR′, SO₂R′, SO₂NR′₂, NR′SO₂R′, NR′CONR′₂, NR′CSNR′₂, NR′C(═NR′)NR′₂, NR′COOR′, NR′COR′, CN, COOR′, CONR′₂, OOCR′, COR′, and NO₂,
      wherein each R′ is independently H, C1-C6 alkyl, C2-C6 heteroalkyl, C1-C6 acyl, C2-C6 heteroacyl, C6-C10 aryl, C5-C10 heteroaryl, C7-12 arylalkyl, or C6-12 heteroarylalkyl, each of which is optionally substituted with one or more groups selected from halo, C1-C4 alkyl, C1-C4 heteroalkyl, C1-C6 acyl, C1-C6 heteroacyl, hydroxy, amino, and ═O;
         and wherein two R′ on the same atom or on adjacent atoms can be linked to form a 3-7 membered ring optionally containing up to three heteroatoms selected from N, O and S;
   and R³ and R⁴, when on the same atom or on adjacent connected atoms, can optionally be linked together to form a 3-8 membered cycloalkyl or heterocycloalkyl, which is optionally substituted;
W is alkyl, heteroalkyl, aryl, heteroaryl, cycloalkyl, or heterocyclyl, each of which can be substituted;
X is a polar substituent;
and m is 0-2;

or a pharmaceutically acceptable salt thereof.

B16. The compound of embodiment B15, wherein at least one of Z³-Z⁵ is N.

B17. The compound of embodiment B15, wherein each of Z³-Z⁵ is CR⁵

B18. The compound of embodiment B15, having the structure of formula IIb wherein at least one of Z³-Z⁵ is N and the others are CR⁵.

B19. The compound of embodiment B15, having the structure of formula IIb wherein each of Z³-Z⁵ is CR⁵.

B20. The compound of embodiment B 15, having the structure of formula IIc wherein at least one of Z³-Z⁵ is N and the others are CR⁵.

B21. The compound of embodiment B15, having the structure of formula Jib wherein each of Z³-Z⁵ is CR⁵.

B22. The compound of any one of embodiments B1-B21, wherein L is NH or NMe.

B23. The compound of any one of embodiments B1-B22, wherein W is selected from optionally substituted alkyl, optionally substituted aryl, optionally substituted heterocyclyl, and optionally substituted cycloalkyl.

B24. The compound of any one of embodiments B1-B23, wherein L is NH or NMe, and W is optionally substituted phenyl, optionally substituted heterocyclyl, or C1-C4 alkyl substituted with at least one member selected from the group consisting of optionally substituted phenyl, optionally substituted heteroalkyl, optionally substituted heteroaryl, halo, and —NR″₂,

where each R″ is independently H or optionally substituted C1-C6 alkyl;
or two R″ taken together with the N to which they are attached can be linked together to form an optionally substituted 3-8 membered ring, which can contain another heteroatom selected from N, O and S as a ring member, and can be saturated, unsaturated or aromatic.

B25. The compound of any one of embodiments B1-B24, wherein W is optionally substituted phenyl, optionally substituted heterocyclyl, or C1-C4 alkyl substituted with at least one member selected from the group consisting of optionally substituted phenyl, optionally substituted heteroalkyl, optionally substituted heteroaryl, halo, and —NR″₂,

where each R″ is independently H or optionally substituted C1-C6 alkyl;
or two R″ taken together with the N to which they are attached can be linked together to form an optionally substituted 3-8 membered ring, which can contain another heteroatom selected from N, O and S as a ring member, and can be saturated, unsaturated or aromatic.

B26. The compound of embodiment B25, wherein W comprises at least one group of the formula —(CH₂)ₚ—NR^{x}₂,

where p is 1-4,

R^{x} is independently at each occurrence H or optionally substituted alkyl;
or two R^{x} taken together with the N to which they are attached can be linked together to form an optionally substituted 3-8 membered ring, which can contain another heteroatom selected from N, O and S as a ring member, and can be saturated, unsaturated or aromatic.

B27. The compound of any one of embodiments B1-B26, wherein L is NH or NMe, and

W is optionally substituted phenyl, optionally substituted heterocyclyl, or C1-C4 alkyl substituted with at least one member selected from the group consisting of optionally substituted phenyl, optionally substituted heteroalkyl, optionally substituted heteroaryl, halo, and —NR″₂,

where each R″ is independently H or optionally substituted C1-C6 alkyl;

or two R″ taken together with the N to which they are attached can be linked together to form an optionally substituted 3-8 membered ring, which can contain another heteroatom selected from N, O and S as a ring member, and can be saturated, unsaturated or aromatic.

B28. The compound of any one of embodiments B1-B27, wherein X is selected from the group consisting of COOR9, C(O)NR9-OR9, triazole, tetrazole, CN, imidazole, carboxylate, a carboxylate bioisostere,

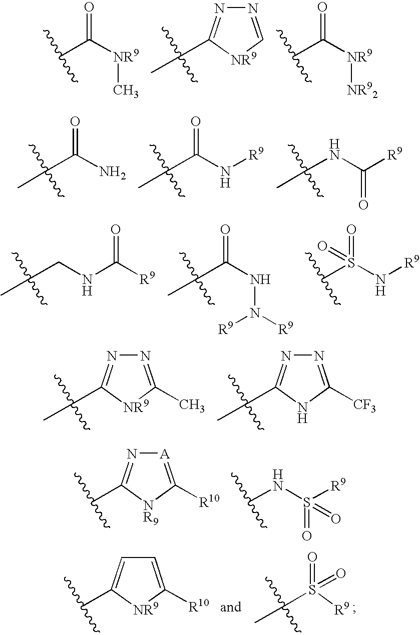

wherein each R⁹ is independently H or an optionally substituted member selected from the group consisting of alkyl, cycloalkyl, heterocyclyl, aryl, heteroaryl, arylalkyl, cycloalkylalkyl, heterocycloalkylalkyl, and heteroarylalkyl,
   and two R⁹ on the same or adjacent atoms can optionally be linked together to form an optionally substituted ring that can also contain an additional heteroatom selected from N, O and S as a ring member;
R¹⁰ is halo, CF₃, CN, SR, OR, NR₂, or R, where each R is independently H or optionally substituted C1-C6 alkyl, and two R on the same or adjacent atoms can optionally be linked together to form an optionally substituted ring that can also contain an additional heteroatom selected from N, O and S as a ring member;
and A is N or CR¹⁰.

B29. The compound of any one of embodiments B1-B28, wherein the polar substituent X is located at position 3 on the phenyl ring.

B30. The compound of any one of embodiments B1-B28, wherein the polar substituent X is located at position 4 on the phenyl ring.

B31. The compound of any one of embodiments B1-B30, wherein -L-W is selected from:

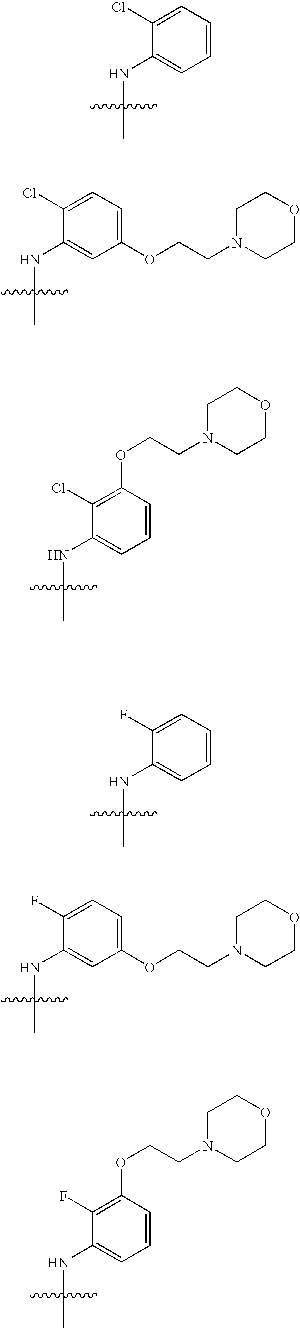

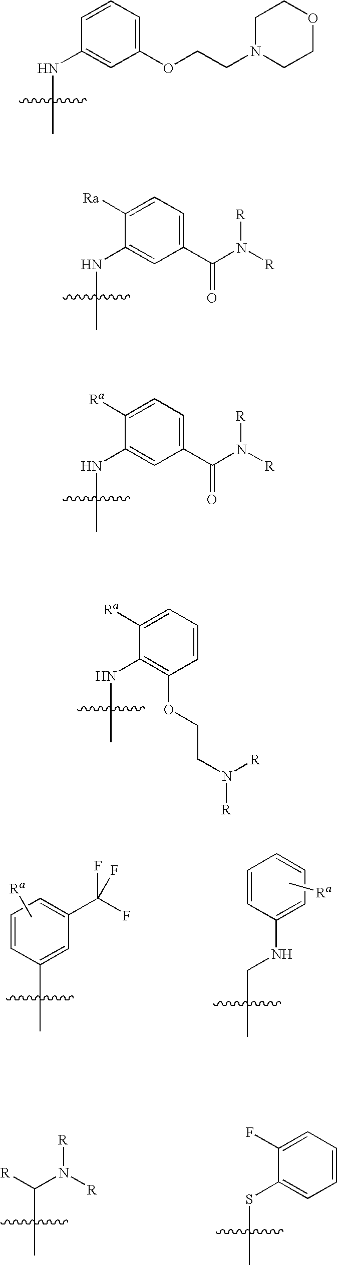

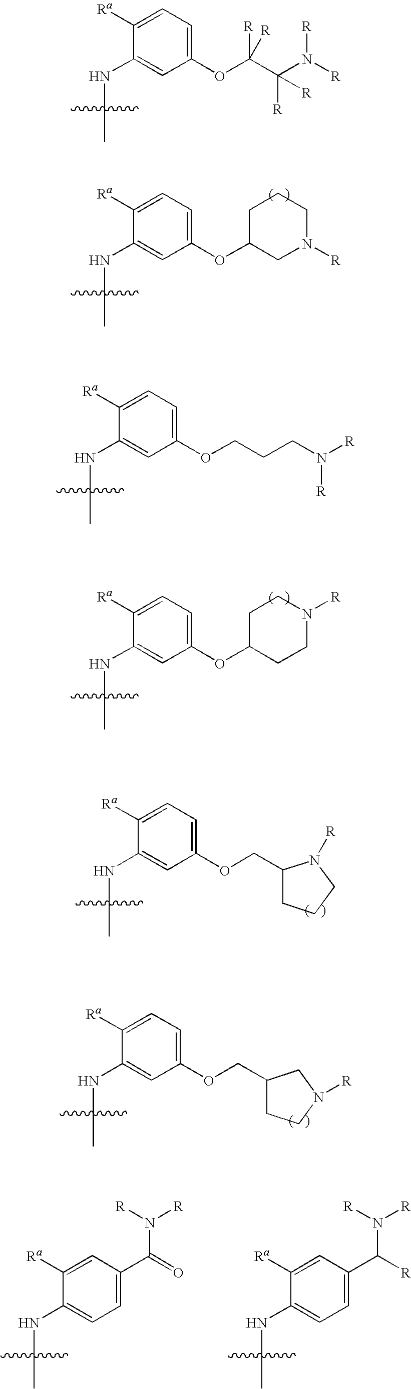

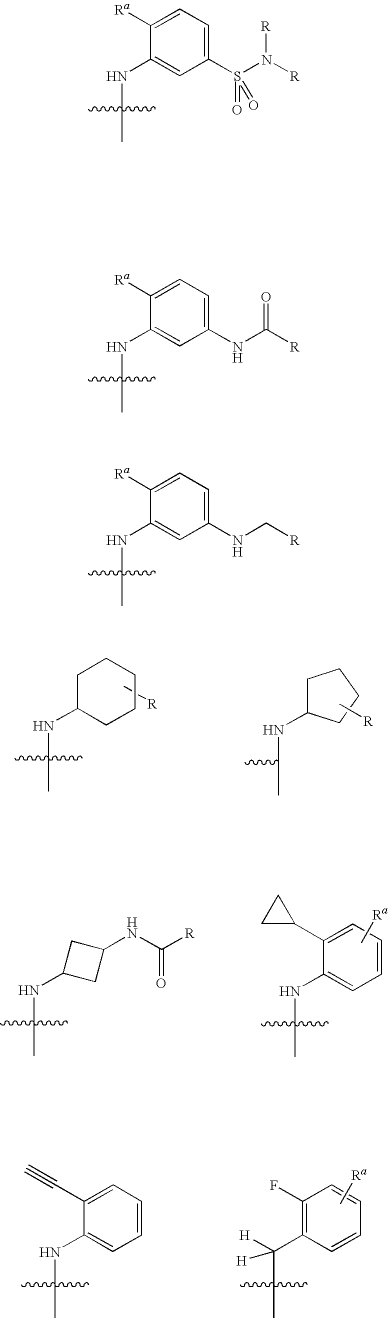

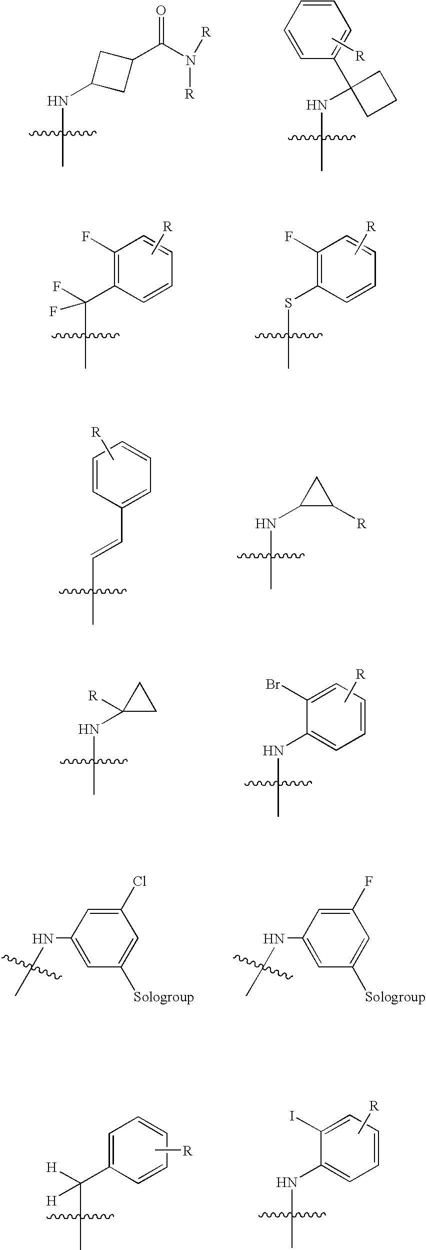

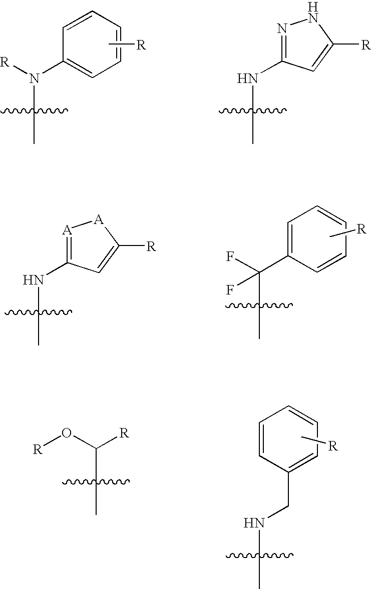

wherein each R¹ is H, Cl or F;
each R is independently selected from halo, C1-C4 alkyl, C1-C4 alkoxy, and C1-C4 haloalkyl,
   and two R groups on the same or adjacent connected atoms can optionally be linked together to form a 3-8 membered ring;
each A is N or CR;

and each Solgroup is a solubility-enhancing group.

C1. A compound, which is any of the species disclosed herein; or a pharmaceutically acceptable salt thereof.

C2. A pharmaceutical composition comprising a compound of formula I, Ia, II, IIa, IIb or IIc, and a pharmaceutically acceptable excipient.

C3. A pharmaceutical composition comprising a compound of any one of embodiments A1-A25 and a pharmaceutically acceptable excipient.

C4. A pharmaceutical composition comprising a compound of any one of embodiments B1-B31 and a pharmaceutically acceptable excipient.

C5. A pharmaceutical composition comprising a compound of any one of embodiments A1-A25 or B1-B31 and at least two pharmaceutically acceptable excipients.

D1. A method for inhibiting cell proliferation, which comprises contacting cells with a compound having a structure of Formula I, Ia, II, IIa, IIb or IIc in an amount effective to inhibit proliferation of the cells.

D2. The method of embodiment D1, wherein the compound comprises any one of embodiments A1-A25 or B1-B31.

D3. The method of embodiment D1 or D2, wherein the cells are in a cancer cell line.

D4. The method of embodiment D3, wherein the cancer cell line is a breast cancer, prostate cancer, pancreatic cancer, lung cancer, hemopoietic cancer, colorectal cancer, skin cancer, ovary cancer cell line.

D5. The method of embodiment D1 or D2, wherein the cells are in a tumor in a subject.

D6. The method of embodiment D1, wherein contacting cells with a compound having a structure of Formula I, Ia, II, IIa, IIb or IIc induces cell apoptosis.

D7. The method of embodiment D6, wherein the compound comprises any one of embodiments A1-A25 or B1-B31.

D8. The method of embodiment D1 or D2, wherein the cells are from an eye of a subject having macular degeneration.

D9. The method of embodiment D1 or D2, wherein the cells are in a subject having macular degeneration.

D10. A method for treating a condition related to aberrant cell proliferation, which comprises administering a compound having a structure of Formula I, Ia, II, IIa, IIb or IIc to a subject in need thereof in an amount effective to treat the cell proliferative condition.

D11. The method of embodiment D10, wherein the compound comprises any one of embodiments A1-A25 or B1-B31.

D12. The method of embodiment D10 or D11, wherein the cell proliferative condition is a tumor-associated cancer.

D13. The method of embodiment D12, wherein the cancer is of the colorectum, breast, lung, liver, pancreas, lymph node, colon, prostate, brain, head and neck, skin, liver, kidney, blood and heart.

D14. The method of embodiment D10 or D11, wherein the cell proliferative condition is a non-tumor cancer.

D15. The method of embodiment D14, wherein the non-tumor cancer is a hematopoietic cancer.

D16. The method of embodiment D10 or D11, wherein the cell proliferative condition is macular degeneration.

D17. A method for treating pain or inflammation in a subject, which comprises administering a compound of Formula I, Ia, II, IIa, IIb or IIc to a subject in need thereof in an amount effective to treat the pain or the inflammation.

D18. The method of embodiment D17, wherein the compound comprises any one of embodiments A1-A25 or B1-B31.

D19. A method for inhibiting angiogenesis in a subject, which comprises administering a compound of Formula I, Ia, II, IIa, IIb or IIc to a subject in need thereof in an amount effective to inhibit the angiogenesis.

D20. The method of embodiment D19, wherein the compound comprises any one of embodiments A1-A25 or B1-B31.

1. A compound of Formula II: 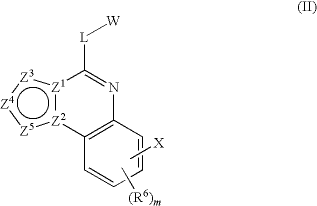
wherein Z¹ and Z² are each C, or one of Z¹ and Z² is N, the other of Z¹ and Z² is C;
Z³, Z⁴ and Z⁵ are independently selected from N, NR⁵, CR⁵ and O, provided not more than one of Z³-Z⁵ is O, and the ring containing Z³-Z⁵ is aromatic;
L is a linker selected from a bond, NR³, O, S, CR³R⁴, CR³R⁴—NR³, CR³R⁴—O—, and CR³R⁴—S;
where each R³, R⁴, R⁵, and R⁶ is independently H, or an optionally substituted member selected from the group consisting of C1-C8 alkyl, C2-C8 heteroalkyl, C2-C8 alkenyl, C2-C8 heteroalkenyl, C2-C8 alkynyl, C2-C8 heteroalkynyl, C1-C8 acyl, C2-C8 heteroacyl, C6-C10 aryl, C5-C12 heteroaryl, C7-C12 arylalkyl, and C6-C12 heteroarylalkyl group,
or halo, OR, NR₂, NROR, NRNR₂, SR, SOR, SO₂R, SO₂NR₂, NRSO₂R, NRCONR₂, NRCSNR₂, NRC(═NR)NR₂, NRCOOR, NRCOR, CN, COOR, CONR₂, OOCR, COR, or NO₂,
wherein each R is independently H or C1-C8 alkyl, C2-C8 heteroalkyl, C2-C8 alkenyl, C2-C8 heteroalkenyl, C2-C8 alkynyl, C2-C8 heteroalkynyl, C1-C8 acyl, C2-C8 heteroacyl, C6-C10 aryl, C5-C10 heteroaryl, C7-C12 arylalkyl, or C6-C12 heteroarylalkyl,
and wherein two R on the same atom or on adjacent atoms can be linked to form a 3-8 membered ring, optionally containing one or more N, O or S;
and each R group, and each ring formed by linking two R groups together, is optionally substituted with one or more substituents selected from halo, ═O, ═N—CN, ═N—OR′, ═NR′, OR′, NR′₂, SR′, SO₂R′, SO₂NR′₂, NR′SO₂R′, NR′CONR′₂, NR′CSNR′₂, NR′C(═NR′)NR′₂, NR′COOR′, NR′COR′, CN, COOR′, CONR′₂, OOCR′, COR′, and NO₂,
wherein each R′ is independently H, C1-C6 alkyl, C2-C6 heteroalkyl, C1-C6 acyl, C2-C6 heteroacyl, C6-C10 aryl, C5-C10 heteroaryl, C7-12 arylalkyl, or C6-12 heteroarylalkyl, each of which is optionally substituted with one or more groups selected from halo, C1-C4 alkyl, C1-C4 heteroalkyl, C1-C6 acyl, C1-C6 heteroacyl, hydroxy, amino, and ═O;
and wherein two R′ on the same atom or on adjacent atoms can be linked to form a 3-7 membered ring optionally containing up to three heteroatoms selected from N, O and S;
and R³ and R⁴, when on the same atom or on adjacent connected atoms, can optionally be linked together to form a 3-8 membered cycloalkyl or heterocycloalkyl, which is optionally substituted;
W is alkyl, heteroalkyl, aryl, heteroaryl, cycloalkyl, or heterocyclyl, each of which can be substituted;
X is a polar substituent;
and m is 0-2;
or a pharmaceutically acceptable salt thereof.

2. The compound of claim 1, wherein one of Z¹ and Z² is N and the other of Z¹ and Z² is C.

3. The compound of claim 1, wherein Z¹ and Z² are each C.

4. The compound of claim 1, wherein L is NH or NMe.

5. The compound of claim 1, wherein W is selected from optionally substituted alkyl, optionally substituted aryl, optionally substituted heterocyclyl, and optionally substituted cycloalkyl.

6. The compound of claim 1, wherein W is optionally substituted phenyl, optionally substituted heterocyclyl, or C1-C4 alkyl substituted with at least one member selected from the group consisting of optionally substituted phenyl, optionally substituted heteroalkyl, optionally substituted heteroaryl, halo, and —NR″₂,
where each R″ is independently H or optionally substituted C1-C6 alkyl;
or two R″ taken together with the N to which they are attached can be linked together to form an optionally substituted 3-8 membered ring, which can contain another heteroatom selected from N, O and S as a ring member, and can be saturated, unsaturated or aromatic.

7. The compound of claim 1, wherein W comprises at least one group of the formula —(CH₂)ₚ—NR^{x}₂,
where p is 1-4,
R^{x} is independently at each occurrence H or optionally substituted alkyl;
or two R^{x} taken together with the N to which they are attached can be linked together to form an optionally substituted 3-8 membered ring, which can contain another heteroatom selected from N, O and S as a ring member, and can be saturated, unsaturated or aromatic.

8. The compound of claim 1, wherein X is selected from the group consisting of COOR⁹, C(O)NR⁹—OR⁹, triazole, tetrazole, CN, imidazole, carboxylate, a carboxylate bioisostere, 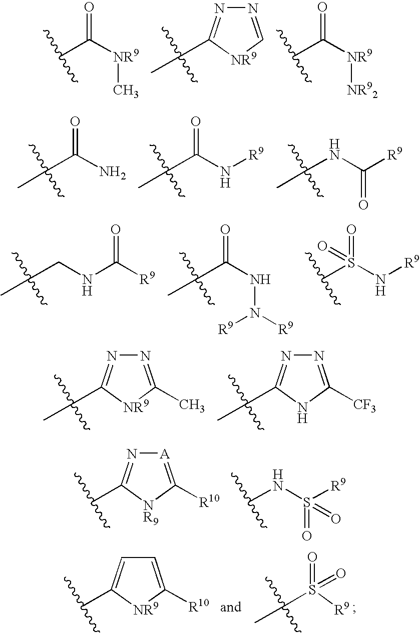
wherein each R⁹ is independently H or an optionally substituted member selected from the group consisting of alkyl, cycloalkyl, heterocyclyl, aryl, heteroaryl, arylalkyl, cycloalkylalkyl, heterocycloalkylalkyl, and heteroarylalkyl,
and two R⁹ on the same or adjacent atoms can optionally be linked together to form an optionally substituted ring that can also contain an additional heteroatom selected from N, O and S as a ring member;
R¹⁰ is halo, CF₃, CN, SR, OR, NR₂, or R, where each R is independently H or optionally substituted C1-C6 alkyl, and two R on the same or adjacent atoms can optionally be linked together to form an optionally substituted ring that can also contain an additional heteroatom selected from N, O and S as a ring member;
and A is N or CR¹⁰.

9. The compound of claim 1, wherein the polar substituent X is located at position 3 or position 4 on the phenyl ring.

10. The compound of claim 1, wherein -L-W is selected from: 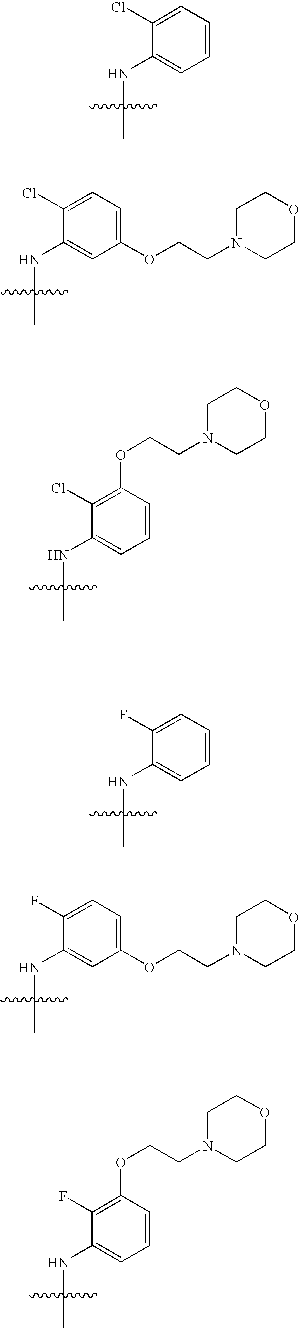 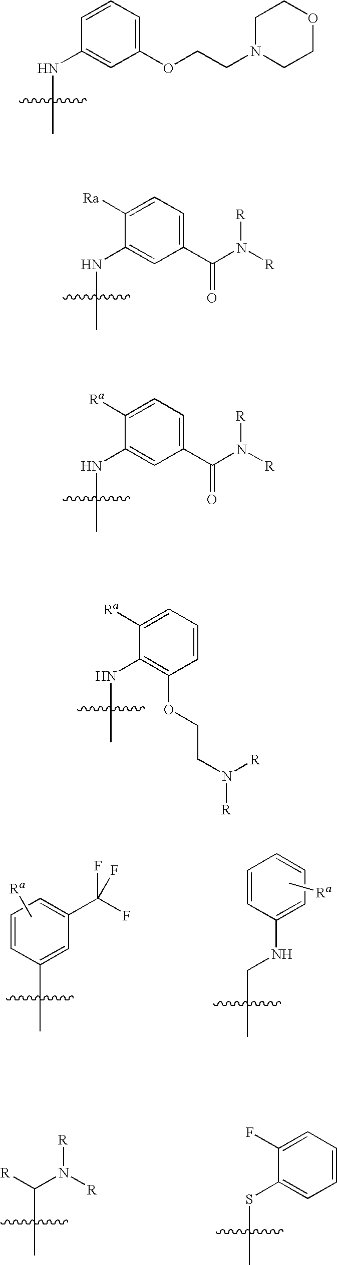 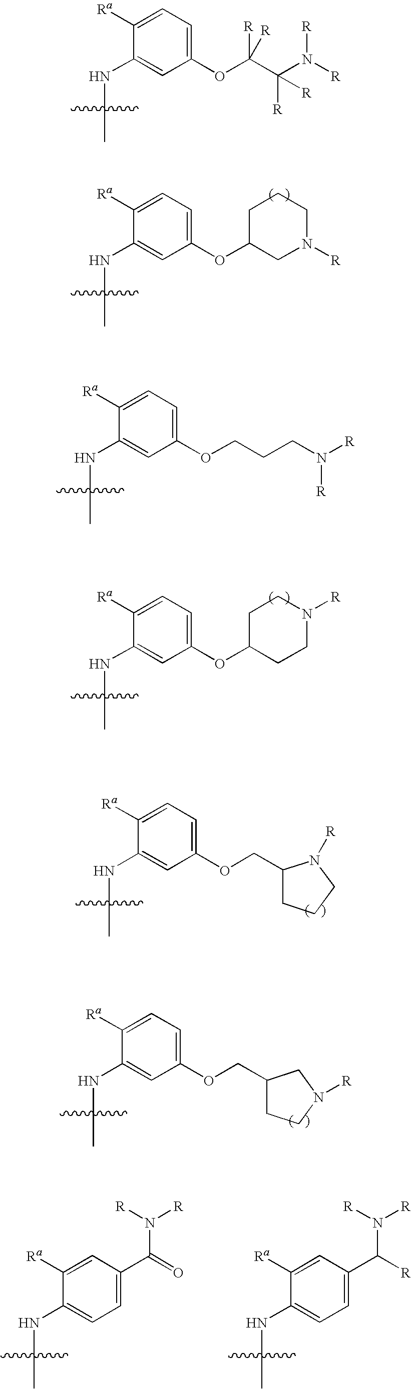 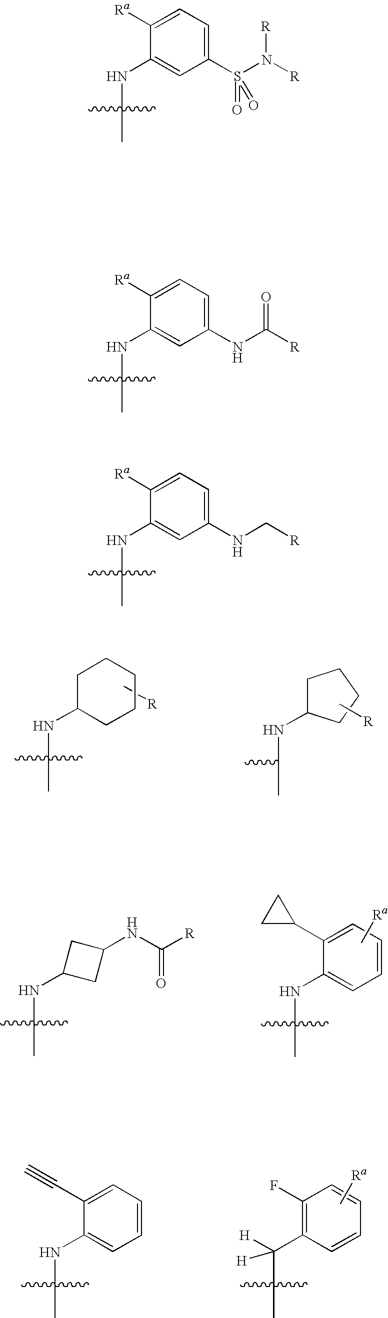 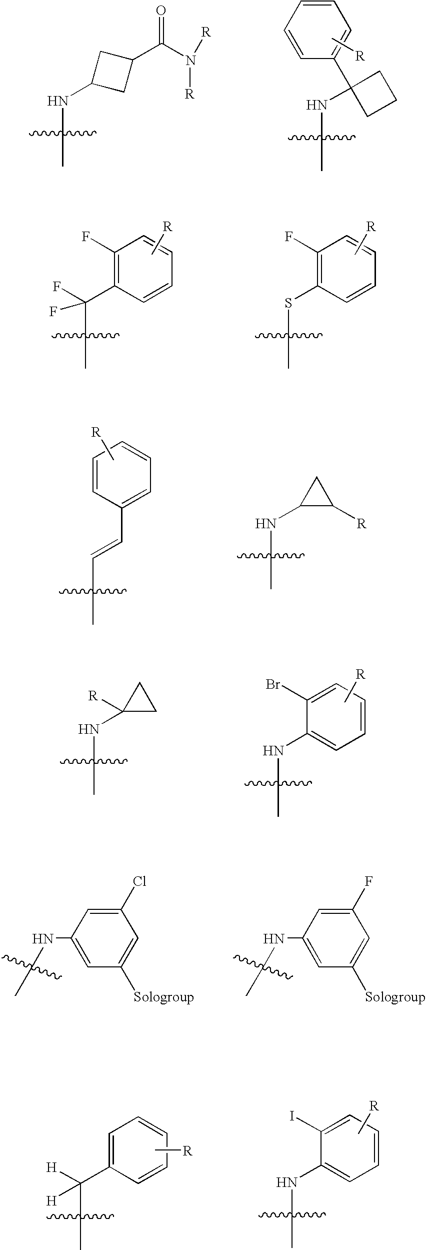 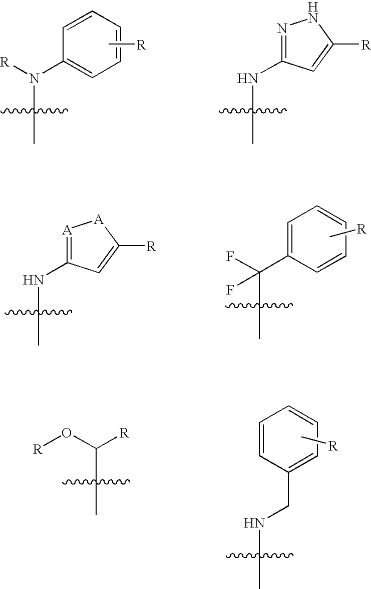
wherein each R^{a} is H, Cl or F;
each R is independently selected from halo, C1-C4 alkyl, C1-C4 alkoxy, and C1-C4 haloalkyl,
and two R groups on the same or adjacent connected atoms can optionally be linked together to form a 3-8 membered ring;
each A is N or CR;
and each Solgroup is a solubility-enhancing group.

11. The compound of claim 1, having the structure of formula IIb or formula IIc: 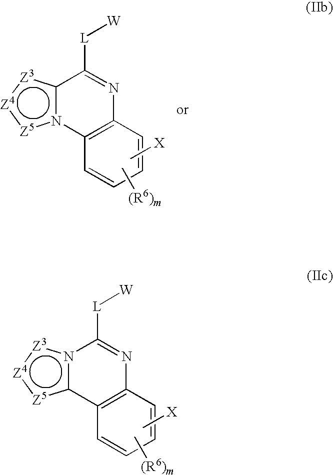
wherein Z³, Z⁴ and Z⁵ are independently selected from N and CR⁵, and the ring containing Z³-Z⁵ is aromatic;
L is a linker selected from a bond, NR³, O, S, CR³R⁴, CR³R⁴—NR³, CR³R⁴—O—, and CR³R⁴—S;
where each R³, R⁴, R⁵, and R⁶ is independently H, or an optionally substituted member selected from the group consisting of C1-C8 alkyl, C2-C8 heteroalkyl, C2-C8 alkenyl, C2-C8 heteroalkenyl, C2-C8 alkynyl, C2-C8 heteroalkynyl, C1-C8 acyl, C2-C8 heteroacyl, C6-C10 aryl, C5-C12 heteroaryl, C7-C12 arylalkyl, and C6-C12 heteroarylalkyl group,
or halo, OR, NR₂, NROR, NRNR₂, SR, SOR, SO₂R, SO₂NR₂, NRSO₂R, NRCONR₂, NRCSNR₂, NRC(═NR)NR₂, NRCOOR, NRCOR, CN, COOR, CONR₂, OOCR, COR, or NO₂,
wherein each R is independently H or C1-C8 alkyl, C2-C8 heteroalkyl, C2-C8 alkenyl, C2-C8 heteroalkenyl, C2-C8 alkynyl, C2-C8 heteroalkynyl, C1-C8 acyl, C2-C8 heteroacyl, C6-C10 aryl, C5-C10 heteroaryl, C7-C12 arylalkyl, or C6-C12 heteroarylalkyl,
and wherein two R on the same atom or on adjacent atoms can be linked to form a 3-8 membered ring, optionally containing one or more N, O or S;
and each R group, and each ring formed by linking two R groups together, is optionally substituted with one or more substituents selected from halo, ═O, ═N—CN, ═N—OR′, ═NR′, OR′, NR′₂, SR′, SO₂R′, SO₂NR′₂, NR′SO₂R′, NR′CONR′₂, NR′CSNR′₂, NR′C(═NR′)NR′₂, NR′COOR′, NR′COR′, CN, COOR′, CONR′₂, OOCR′, COR′, and NO₂,
wherein each R′ is independently H, C1-C6 alkyl, C2-C6 heteroalkyl, C1-C6 acyl, C2-C6 heteroacyl, C6-C10 aryl, C5-C10 heteroaryl, C7-12 arylalkyl, or C6-12 heteroarylalkyl, each of which is optionally substituted with one or more groups selected from halo, C1-C4 alkyl, C1-C4 heteroalkyl, C1-C6 acyl, C1-C6 heteroacyl, hydroxy, amino, and ═O;
and wherein two R′ on the same atom or on adjacent atoms can be linked to form a 3-7 membered ring optionally containing up to three heteroatoms selected from N, O and S;
and R³ and R⁴, when on the same atom or on adjacent connected atoms, can optionally be linked together to form a 3-8 membered cycloalkyl or heterocycloalkyl, which is optionally substituted;
W is alkyl, heteroalkyl, aryl, heteroaryl, cycloalkyl, or heterocyclyl, each of which can be substituted;
X is a polar substituent;
and m is 0-2;
or a pharmaceutically acceptable salt thereof.

12. The compound of claim 11, having the structure of formula IIb wherein at least one of Z³-Z⁵ is N and the others are CR⁵.

13. The compound of claim 11, having the structure of formula IIc wherein at least one of Z³-Z⁵ is N and the others are CR⁵.

14. The compound of claim 1, which is a compound of Formula IIa: 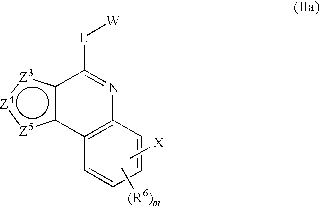
wherein one of Z³, Z⁴ and Z⁵ is either O or N, and the other two are selected from N and CR⁵, or a pharmaceutically acceptable salt thereof.

15. A compound of claim 1, which is any of the species disclosed herein; or a pharmaceutically acceptable salt thereof.

16. A pharmaceutical composition comprising a compound of claim 1 and a pharmaceutically acceptable excipient.

17. A method to treat cancer, a vascular disorder, pain, inflammation, or a pathogenic infection, comprising administering to a subject in need of such treatment, an effective amount of the compound of claim 1.

18. A method to treat a disorder associated with excessive CK2 or Pim kinase activity by administering to a subject in need of such treatment an effective amount of a compound according to claim 1.

19. The method of claim 18, wherein the disorder is selected from cancer, a vascular disorder, a pathogenic infection, and an immunological disorder.

20. A compound having a structure of Formula I: 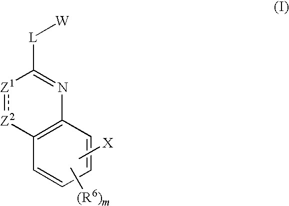
wherein:
Z¹ and Z² are independently selected from N, NR¹, C═V, and CR², provided Z¹ and Z² are not both NR¹;
where R¹ and R² are independently selected from H, optionally substituted amino, optionally substituted alkoxy, optionally substituted alkyl, optionally substituted alkenyl, optionally substituted aryl, optionally substituted heteroaryl, optionally substituted heteroalkyl, optionally substituted heteroalkenyl, optionally substituted cycloalkyl, and optionally substituted heterocycloalkyl;
C═V is C═CR¹R⁵ or C═NR¹;
and two R¹ groups, or R¹ and R² if both are present, can be taken together to form a 5-6 membered optionally substituted heterocyclic ring;
L is a linker selected from a bond, NR³, O, S, CR³R⁴, CR³R⁴—NR³, CR³R⁴—O—, and CR³R⁴—S;
where each R³, R⁴, R⁵, and R⁶ is independently H, or an optionally substituted member selected from the group consisting of C1-C8 alkyl, C2-C8 heteroalkyl, C2-C8 alkenyl, C2-C8 heteroalkenyl, C2-C8 alkynyl, C2-C8 heteroalkynyl, C1-C8 acyl, C2-C8 heteroacyl, C6-C10 aryl, C5-C12 heteroaryl, C7-C12 arylalkyl, and C6-C12 heteroarylalkyl group,
or halo, OR, NR₂, NROR, NRNR₂, SR, SOR, SO₂R, SO₂NR₂, NRSO₂R, NRCONR₂, NRCSNR₂, NRC(═NR)NR₂, NRCOOR, NRCOR, CN, COOR, CONR₂, OOCR, COR, or NO₂,
wherein each R is independently H or C1-C8 alkyl, C2-C8 heteroalkyl, C2-C8 alkenyl, C2-C8 heteroalkenyl, C2-C8 alkynyl, C2-C8 heteroalkynyl, C1-C8 acyl, C2-C8 heteroacyl, C6-C10 aryl, C5-C10 heteroaryl, C7-C12 arylalkyl, or C6-C12 heteroarylalkyl,
and wherein two R on the same atom or on adjacent atoms can be linked to form a 3-8 membered ring, optionally containing one or more N, O or S;
and each R group, and each ring formed by linking two R groups together, is optionally substituted with one or more substituents selected from halo, ═O, ═N—CN, ═N—OR′, ═NR′, OR′, NR′₂, SR′, SO₂R′, SO₂NR′₂, NR′SO₂R′, NR′CONR′₂, NR′CSNR′₂, NR′C(═NR′)NR′₂, NR′COOR′, NR′COR′, CN, COOR′, CONR′₂, OOCR′, COR′, and NO₂,
wherein each R′ is independently H, C1-C6 alkyl, C2-C6 heteroalkyl, C1-C6 acyl, C2-C6 heteroacyl, C6-C10 aryl, C5-C10 heteroaryl, C7-12 arylalkyl, or C6-12 heteroarylalkyl, each of which is optionally substituted with one or more groups selected from halo, C1-C4 alkyl, C1-C4 heteroalkyl, C1-C6 acyl, C1-C6 heteroacyl, hydroxy, amino, and ═O;
and wherein two R′ on the same atom or on adjacent atoms can be linked to form a 3-7 membered ring optionally containing up to three heteroatoms selected from N, O and S;
and R³ and R⁴, when on the same atom or on adjacent connected atoms, can optionally be linked together to form a 3-8 membered cycloalkyl or heterocycloalkyl, which is optionally substituted;
W is alkyl, heteroalkyl, aryl, heteroaryl, cycloalkyl, or heterocyclyl, each of which can be optionally substituted;
X is a polar substituent;
and m is 0-2;
or a pharmaceutically acceptable salt thereof.
